SEQUENCE LISTING (110 sequences)

SEQ ID NO: 1 - DNA, 20 bp, synthetic construct
  source 1..20: mol_type = other DNA
ccagctgaaacaaaggaagg

SEQ ID NO: 2 - DNA, 21 bp, synthetic construct
  source 1..21: mol_type = other DNA
cagggaaacactgtctttgga

SEQ ID NO: 3 - DNA, 20 bp, synthetic construct
  source 1..20: mol_type = other DNA
tgggtgtcacttttcattgc

SEQ ID NO: 4 - DNA, 20 bp, synthetic construct
  source 1..20: mol_type = other DNA
atgcgcttgccatctttgta

SEQ ID NO: 5 - DNA, 22 bp, synthetic construct
  source 1..22: mol_type = other DNA
agaagatgaaggaacttacacg

SEQ ID NO: 6 - DNA, 23 bp, synthetic construct
  source 1..23: mol_type = other DNA
ggatttgtggtctatgtcttcag

SEQ ID NO: 7 - DNA, 352 bp, synthetic construct
  source 1..352: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnngttttagagctagaaatagcaagttaaaataaggctagtccgttatcaacttgaaaaagtggcaccgagtcggtgctttttt

SEQ ID NO: 8 - DNA, 5733 bp, synthetic construct
  source 1..5733: mol_type = other DNA
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgacatggacttctccgcactgctgggtagcggatcgggatctcgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttga

SEQ ID NO: 9 - DNA, 20 bp, synthetic construct
  source 1..20: mol_type = other DNA
agttttgtttttaggattag

SEQ ID NO: 10 - DNA, 20 bp, synthetic construct
  source 1..20: mol_type = other DNA
gggtctggcctgcccttata

SEQ ID NO: 11 - DNA, 20 bp, synthetic construct
  source 1..20: mol_type = other DNA
tataagggcaggccagaccc

SEQ ID NO: 12 - DNA, 20 bp, synthetic construct
  source 1..20: mol_type = other DNA
gcccttatattcaccagatc

SEQ ID NO: 13 - DNA, 20 bp, synthetic construct
  source 1..20: mol_type = other DNA
ttatattcaccagatctgct

SEQ ID NO: 14 - DNA, 20 bp, synthetic construct
  source 1..20: mol_type = other DNA
cagcagatctggtgaatata

SEQ ID NO: 15 - DNA, 20 bp, synthetic construct
  source 1..20: mol_type = other DNA
gctgacagccccacatcctt

SEQ ID NO: 16 - DNA, 20 bp, synthetic construct
  source 1..20: mol_type = other DNA
acagccccacatccttaggc

SEQ ID NO: 17 - DNA, 20 bp, synthetic construct
  source 1..20: mol_type = other DNA
agggccctgcctaaggatgt

SEQ ID NO: 18 - DNA, 20 bp, synthetic construct
  source 1..20: mol_type = other DNA
ctaagacagccaatcaccca

SEQ ID NO: 19 - DNA, 20 bp, synthetic construct
  source 1..20: mol_type = other DNA
gggtgattggctgtcttagg

SEQ ID NO: 20 - DNA, 20 bp, synthetic construct
  source 1..20: mol_type = other DNA
gctgtcttaggaaagcatga

SEQ ID NO: 21 - DNA, 20 bp, synthetic construct
  source 1..20: mol_type = other DNA
ctgtcttaggaaagcatgat

SEQ ID NO: 22 - DNA, 20 bp, synthetic construct
  source 1..20: mol_type = other DNA
ttaggaaagcatgatgggag

SEQ ID NO: 23 - DNA, 20 bp, synthetic construct
  source 1..20: mol_type = other DNA
atgatgggagaggacctatt

SEQ ID NO: 24 - DNA, 20 bp, synthetic construct
  source 1..20: mol_type = other DNA
atttggtagtgtcacatacc

SEQ ID NO: 25 - DNA, 20 bp, synthetic construct
  source 1..20: mol_type = other DNA
ggtatgtgacactaccaaat

SEQ ID NO: 26 - DNA, 20 bp, synthetic construct
  source 1..20: mol_type = other DNA
ctgtcaatcagaagaacagg

SEQ ID NO: 27 - DNA, 20 bp, synthetic construct
  source 1..20: mol_type = other DNA
gttcttctgattgacagtgc

SEQ ID NO: 28 - DNA, 20 bp, synthetic construct
  source 1..20: mol_type = other DNA
gcactgtcaatcagaagaac

SEQ ID NO: 29 - DNA, 20 bp, synthetic construct
  source 1..20: mol_type = other DNA
tgctaaatttagtacctcaa

SEQ ID NO: 30 - DNA, 20 bp, synthetic construct
  source 1..20: mol_type = other DNA
ccttggtgaagtctcctttg

SEQ ID NO: 31 - DNA, 20 bp, synthetic construct
  source 1..20: mol_type = other DNA
taaatacagttctatttcct

SEQ ID NO: 32 - DNA, 20 bp, synthetic construct
  source 1..20: mol_type = other DNA
aagtggattctggagaatgt

SEQ ID NO: 33 - DNA, 20 bp, synthetic construct
  source 1..20: mol_type = other DNA
ttgatacaaggttgacattt

SEQ ID NO: 34 - DNA, 20 bp, synthetic construct
  source 1..20: mol_type = other DNA
tgctgagacacaacttcccc

SEQ ID NO: 35 - DNA, 20 bp, synthetic construct
  source 1..20: mol_type = other DNA
accaggcacctaatttctcc

SEQ ID NO: 36 - DNA, 20 bp, synthetic construct
  source 1..20: mol_type = other DNA
acatatgatgtataagtttg

SEQ ID NO: 37 - DNA, 20 bp, synthetic construct
  source 1..20: mol_type = other DNA
gctgtgacatttatgaagct

SEQ ID NO: 38 - DNA, 20 bp, synthetic construct
  source 1..20: mol_type = other DNA
caagtagttgagataacttc

SEQ ID NO: 39 - DNA, 20 bp, synthetic construct
  source 1..20: mol_type = other DNA
tgcgatatctggaatgcaag

SEQ ID NO: 40 - DNA, 20 bp, synthetic construct
  source 1..20: mol_type = other DNA
ggaaggtgcaaagtgtttga

SEQ ID NO: 41 - DNA, 20 bp, synthetic construct
  source 1..20: mol_type = other DNA
cattttagattcaagaaaga

SEQ ID NO: 42 - DNA, 20 bp, Homo sapiens
  source 1..20: mol_type = genomic DNA
gaccatctccgacatgaatc

SEQ ID NO: 43 - DNA, 20 bp, Homo sapiens
  source 1..20: mol_type = genomic DNA
gttaaaatccgaaaatgcag

SEQ ID NO: 44 - DNA, 20 bp, Homo sapiens
  source 1..20: mol_type = genomic DNA
cttgtgggcagtcatacaaa

SEQ ID NO: 45 - DNA, 20 bp, Homo sapiens
  source 1..20: mol_type = genomic DNA
caaaagagccccaaacctga

SEQ ID NO: 46 - DNA, 19 bp, Homo sapiens
  source 1..19: mol_type = genomic DNA
caaaccaaatggacctgtc

SEQ ID NO: 47 - DNA, 20 bp, Homo sapiens
  source 1..20: mol_type = genomic DNA
cagatgaggaaatttatctc

SEQ ID NO: 48 - DNA, 20 bp, Homo sapiens
  source 1..20: mol_type = genomic DNA
tacactgatgttaatgagct

SEQ ID NO: 49 - DNA, 20 bp, Homo sapiens
  source 1..20: mol_type = genomic DNA
acaagatgacgccatgtact

SEQ ID NO: 50 - DNA, 20 bp, Homo sapiens
  source 1..20: mol_type = genomic DNA
atatttattaaacatatagt

SEQ ID NO: 51 - DNA, 20 bp, Homo sapiens
  source 1..20: mol_type = genomic DNA
ccataaaggtgactttttaa

SEQ ID NO: 52 - DNA, 20 bp, Homo sapiens
  source 1..20: mol_type = genomic DNA
aagcatttgtaaaggcgact

SEQ ID NO: 53 - DNA, 20 bp, Homo sapiens
  source 1..20: mol_type = genomic DNA
ttattttctgaattgtactg

SEQ ID NO: 54 - DNA, 20 bp, Homo sapiens
  source 1..20: mol_type = genomic DNA
ccccccaaacacacagaatt

SEQ ID NO: 55 - DNA, 20 bp, Homo sapiens
  source 1..20: mol_type = genomic DNA
tccataacacggcagttgat

SEQ ID NO: 56 - DNA, 20 bp, Homo sapiens
  source 1..20: mol_type = genomic DNA
gtgagctacattcctgcttc

SEQ ID NO: 57 - DNA, 20 bp, Homo sapiens
  source 1..20: mol_type = genomic DNA
taattttacaaatcggattc

SEQ ID NO: 58 - DNA, 20 bp, Homo sapiens
  source 1..20: mol_type = genomic DNA
catcaactagctatattccc

SEQ ID NO: 59 - DNA, 20 bp, Homo sapiens
  source 1..20: mol_type = genomic DNA
gtttgggtataccaagtttt

SEQ ID NO: 60 - DNA, 20 bp, Homo sapiens
  source 1..20: mol_type = genomic DNA
acaggtgctggagaggatgt

SEQ ID NO: 61 - DNA, 18 bp, Homo sapiens
  source 1..18: mol_type = genomic DNA
aacctacaaggttaaaat

SEQ ID NO: 62 - DNA, 20 bp, Homo sapiens
  source 1..20: mol_type = genomic DNA
caagaattacggatgagaaa

SEQ ID NO: 63 - DNA, 20 bp, Homo sapiens
  source 1..20: mol_type = genomic DNA
gtggaggagccagatattaa

SEQ ID NO: 64 - DNA, 20 bp, Homo sapiens
  source 1..20: mol_type = genomic DNA
aaaaaaaaatgtgttttcag

SEQ ID NO: 65 - DNA, 20 bp, Homo sapiens
  source 1..20: mol_type = genomic DNA
tttgttcaatggccaatcac

SEQ ID NO: 66 - DNA, 20 bp, Homo sapiens
  source 1..20: mol_type = genomic DNA
gaaaccccatctctactaaa

SEQ ID NO: 67 - DNA, 20 bp, Homo sapiens
  source 1..20: mol_type = genomic DNA
aggagcatgatacatgaata

SEQ ID NO: 68 - DNA, 20 bp, Homo sapiens
  source 1..20: mol_type = genomic DNA
ctcaaaaatccattgcaagt

SEQ ID NO: 69 - DNA, 20 bp, Homo sapiens
  source 1..20: mol_type = genomic DNA
gaccatctctgacatgaatc

SEQ ID NO: 70 - DNA, 20 bp, Homo sapiens
  source 1..20: mol_type = genomic DNA
gttaaaatctgaaaatgcag

SEQ ID NO: 71 - DNA, 20 bp, Homo sapiens
  source 1..20: mol_type = genomic DNA
cttgtgggcggtcatacaaa

SEQ ID NO: 72 - DNA, 18 bp, Homo sapiens
  source 1..18: mol_type = genomic DNA
caaaagagccaaacctga

SEQ ID NO: 73 - DNA, 19 bp, Homo sapiens
  source 1..19: mol_type = genomic DNA
caaaccaaacggacctgtc

SEQ ID NO: 74 - DNA, 20 bp, Homo sapiens
  source 1..20: mol_type = genomic DNA
cagatgagggaatttatctc

SEQ ID NO: 75 - DNA, 20 bp, Homo sapiens
  source 1..20: mol_type = genomic DNA
tacactgatgctaatgagct

SEQ ID NO: 76 - DNA, 20 bp, Homo sapiens
  source 1..20: mol_type = genomic DNA
acaagatgatgccatgtact

SEQ ID NO: 77 - DNA, 20 bp, Homo sapiens
  source 1..20: mol_type = genomic DNA
atatttattgaacatatagt

SEQ ID NO: 78 - DNA, 20 bp, Homo sapiens
  source 1..20: mol_type = genomic DNA
ccataaaggggactttttaa

SEQ ID NO: 79 - DNA, 20 bp, Homo sapiens
  source 1..20: mol_type = genomic DNA
aagcatttgcaaaggcgact

SEQ ID NO: 80 - DNA, 20 bp, Homo sapiens
  source 1..20: mol_type = genomic DNA
ttattttctaaattgtactg

SEQ ID NO: 81 - DNA, 20 bp, Homo sapiens
  source 1..20: mol_type = genomic DNA
ccccccaaaaacacagaatt

SEQ ID NO: 82 - DNA, 20 bp, Homo sapiens
  source 1..20: mol_type = genomic DNA
tccataacatggcagttgat

SEQ ID NO: 83 - DNA, 20 bp, Homo sapiens
  source 1..20: mol_type = genomic DNA
gtgagctacgttcctgcttc

SEQ ID NO: 84 - DNA, 20 bp, Homo sapiens
  source 1..20: mol_type = genomic DNA
taattttactaatcggattc

SEQ ID NO: 85 - DNA, 26 bp, Homo sapiens
  source 1..26: mol_type = genomic DNA
catcaactagtatgtgctatattccc

SEQ ID NO: 86 - DNA, 20 bp, Homo sapiens
  source 1..20: mol_type = genomic DNA
gtttgggtacaccaagtttt

SEQ ID NO: 87 - DNA, 20 bp, Homo sapiens
  source 1..20: mol_type = genomic DNA
acaggtgctagagaggatgt

SEQ ID NO: 88 - DNA, 20 bp, Homo sapiens
  source 1..20: mol_type = genomic DNA
aacctacaaggggttaaaat

SEQ ID NO: 89 - DNA, 20 bp, Homo sapiens
  source 1..20: mol_type = genomic DNA
caagaattatggatgagaaa

SEQ ID NO: 90 - DNA, 20 bp, Homo sapiens
  source 1..20: mol_type = genomic DNA
gtggaggaggcagatattaa

SEQ ID NO: 91 - DNA, 20 bp, Homo sapiens
  source 1..20: mol_type = genomic DNA
aaaaaaaaaagtgttttcag

SEQ ID NO: 92 - DNA, 20 bp, Homo sapiens
  source 1..20: mol_type = genomic DNA
tttgttcaaaggccaatcac

SEQ ID NO: 93 - DNA, 20 bp, Homo sapiens
  source 1..20: mol_type = genomic DNA
gaaaccccacctctactaaa

SEQ ID NO: 94 - DNA, 20 bp, Homo sapiens
  source 1..20: mol_type = genomic DNA
aggagcatgttacatgaata

SEQ ID NO: 95 - DNA, 20 bp, Homo sapiens
  source 1..20: mol_type = genomic DNA
ctcaaaaatccattgcaagt

SEQ ID NO: 96 - DNA, 23 bp, synthetic construct
  source 1..23: mol_type = other DNA
gggcacagtcctcaggtttgngg

SEQ ID NO: 97 - DNA, 23 bp, synthetic construct
  source 1..23: mol_type = other DNA
ggngtccaaaccccgagaaaaca

SEQ ID NO: 98 - DNA, 23 bp, synthetic construct
  source 1..23: mol_type = other DNA
ggngtccaaaccgagaaaacata

SEQ ID NO: 99 - DNA, 45 bp, Homo sapiens
  source 1..45: mol_type = genomic DNA
actcagggcacagtcctcaggtttggggctcttttgtatgactgc

SEQ ID NO: 100 - DNA, 45 bp, Homo sapiens
  source 1..45: mol_type = genomic DNA
gcagtcatacaaaagagccccaaacctgaggactgtgccctgagt

SEQ ID NO: 101 - DNA, 43 bp, Homo sapiens
  source 1..43: mol_type = genomic DNA
actcagggcacagtcctcaggtttggctcttttgtatgactgc

SEQ ID NO: 102 - DNA, 43 bp, Homo sapiens
  source 1..43: mol_type = genomic DNA
gcagtcatacaaaagagccaaacctgaggactgtgccctgagt

SEQ ID NO: 103 - DNA, 20 bp, synthetic construct
  source 1..20: mol_type = other DNA
gggcacagtcctcaggtttg

SEQ ID NO: 104 - DNA, 20 bp, synthetic construct
  source 1..20: mol_type = other DNA
acaaaagagccccaaacctg

SEQ ID NO: 105 - DNA, 20 bp, synthetic construct
  source 1..20: mol_type = other DNA
atacaaaagagccaaacctg

SEQ ID NO: 106 - DNA, 3315 bp, synthetic construct
  source 1..3315: mol_type = other DNA
atgggacccaagaaaaaacgcaaggtgggaagcggatccgccaaaaacaccattaccaaaacactgaaactgcgtattgtgcgtccgtataatagcgcagaagtggaaaaaattgttgccgacgaaaaaaacaaccgcgaaaaaatcgcactggaaaagaacaaagacaaagtgaaagaagcctgcagcaaacatctgaaagttgcagcatattgtaccacacaggttgaacgtaatgcatgcctgttttgtaaagcacgtaaactggatgacaaattctaccaaaaactgcgtggtcagtttccggatgcagttttttggcaagaaatcagcgaaatttttcgccagctgcagaaacaggcagcagaaatctataatcagagcctgatcgaactgtactacgagatttttatcaaaggcaaaggtattgcaaatgccagcagcgttgaacattatctgagtagagtttgttatagacgtgcagcagaactgtttaaaaacgcagcaattgcaagcggtctgcgtagcaaaatcaaaagcaattttcgtctgaaagaactgaaaaacatgaaaagtggtctgccgaccaccaaaagcgataattttccgattccgctggttaaacagaaaggtggtcagtataccggttttgaaattagcaatcataatagcgacttcatcatcaagattccgtttggtcgttggcaggtcaaaaaagagattgataaatatcgtccgtgggagaaatttgactttgaacaggttcagaaaagcccgaaaccgattagcctgctgctgagcacccagcgtcgtaaacgtaataaaggttggagcaaagatgaaggcaccgaagccgaaatcaaaaaagttatgaatggcgattatcagaccagctacattgaagttaaacgtggcagcaaaatctgtgaaaaaagcgcatggatgctgaatctgagcattgatgttccgaaaattgataaaggtgtggatccgagcattattggtggtattgcagttggtgttagatcaccgctggtttgcgcaattaacaatgcatttagccgttatagcatcagcgataacgacctgtttcacttcaacaagaaaatgtttgcacgtcgtcgtatcctgctgaaaaaaaaccgtcataaacgtgcaggtcatggtgcaaaaaacaaactgaaaccgatcaccattctgaccgaaaaaagtgaacgttttcgcaaaaagctgattgaacgttgggcatgtgaaatcgcggatttcttcattaaaaacaaagttggcaccgtgcagatggaaaatctggaaagcatgaaacgtaaagaggacagctattttaacattcgcctgcgtggcttttggccgtatgcagaaatgcagaacaaaatcgaattcaaactgaagcagtatggcatcgaaattcgtaaagttgcaccgaataataccagcaaaacctgtagcaaatgtggccatctgaacaactatttcaacttcgagtaccgcaagaaaaacaaattcccgcactttaaatgcgaaaaatgcaacttcaaagaaaacgccgcgtataatgcagccctgaatatttcaaacccgaaactgaaaagcaccaaagagagaccggcctatccctatgacgtgcccgattatgccagcctgggcagcggcgacggcattggtagtgggagcaacggcagcagcctcgatgctttagacgattttgacttagatatgcttggttcagacgcgttagacgacttcgacctagacatgttaggctcagatgcattggacgacttcgatttagatatgttgggctccgatgccctagatgactttgatctagatatgctaggtagtggcggcagcggatcccagtatctgcccgacacagatgatagacaccgaatcgaagagaaacgcaagcgaacgtatgaaaccttcaaatcgatcatgaagaaatcgcccttctcgggtccgaccgatcccaggcccccaccgagaaggattgcggtcccgtcccgctcgtcggccagcgtgccgaagcctgcgccgcagccctaccccttcacgtcgagcctgagcacaatcaattatgacgagttcccgacgatggtgttcccctcgggacaaatctcacaagcctcggcgctcgcaccagcgcctccccaagtccttccgcaagcgcctgccccagcgcctgcaccggcaatggtgtccgccctcgcacaggcccctgcgcccgtccccgtgctcgcgcctggaccgccccaggcggtcgctccaccggctccgaagccgacgcaggccggagagggaacactctccgaagcacttcttcaactccagtttgatgacgaggatcttggagcactccttggaaactcgacagaccctgcggtgtttaccgacctcgcgtcagtagataactccgaatttcagcagcttttgaaccagggtatcccggtcgcgccacatacaacggagcccatgttgatggaataccccgaagcaatcacgagacttgtgacgggagcgcagcggcctcccgatcccgcacccgcacctttgggggcacctggcctccctaacggacttttgagcggcgacgaggatttctcctccatcgccgatatggatttctcagccttgctgtcacagatttccagcggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttcatatgggtggaggctccggggaagatcctgctgccaaacgcgttaaactagactga

SEQ ID NO: 107 - DNA, 23 bp, synthetic construct
  source 1..23: mol_type = other DNA
cgaatacgcccacgcgatgggta

SEQ ID NO: 108 - DNA, 23 bp, synthetic construct
  source 1..23: mol_type = other DNA
gcactgtcaatcagaagaacagg

SEQ ID NO: 109 - DNA, 477 bp, synthetic construct
  source 1..477: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggcttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaatgaaggaatgcaacnnnnnnnnnnnnnnnnnnnnnnntttttttaagcttgggccgctcgagggatccattaggcggccgcg

SEQ ID NO: 110 - protein, 297 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

(19) United States
(12) Patent Application Publication
(10) Pub. No.: US 2025/0268992 A1
(43) Pub. Date: Aug. 28, 2025

(54) METHODS FOR TREATING DILATED CARDIOMYOPATHY AND PHARMACEUTICAL COMPOSITIONS THEREFOR

(71) Applicants: THE JACKSON LABORATORY, Bar Harbor, ME (US); UNIVERSITY OF CONNECTICUT, Farmington, CT (US)

(72) Inventors: John Travis HINSON, Farmington, CT (US); Shahnaz GHAHREMANI, Farmington, CT (US); Ketan THAKAR, Farmington, CT (US)

(21) Appl. No.: 18/859,795

(22) PCT Filed: Apr. 25, 2023
(86) PCT No.: PCT/US2023/066217

§ 371 Date: Oct. 24, 2024

Related U.S. Application Data
(60) Provisional application No. 63/334,539, filed on Apr. 25, 2022.

(51) Int. Cl. A61K 38/46; A61K 31/7088; A61P 9/04; C12N 9/22; C12N 15/11

(52) U.S. Cl. CPC A61K 38/465; A61K 31/7088; A61P 9/04; C12N 9/22; C12N 15/11

(57) ABSTRACT

This invention relates to genetic methods for altering gene expression in a human or animal. In particular, the invention relates to increasing expression of the TTN gene in a cell or tissue in an animal or human wherein at least one allele of the gene carries a mutation that results in a truncated Titin protein product. Therapeutic treatments, pharmaceutical compositions and methods of treatment utilizing such pharmaceutical compositions are also provided.

### CROSS-REFERENCE TO RELATED APPLICATIONS

This application claims priority to U.S. provisional application No. 63/334,539, filed Apr. 25, 2022, the disclosure of which is expressly incorporated by reference herein.

### INCORPORATION BY REFERENCE OF SEQUENCE LISTING PROVIDED ELECTRONICALLY

This application contains a Sequence Listing submitted as an electronic text file named “22-0135-WO.xml,” having a size in bytes of 105 kb, and created on Apr. 24, 2023. The information contained in this electronic file is hereby incorporated by reference in its entirety.

### TECHNICAL FIELD

This invention relates to genetic methods for altering gene expression in a human or animal. In particular, the invention relates to increasing expression of the TTN gene in a cell or tissue in an animal or human wherein at least one allele of the gene carries a mutation that results in a dysfunctional protein product, particularly a truncated Titin protein product. Pharmaceutical compositions and therapeutic treatment methods using the pharmaceutical compositions are also provided.

### BACKGROUND OF THE INVENTION

Dilated cardiomyopathy (DCM) is a prevalent condition occurring in 1:200 individuals that is associated with high morbidity and mortality despite current therapeutics and cardiac transplantation (Hershberger et al., 2013, Nat Rev Cadiol 10: 531-547). DCM is diagnosed by diminished cardiac ejection fraction and dilation of the left ventricle (Mestroni et al., 1999, Eur. Heart J 20: 93-102), and can have acquired or genetic causes or a combination of both factors (Japp et al., 2016, J Amer Coll Cardiol. 67: 2996-3010). Cataly zed by a rapid expansion in clinical genetic testing, pathogenic variants in DCM-associated genes can be identified in 17-26% of DCM individuals (Mazzaratto et al., 2020, Circulation 141: 387-398).

Among DCM-associated variants, titin (TTN) gene variants that result in premature protein truncations (TTNtv), such as nonsense, frameshift or splicing variants, are the most frequently identified genetic lesion found in DCM individuals (Roberts et al., 2015, Sci Trnsl Med 7: 270). In addition, TTNtvs have also been implicated through gene-environment interaction studies in acquired forms of heart failure such as peripartum cardiomyopathy (Ware et al., 2016, N. Engl. J. Med. 374: 233-241) and alcoholic cardiomyopathy (Ware et al., 2018, J. Am. Coll. Cardiol. 71: 2293-2302) in which the co-inheritance of a TTNtv allele along with environmental stress synergistically increases the risk for DCM. The TTN genetic locus also plays a modifier role in DCM as demonstrated by genome wide association studies (GWAS) that have associated common genetic variants near TTN with increased DCM risk (Tadros et al., 2021, Nat. Genet. 53: 128-134), and for DCM-like changes in cardiac structure (e.g., enlargement of cardiac chambers) and function (e.g. reduction in ejection fraction) in a healthy population study (Pirruccello et al., 2020, Nat. Commun. 11: 2254). Taken together, these human genetics studies have robustly demonstrated that inheritance of both rare and common variants that disrupt TTN functions are a major risk factor for DCM, but currently there are no treatments available in the clinic that restore TTN functions.

The lack of treatments targeting TTN partly reflects an incomplete knowledge of how TTN variants cause DCM. Historically, studying TTN functions has been challenging for the cardiac field because of TTN's immense size (e.g., TTN N2BA isoform is 34,350 residues (https://varsome.com/transcript/hg19/ENST00000591111.1), which is compounded by the lack of a robust TTNtv animal model that resembles human cardiac physiology. Recently, biomimetic 3-dimensional TTNtv microtissue models composed of human induced pluripotent stem cell-derived cardiomyocytes (“CMs”) have been developed that exhibit robust DCM phenotypes including diminished contractile function and myofibril content resembling features observed in human DCM hearts (Hinson et al., 2015, Science 349: 982-986). Cardiac microtissue models exhibit additional translational advantages relative to animal models such as the use of human cells expressing human sarcomere components, and human genetic sequences in which therapeutics that target human TTN can be efficiently developed and translated to humans. As proof of this principle, a recent study has demonstrated that a DCM-associated TTNtv can be functionally repaired using Clustered Regularly Interspaced Short Palindromic Repeats (CRISPR)/Cas9 technology (Romano et al., 2022, Circulation 145: 194-205). This method works by treatment of heterozygous TTNtv cardiac microtissues with Cas9 derived from Streptococcus pyogenes and a custom guide RNA (gRNA) that is programmed to bind and cut only the TTNtv allele. These results demonstrated that CMs repair the DNA double-strand break using endogenous repair processes that result in restoration of the TTN reading frame, and normalization of TTN protein levels and functions in cardiac microtissue functional assays. While this approach was successful for functional restoration of a single TTNtv identified in a DCM family, secondary to the extreme rarity of individual TTNtvs in the DCM population, this specific CRISPR treatment would not be generalizable to other DCM individuals that have different TTNtvs.

Thus there remains a need in this art for therapeutic interventions, methods, and pharmaceutical compositions directed to restoring TTN gene function in DCM patients.

### SUMMARY OF THE INVENTION

This invention provides therapeutic methods and pharmaceutical compositions directed to restoring TTN gene function in dilated cardiomyopathy (DCM) patients that can be generalizable to a large proportion of DCM individuals.

This invention provides methods for ameliorating dilated cardiomyopathy (DCM) in a subject which can be a human or animal in need thereof. In certain embodiments, these methods comprise delivering to target tissue in the human or animal a therapeutically effective amount of a composition capable of introducing a transcriptional activator at a site specific for a regulatory sequence controlling or affecting TTN gene expression, wherein expression of a functional TTN gene product is increased in the human's or animal's heart tissue or skeletal muscle tissue. In particular these methods can be used in subjects wherein DCM in the subject is the result of one TTN allele in the subject's genomic DNA encoding a TTN gene that produces a dysfunctional Titin protein gene product. In certain embodiments, the TTN allele encodes a truncated mutation, a nonsense mutation, a frameshift mutation, or a splice variant mutation of the TTN gene. In certain embodiments, the TTN allele encodes a genetic variant that reduces TTN expression levels. In certain embodiments, the target tissue is heart tissue or skeletal muscle tissue. In the methods of the invention, the composition is advantageously delivered to target tissue as a CRISPR-Cas9 complex. In these embodiments the CRISPR-Cas9 complex comprises Cas9 protein wherein the nuclease activity is reduced or ablated (termed herein “a nuclease-dead Cas9 protein”). Further in these embodiments, the nuclease-dead Cas9 protein is linked to a eukaryotic transcriptional activator protein and associated with a guide RNA specific for a regulatory sequence controlling or affecting TTN gene expression. In these embodiments, the guide RNA molecule is identified by any of SEQ ID NO: 9, SEQ ID NOs: 13-21, SEQ ID NOs: 23-25, SEQ ID NOs: 27-32, SEQ ID NO: 33, SEQ ID NOs: 35-37, and SEQ ID NOs: 38-41. In specific embodiments, the activator protein is VPR (termed herein dCas9-VPR). In alternative specific embodiments, the activator protein is VP64R (termed herein dCas9-VP64). In further specific embodiments, the activator protein is SunTag (termed herein dCas9-SunTag). In still further specific embodiments, the activator protein is SAM (termed herein dCas9-SAM). In specific embodiments, the regulatory sequence controlling or affecting TTN gene expression is located within TTN gene promoter region. In certain embodiments, the guide RNA targeting the TTN gene promoter region is a sgRNA having a sequence identified by any one of SEQ ID NO: 9, SEQ ID NOs: 13-21, SEQ ID NOs: 23-25, SEQ ID NOs: 27-32, and SEQ ID NOs: 38-41. In certain advantageous embodiments, the guide RNA targeting the TTN gene promoter region is a sgRNA having a sequence identified by any one of SEQ ID NOs: 13, 14, 15, 21, 24, 25, 27, 28, 30, 31, and 38-41. In specific embodiments, the regulatory sequence for TTN gene expression is located within TTN gene enhancer region. In certain embodiments, the guide RNA targeting the TTN gene enhancer region is a sgRNA having a sequence identified by any one of SEQ ID NO: 33, and SEQ ID NOs: 35-37. In certain advantageous embodiments, the guide RNA targeting the TTN gene enhancer region is a sgRNA having a sequence identified by SEQ ID NO: 33.

In certain embodiments, the CRISPR-Cas9 complex delivered to heart tissue or skeletal muscle tissue in the subject is delivered by one or a plurality of expression constructs encoding the nuclease-dead Cas9 protein linked to an activator protein and a guide RNA specific for regulatory sequences for TTN gene expression.

The invention also provides compositions, in particular therapeutic compositions, for restoring TTN gene function in DCM patients that can be generalizable to a large proportion of DCM individuals. In certain embodiment, the compositions are pharmaceutical compositions. In particular embodiments, the invention provides therapeutic compositions and pharmaceutical compositions comprising a CRISPR-Cas9 complex. In these embodiments the CRISPR-Cas9 complex comprises a nuclease-dead Cas9 protein. Further in these embodiments, the nuclease-dead Cas9 protein is linked to a eukaryotic transcriptional activator protein and associated with a guide RNA specific for a regulatory sequence controlling or affecting TTN gene expression. In these embodiments the CRISPR-Cas9 complex comprises Cas9 protein wherein the nuclease activity is reduced or ablated (termed herein “a nuclease-dead Cas9 protein”). Further in these embodiments, the nuclease-dead Cas9 protein is linked to a eukaryotic transcriptional activator protein and associated with a guide RNA specific for a regulatory sequence controlling or affecting TTN gene expression. In these embodiments, the pharmaceutical compositions further comprises the guide RNA molecule identified by any of SEQ ID NO: 9, SEQ ID NOs: 13-21, SEQ ID NOs: 23-25, SEQ ID NOs: 27-32, SEQ ID NO: 33, SEQ ID NOs: 35-37, and SEQ ID NOs: 38-41. In specific embodiments, the activator protein is VPR (termed herein dCas9-VPR). In alternative specific embodiments, the activator protein is VP64R (termed herein dCas9-VP64). In further specific embodiments, the activator protein is SunTag (termed herein dCas9-SunTag). In still further specific embodiments, the activator protein is SAM (termed herein dCas9-SAM). In specific embodiments, the CRISPR-Cas9 complex comprises dCas9-VPR. In specific embodiments, the regulatory sequence controlling or affecting TTN gene expression is located within TTN gene promoter region. In certain embodiments, the guide RNA targeting the TTN gene promoter region is a sgRNA having a sequence identified by any one of SEQ ID NO: 9, SEQ ID NOs: 13-21, SEQ ID NOs: 23-25, SEQ ID NOs: 27-32, and SEQ ID NOs: 38-41. In certain advantageous embodiments, the guide RNA targeting the TTN gene promoter region is a sgRNA having a sequence identified by any one of SEQ ID NOs: 13, 14, 15, 21, 24, 25, 27, 28, 30, 31, and 38-41. In specific embodiments, the regulatory sequence for TTN gene expression is located within TTN gene enhancer region. In certain embodiments, the guide RNA targeting the TTN gene enhancer region is a sgRNA having a sequence identified by any one of SEQ ID NO: 33, and SEQ ID NOs: 35-37. In certain advantageous embodiments, the guide RNA targeting the TTN gene enhancer region is a sgRNA having a sequence identified by SEQ ID NO: 33.

In certain embodiments, the CRISPR-Cas9 complex delivered to heart tissue or skeletal muscle tissue in the subject is delivered by one or a plurality of expression constructs encoding the nuclease-dead Cas9 protein linked to an activator protein and a guide RNA specific for regulatory sequences for TTN gene expression.

This invention provides methods for ameliorating dilated cardiomyopathy (DCM) in a subject which can be a human or animal in need thereof. In certain embodiments, these methods comprise delivering to target tissue in the human or animal a therapeutically effective amount of a composition capable of introducing a transcriptional activator at a site specific for a regulatory sequence controlling or affecting a wild type TTN allele that produces a functional Titin protein gene product, wherein expression of the functional Titin protein is specifically increased in the subject's heart tissue or skeletal muscle tissue. In particular, these methods can be used in subjects wherein DCM in the subject is the result of a mutated TTN allele in the individual's genomic DNA encoding a TTN gene that produces a dysfunctional Titin protein gene product. In certain embodiments, the TTN allele encodes a truncated mutation, a nonsense mutation, a frameshift mutation, or a splice variant mutation of the TTN gene. In certain embodiments, the TTN allele encodes a genetic variant that reduces TTN expression levels. In certain embodiments, the target tissue is heart tissue or skeletal muscle tissue. In the methods of the invention, the composition is advantageously delivered to target tissue as a CRISPR-Cas9 complex. In these embodiments the CRISPR-Cas9 complex comprises Cas9 protein wherein the nuclease activity is reduced or ablated (termed herein “a nuclease-dead Cas9 protein”). Further in these embodiments, the nuclease-dead Cas9 protein is linked to a eukaryotic transcriptional activator protein and associated with a guide RNA specific for a regulatory sequence controlling or affecting the wild type TTN allele gene expression. In specific embodiments, the activator protein is VPR (termed herein dCas9-VPR). In alternative specific embodiments, the activator protein is VP64R (termed herein dCas9-VP64). In further specific embodiments, the activator protein is SunTag (termed herein dCas9-SunTag). In still further specific embodiments, the activator protein is SAM (termed herein dCas9-SAM). In specific embodiments, the regulatory sequence controlling or affecting the TTN gene expression is located within the wild type TTN allele gene promoter region or the wild type TTN allele gene enhancer region. In certain embodiments, the guide RNA targeting the wild type TTN allele gene promoter region or the wild type TTN allele gene enhancer region is a sgRNA having a sequence identified by any one of SEQ ID NOs: 103-105.

In certain embodiments, the CRISPR-Cas9 complex delivered to heart tissue or skeletal muscle tissue in the subject is delivered by one or a plurality of expression constructs encoding the nuclease-dead Cas9 protein linked to an activator protein and a guide RNA specific for regulatory sequences for the wild type TTN allele gene expression.

The invention also provides compositions, in particular therapeutic compositions, for restoring TTN gene function in DCM patients that can be generalizable to a large proportion of DCM individuals. In certain embodiments, the compositions are pharmaceutical compositions. In particular embodiments the invention provides therapeutic compositions and pharmaceutical compositions comprising a CRISPR-Cas9 complex. In these embodiments the CRISPR-Cas9 complex comprises a nuclease-dead Cas9 protein. Further in these embodiments the nuclease-dead Cas9 protein is linked to a eukaryotic transcriptional activator protein and associated with a guide RNA specific for a regulatory sequence controlling or affecting the wild type TTN allele gene expression. In these embodiments the CRISPR-Cas9 complex comprises Cas9 protein wherein the nuclease activity is reduced or ablated (termed herein “a nuclease-dead Cas9 protein”). Further in these embodiments, the nuclease-dead Cas9 protein is linked to a eukaryotic transcriptional activator protein and associated with a guide RNA specific for a regulatory sequence controlling or affecting the wild type TTN allele gene expression. In these embodiments, the pharmaceutical compositions further comprises the guide RNA molecule identified by any of SEQ ID NOs: 103-105. In specific embodiments, the activator protein is VPR (termed herein dCas9-VPR). In alternative specific embodiments, the activator protein is VP64R (termed herein dCas9-VP64). In further specific embodiments, the activator protein is SunTag (termed herein dCas9-SunTag). In still further specific embodiments, the activator protein is SAM (termed herein dCas9-SAM). In specific embodiments, the regulatory sequence controlling or affecting TTN gene expression is located within the wild type TTN allele gene promoter region or the wild type TTN allele gene enhancer region. In these embodiments, the pharmaceutical compositions further comprises the guide RNA molecule targeting the wild type TTN allele gene promoter region or the wild type TTN allele gene enhancer region identified by any of SEQ ID NOs: 103-105.

In certain embodiments, the CRISPR-Cas9 complex delivered to heart tissue or skeletal muscle tissue in the subject is delivered by one or a plurality of expression constructs encoding the nuclease-dead Cas9 protein linked to an activator protein and a guide RNA specific for regulatory sequences for the wild type TTN allele gene expression.

Therapeutic compositions and pharmaceutical compositions of the invention comprise CRISPR-Cas9 complex to be delivered to heart tissue or skeletal muscle tissue in the individual that is advantageously an intact CRISPR-Cas9 complex comprising the nuclease-dead Cas9 protein linked to an activator protein and a guide RNA specific for regulatory sequences for TTN gene expression or for the wild type TTN allele gene expression. Therapeutic compositions and pharmaceutical compositions of this invention can be formulated in lipid nanoparticles, lentivirus constructs, or adenovirus or adeno-associated virus constructs.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

FIG. 1A to FIG. 1G illustrate the results of experiments using human cardiac microtissue and mouse models harboring TTNtvA to develop DCM therapeutics. FIG. 1A provides a schematic diagram providing a general overview of TTNtvA iPSc model production using CRISPR/Cas9 technology to introduce a heterozygous (+/−) truncation variant at residue 22582fs within the TTN A-band domain (TTN isoforms commonly expressed in CMs are shown including N2BA (Q8WZ42-1), Novex3 (Q8WZ42-6) and Cronos. TTN structural domains are indicated by color in left-to-right order including red (Z-disk; encoded by chr2:179672150-179640083 from human genome assembly GRCh37(hg19)), blue (I-band; encoded by chr2: 179639929-179483311 from human genome assembly GRCh37(hg19)), green (A-band; encoded by chr2:179483218-179400709 from human genome assembly GRCh37(hg19), and purple (M-line; encoded by chr2: 100766-107976 from human genome assembly GRCh37(hg19)). FIG. 1B similarly provides a schematic diagram providing a general overview of a cardiac microtissue assay to measure twitch force or contractility using cantilever displacement analysis. Cardiac microtissues are generated from human CMs, human cardiac fibroblasts and collagen. White arrows denote direction of cantilever displacement with twitch. FIG. 1C shows cardiac microtissues from TTNtvA^{+/-} (blue bar, right) and wildtype (WT; gray bar, left) control conditions (each black circle represents a single cardiac microtissue generated from at least biological triplicates by differentiation batch) were analyzed for maximum twitch force generation. FIG. 1D shows the results of VAGE profiling of TTN protein isoform expression from lysates obtained from wildtype (WT) and TTNtvA^{+/-} CMs. Red arrows denote truncated TTN protein (tv) of the size expected for truncated N2BA isoform (N2BAtv). SYPRO Ruby stain was used to visualize proteins. FIG. 1E shows quantification of full-length N2BA protein and FIG. 1F shows the results obtained using truncated N2BA (N2BAtv) from lysates obtained from WT and TTNtvA^{+/-} CMs (with results from WT TTN on the left and TTNtvA^{+/-} CMs on the right in each bar graph). FIG. 1G is a schematic diagram of TTNtvA knock-in mouse model production using CRISPR/Cas9 technology to introduce a heterozygous (+/−) truncation variant near residue 22582 including a 33 kDa HaloTag® (SEQ ID NO: 110) proximal to the STOP codon to provide a method to discriminate all truncated TTN products obtained from TTNtvA. FIG. 1H illustrates a representative example of VAGE profiling of TTN expression by immunoblotting (WB) using anti-Z TTN antibody (red Y) or anti-HaloTag® antibody (gray Y) from lysates obtained from WT and TTNtvA-HaloTag®+/− hearts. The red arrow denotes truncated TTN doublet of the size expected for truncated N2BA and N2B isoforms. Data are set forth as the mean±SD; significance is assessed by ordinary one-way ANOVA with consecutive Dunnett's multiple comparison test using WT as control (C, E, F) and defined by P<0.05 (*), P≤0.01 (**), and P≤0.0001 (****).

FIG. 2A to FIG. 2F show methods and constructs used to study TTN transcriptional activation in treating TTNtv-related dilated cardiomyopathy. FIG. 2A provides a schematic diagram of a general overview of DCM pathogenesis due to TTNtv, including reductions in TTN protein and decreased contractile functions that can be reversed using dCas9-VPR to activate TTN transcriptional activity; this is achieved by increased TTN mRNA that leads to increased TTN protein levels and normalization of contractile functional deficits. FIG. 2B is an overview of CRISPR/Cas9-based methods to generate CMs wherein expression of CM-specific dCas9-VPR using a donor template to introduce T2A-dCas9-VPR into the TNNT2 locus is shown. The result is a robust CM platform to develop and refine the methods for TTN transcriptional activation. FIG. 2C sets forth the components within the donor template used for FIG. 2B. FIG. 2D shows a representative immunoblot from iPSc and CM lysates probed for antibodies to dCas9-VPR. MYH6/7, pan-ACTN, TNNT2, and GAPDH, demonstrating CM-specific expression of cleaved dCas9-VPR and cleaved TNNT2. FIG. 2E is a schematic diagram illustrating a CRISPR/Cas9 method to generate CMs for use in a quantitative assay for measuring TTN protein levels using insertion of tdTomato into the TTN locus in CMs. FIG. 2F is a schematic diagram setting out the components within the donor template used for FIG. 2E.

FIG. 3A is a schematic diagram of a general overview of CRISPRa in TTNtv+/− CMs that utilizes a nuclease dead Cas9 fused to VP64, p65 and Rta (dCas9-VPR) targeted to gene promoters by single guide RNAs to enable transcriptional activation. These dCas9-VPR-TTNtv+/− CMs have dCas9-VPR knocked into the TNNT2 locus to provide a method for CM-specific dCas9-VPR expression and following transduction with lentiviral vectors encoding single gRNAs targeted to gene promoters, can be utilized to study the functional consequences of CRISPRa in a DCM context. FIG. 3B shows ATAC-seq peak analysis of TTN N2BA promoter from four CM samples. The N2BA transcript is NM 001256850. FIG. 3C shows the results of quantitative PCR analysis of TTNmRNA activation after lentiviral transduction of a single gRNA targeting the TTN N2BA promoter or non-targeting (NT) controls in the dCas9-VPR CM background, demonstrating activation of splice isoforms containing Z-disk, Novex3 and A-band TTN transcripts. FIG. 3D shows the results of a representative FACS gating strategy to illustrate the assay utilized for TTN-tdTomato protein level quantification in CMs. Debris is excluded from cells using FSC (forward scatter) vs SSC (side scatter) gating, dead cells are excluded from viable cells using To-pro-3 staining, and TTN-tdTomato is measured in the total population. FIG. 3E is a representative FACS histogram of TTN-tdTomato levels in CMs after TTN N2BA promoter activation at low and high multiplicity of infection (MOI) and compared to NT controls. The Figure shows dose-dependent TTN activation with a right-shift in TTN-tdTomato intensity at high MOI vs low MOI, and at low MOI vs NT controls.

FIG. 4A shows the results of single gRNAs (NPA-NPX) screening at two time points for TTN-tdTomato activation and normalized to NT controls. Values >1 are activating and values <1 are inhibiting TTN-tdTomato levels. FIG. 4B is an overview of TTN isoforms abundant in CMs and antibodies used to decorate isoforms in CM lysates. FIG. 4C is a representative VAGE immunoblot of TTN protein isoforms and levels after TTN promoter activation probed with Anti-Z TTN antibodies across three MOIs. N2BA, truncated TTN and Novex3 are all activated in a dose-dependent manner. Also, TTN band streaking is an indicator of overloading the lane. FIG. 4D shows VAGE immunoblot results obtained from CM lysates probed for TTN protein isoforms using an Anti-Z TTN antibody demonstrates that TTN promoter activation increases the protein levels of N2BA, truncated N2BA (N2BAtv) and Novex3 TTN isoforms. Prior to VAGE replicates were normalized to actinin levels. FIG. 4E is a quantification of VAGE immunoblot results from FIG. 4B using NPV (right) normalized to NT (left) gRNA. FIG. 4F is a representative VAGE immunoblot of TTN protein isoforms and levels after TTN promoter activation using NPV probed with anti-M TTN antibodies. N2BA, but not Cronos is activated, which is consistent with Cronos TTN's distinct internal promoter usage. FIG. 4G shows VAGE immunoblot results obtained from CM lysates probed for TTN protein isoforms using an Anti-M TTN antibody demonstrates that TTN promoter activation increases the protein levels of N2BA, but not Cronos that utilizes a distinct promoter. Prior to VAGE, replicates were normalized to actinin levels. FIG. 4H shows quantification of VAGE immunoblot results (from FIG. 4D) using NPV normalized to NT gRNA.

FIG. 5A provides schematic diagram of a general overview of cardiac microtissue assay composed of CMs, human cardiac fibroblasts and extracellular matrix composed of type I collagen. Using this assay, TTN transcriptional activation (NPE, right) results in increased twitch force compared to controls (NT, left). Each dot represents an independent microtissue replicate. FIG. 5B shows protein markers of the unfolded protein response (ATFa large isoform, IRE1α, phosphorylated EIF2α, total EIF2α) upon TTN transcriptional activation using NPV normalized to NT gRNAs as control. FIG. 5C are bar graphs showing quantification of immunoblots in FIG. 5B reveals no difference in the levels of unfolded protein response markers upon TTN transcriptional activation using NPV (right) normalized to NT (left) gRNAs as control.

FIG. 6A is a principal component analysis plot of RNA sequencing data obtained from biological CM triplicates treated with NPV or NT gRNA as controls. FIG. 6B is a pie chart summarizing differential gene expression (DGE) analysis following TTN transcriptional activation. DGE parameters included a false discovery rate-adjusted P value (P_{adj}) cutoff <0.05, which identified a total of 8219 genes. Of these, 7.01% were upregulated (Log₂ fold change (FC)≤1) and 16.52% were downregulated (Log₂ FC≤−1), while most were unchanged. FIG. 6C to FIG. 6E show volcano plots and Gene Ontology (GO) term enrichment analyses of the downregulated (blue; Log₂FC≤−1, P_{adj}<0.05; FIG. 6C, see list of gene names in FIG. 12A to FIG. 12C) and the upregulated (red; Log₂FC≥1, P_{adj}<0.05; FIG. 6D, see list of gene names in FIG. 13A to FIG. 13B) gene transcripts upon TTN transcriptional activation using NPV relative to NT gRNA controls. FIG. 6F is fold change heat maps of all gene transcripts within GO terms related to sarcomere structure and function reveals generalized upregulation (see corresponding FIG. 14A to FIG. 14B for gene names) including factors involved in cardiac myofibril assembly (GO: 0055003), cardiac muscle contraction (GO: 0060048), sarcomere organization (GO: 0045214), M-line (GO: 0031430), I-band (GO: 0031674), Z-disk (GO: 0030018), and A-band (GO: 0031672).

FIG. 7A to FIG. 7E show TTN transcriptional activation using TTN regulatory elements. FIG. 7A is a schematic diagram showing a general overview of methods for TTN transcriptional activation using dCas9-VPR directed by gRNAs to bind DNA regulatory element sequences, such as enhancers to activate TTN mRNA, TTN protein levels and normalize contractile function in DCM models. The diagram also depicts how CM-specific DNA-DNA contacts enable dCas9-VPR to physically access the TTN TSS and provide TTN transcriptional activation exclusively in CMs, but not iPSCs and likely other cell types. FIG. 7B shows ATAC-seq peaks from four CM samples at a TTN DNA regulatory element (E1-E5). Peaks are mapped to hg38 and coordinates are shown above peaks. FIG. 7C illustrates CM DNA-DNA loops shown using ChIA-PET using RNA polymerase II precipitation (RNAPII), and H3K27ac ChIP-seq peaks are shown near the N2BA TTN transcriptional start site (TSS). DNA regulatory elements are either looped to the TSS and/or are labeled by the active H3K27ac regulatory mark suggesting their function in TTN regulation. FIG. 7D shows single gRNAs (NEA-NEE) that were screened at two time points for TTN-tdTomato activation and normalized to NT controls. Values >1 are activating and values <1 are inhibiting TTN-tdTomato levels. FIG. 7E shows a representative VAGE immunoblot of TTN protein isoforms and levels after TTN E1 activation using NEA probed with Anti-Z TTN antibodies. N2BA, truncated TTN and Novex3 are all activated in a dose-dependent manner resembling changes associated with TTNN2BA promoter activation. Data are shown as mean±SD; significance was assessed by unpaired, two-tailed t-test with NT serving as control and defined by P≥0.05 (ns or not labeled) and P<0.05 (*).

FIG. 8A to FIG. 8D show the results of experiments using TTN isoform-specific transcriptional activation with an internal promoter. FIG. 8A provides a schematic diagram showing a general overview of methods of TTN transcriptional activation using dCas9-VPR directed by gRNAs to bind a TTN internal promoter to activate TTN mRNA, TTN protein levels and normalize contractile function in DCM models. FIG. 8B shows ATAC-seq peak analysis of TTN internal promoter from four CM samples. Peak is internal to N2BA transcript that is NM_001256850. FIG. 8C shows single gRNAs (CPA-CPE) that were screened for TTN-tdTomato activation and normalized to NT controls. Values >1 are activating and values <1 are inhibiting TTN-tdTomato levels. FIG. 8D is a representative VAGE immunoblot of TTN protein isoforms and levels after TTN internal promoter activation using CPA probed with Anti-M TTN antibodies. The Cronos isoform is activated, but not the N2BA TTN isoform. Data are shown as the mean+SD; significance was assessed by unpaired, two-tailed t-test with NT serving as control and defined by P≥0.05 (ns or not labeled) and P<0.05 (*).

FIG. 9A to FIG. 9G show TTN allele-specific transcriptional activation based on common genetic variation. FIG. 9A provides a schematic diagram showing a general overview of methods for allele-specific TTN transcriptional activation using dCas9-VPR directed by gRNAs to bind DNA sequences overlapping common genetic variants near the TTN promoter or other DNA regulatory elements wherein only normal TTN is activated but not truncated TTN (tv). Such methods are expected to increase normal TTN protein production in a target cell or tissue but not truncated TTN. FIG. 9B shows ATAC-seq peak analysis of the TTNN2BA promoter from four CM samples with common genetic variant rs72647838 shown for the major allele A (GGGG) and the minor allele B (GG- -). gRNAs rs72647838A and rs72647838A2 are a perfect match by Watson-Crick pairing to allele A, while rs72647838B is a perfect match to allele B. Because DCM patients with TTNtvs have one truncated allele and one normal allele, these common genetic variants can serve as a fingerprint landing pad for dCas9-VPR to active only the normal allele. FIG. 9C is a schematic diagram showing a general overview of TTN promoter luciferase assay for quantification of allele-specific TTN transcriptional activation leveraging rs72647838 allele A (GGGG). FIG. 9D shows TTN promoter (allele A) luciferase activity quantified by NanoGlo assay, demonstrating transcriptional activation only with transduction of dCas9-VPR and rs72647838A and rs72647838A2 gRNAs, but not rs72647838B gRNA. FIG. 9E is a general overview of TTN promoter luciferase assay for quantification of allele-specific TTN transcriptional activation directed to rs72647838 allele B (GG- -). FIG. 9F shows TTN promoter (allele B) luciferase activity quantified by NanoGlo assay, demonstrating transcriptional activation only with transduction of dCas9-VPR and rs72647838B gRNA, but not rs72647838A and rs72647838A2 gRNAs. FIG. 9G is a schematic diagram showing a general overview of method of TTN transcriptional activation using dCasMini-VPR directed by gRNAs to bind DNA sequences overlapping common genetic variants near the TTN promoter or other DNA regulatory elements such as promoter or other regulatory elements. FIG. 9H shows TTN promoter luciferase activity quantified by NanoGlo assay, demonstrating transcriptional activation with transduction of dCasMini-VPR and gRNA4 relative to NT controls. Data are displayed as mean±SD; statistical significance is assessed by unpaired, two-tailed t-test with NT serving as control and defined by P≥0.05 (ns or not labeled), P<0.05 (*) or P<0.005 (**).

FIG. 10A to FIG. 10E show TTN transcriptional activation restores TTNtv-induced sarcomere content and contractility deficits. FIG. 10A shows representative confocal images of CMs after TTN transcriptional activation using a NPV or NT gRNA as control. The sarcomere was stained using Anti-Z TTN antibody with DAPI co-stain for DNA. Note that CMs were patterned with fibronectin PDMS stamps to control cell shape and promote maturation. FIG. 10B shows quantification of sarcomere area after TTN transcriptional activation, wherein NPV results are shown in the righthand bar and NT are shown in the lefthand bar. FIG. 10C is a general overview of a 3-dimensional cardiac microtissue (CMT) assay to enable contractility (twitch force) measurements within a biomimetic context. CMTs are composed of CMs, human cardiac fibroblasts and an extracellular matrix (ECM) slurry, and twitch force is measured using cantilever displacement analysis at 1 Hz pacing. White arrows denote direction of cantilever displacement with twitch. FIG. 10D shows that TTN transcriptional activation using NPV (right bar) increased CMT twitch force relative to NT (left bar) gRNA as a control. FIG. 10E is a model to summarize that CRISPRa using dCas9-VPR targeted to TTN regulatory elements or promoters using single gRNAs restores changes in contractile function, sarcomere content and protein defects. As increasing TTN protein levels restores TTNtv-related dysfunction despite increasing truncated TTN protein levels, haploinsufficiency can be the predominant genetic mechanism for TTNtvs. Data are mean±SD; significance for pairwise comparison was assessed by Students T-test with Welch correction and defined by P<0.05 (*) and P≤0.01 (**).

FIG. 11A and FIG. 11B show that TTN/Ttn transcriptional activation reverses heart failure associated with Ttn truncation variants, chemical stressors (e.g., angiotensin II and isoproterenol), and pressure overload stress (transverse aortic banding or other hypertension models) alone or in combination.

FIG. 12A to FIG. 12C are lists of downregulated genes plotted in FIG. 6C to FIG. 6E. Downregulated genes filtered from RNA-seq data using parameters of false discovery rate26 adjusted P value (Padj)<0.05 and Log₂ FC≤−1.

FIG. 13A and FIG. 13B are lists of upregulated genes plotted in FIG. 6C to FIG. 6E. Upregulated genes filtered from RNA-seq data using parameters of false discovery rate-adjusted P value (P_{adj})<0.05 and Log₂ FC≥1.

FIG. 14A to FIG. 14B is the gene map legend corresponding to GO Terms in FIG. 6F.

### DETAILED DESCRIPTION OF THE INVENTION

Provided herewith is a more detailed description of the compositions, methods, and kits comprising the invention, which is provided to explain and enhance but not replace or be a substitute for the claims set forth below.

Unless defined otherwise, all technical and scientific terms used herein have the same meaning as is commonly understood by one of ordinary skill in the art. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). These references are intended to be exemplary and illustrative and not limiting as to the source of information known to the worker of ordinary skill in this art. All citations and references set forth herein are expressly incorporated in their entireties.

As used herein, the following terms have the meanings ascribed to them below, unless specified otherwise.

It is noted here that as used in this specification and the appended claims, the singular forms “a,” “an,” and “the” also include plural reference, unless the context clarity dictates otherwise.

The term “about” or “approximately” means within 25%, such as within 20% (or 5% or less) of a given value or range.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “transcription activation methodologies” is intended to include CRISPR and CRISPRa, TALENs (transcription activation-like effector nucleases; Cermak et al., 2011, Nucl. Acids Res. 39: e82) and TALE-TFs (transcription activation-like effector transcription factors; Sanjana et al., 2012, Nat. Protoc. 7: 171-192; Zhang et al., 2011, Nat. Biotechnol. 29: 149-153), ZFN (zinc finger nucleases; Porteus & Baltimore, 2003, Science 300: 763) and ZFN-TFs (zinc finger nuclease transcription factors; Beerli et al., 2000, Proc. Natd. Acad. Sci. USA 97: 1495-1500), as well as any protein comprising aprogrammable DNA binding domain combined with a transcriptional activator (see artificial transcription factors described in Ansari & Mapp, 2002, Curr. Opin. Chem. Biol. 6: 765-772), and any protein comprising a programmable DNA binding domain combined with an enzyme that activates transcription through epigenetic mechanisms such as histone acetyltransferases (e.g., dCas9-p300 as described in Klann et al., 2017, Nat. Biotechnol. 35: 561-568).

As used herein, the term “CRISPR” (Clustered Regularly Interspaced Short Palindromic Repeats) is intended to encompass all embodiments of targeted delivery of genetic or enzymatic modalities using a guide RNA encompassed in a protein, particularly a bacterial protein and most particularly said proteins related to CRISPR endonucleases with and without endonuclease activity, derived from bacterial species including but not limited to S. pyogenes and S. aureus, as disclosed in Jinek et al., 2012, Science 337: 816-21; Cong et al., 2013, Science 339: 819-823.

As used herein, the term “CRISPR endonuclease” is intended to encompass Cas9 from a number of bacterial species including S. pyogenes and S. aureus (termed “Type II” systems”), as well as Cas12a (termed “Type V systems”), Cas12f, CasMINI and CasΦ.

In particular embodiments, termed “CRISPRa,” the Cas9 protein, designated as “dCas9,” is a species of the Cas9 protein in which the endonuclease activity has been diminished or ablated but which retains the capacity to bind dual-molecule (tracrRNA and crRNA) or single-molecule (sgRNA, wherein the tracrRNA and crRNA are linked by an oligoribonucleotide linker) and to specifically target the CRISPR complex to a DNA sequence complementary to the crRNA sequence (Qi et al., 2013, Cell 152: 1173-1183). Accordingly, dCas9-CRISPR complexes can be used to deliver molecules, including transcription activators, to such sites. See, e.g., Bikard et al., 2013, Nucl. Acids Res. 41: 7429-7437; Perez-Pinera et al., 2013, Nat. Method 10: 973-976; Tannenbaum et al., 2014, Cell 159: 635-646; Konerman et al., 2014, Nature 517: 583-588; Chavez et al., 2015, Nat. Methods 12: 326-328; Riedmayr et al., 2022, Nature Protocols 17: 781-818.

As used herein, the term “transcription activator domains” is intended to encompass proteins capable of increasing transcription in genes having transcriptional regulatory elements responsive to such activators. See, Ma, 2011, Prot. & Cell 2: 879-888. In particular for uses as set forth herein to activate TTN gene expression and as part of a complex with dCas9, transcription activator domains include VPR (a tripartite complex of VP64, P65, and Rta; see, Chavez et al., 2015, Nat Methods 12: 326-328), VP64 (see, Casas-Mollano et al., 2020, The CRISPR J., https://doi.org/i0.1089/crispr.2020.0064), SunTag, (comprising multiple copies of VP64; see, Tanenbaum et al., 2014, Cell 159: 635-646), CBP (a histone acetyltransferase domain; Sajwan & Mannervik, 2019, Sci Rep. 9: 18104), Synergistic Activation Mediator (SAM); Zhang et al., Sci. Rep. 5: 16227), and SPH (a hybrid comprising the epitope tag of SunTag and the P65-HSF activation domains of SAM (see, Zhou et al., 2018, NatNeurosci 20: 440-446; Clouse, 2020, https://blog.addgene.org/crispr-activators-dcas9-vp64-sam-suntag-vpr; Chavez et al., 2016, Nat Methods 13: 563-567).

As used herein, the term “guide RNA” is intended to encompass dual-molecule embodiments (tracrRNA and crRNA) and single-molecule embodiments (sgRNA, wherein the tracrRNA and crRNA are linked by an oligoribonucleotide linker), capable of binding to bacterially derived Cas9 endonucleases (or inactivated embodiments thereof generally termed “dCas9”) and to specifically bind to a DNA sequence complementary to crRNA.

As used herein, “mutation” is intended to encompass point mutations including nonsense, missense, and frameshift mutations, as well as insertions, deletions, rearrangements, and splice site variants.

As used herein, “transcription start site” (TSS) means a location where the wild type DNA nucleotide is transcribed into RNA. As used herein, “promoter” is intended to encompass a region of DNA upstream of a gene where relevant proteins bind to initiate transcription of that gene. The promoter region exists upstream and downstream of the TSS. In some embodiments, gRNA to be used with dCas-9 for increasing TTN expression is designed to target promoter regions within 500 bp upstream (+500) and within 2000 bp downstream (−2000) of TSS.

As used herein, “enhancer” is intended to encompass DNA-regulatory elements that activate transcription of a gene or genes to higher levels than would be the case in their absence. These elements, such as cis-acting DNA regulatory elements, function at a distance by forming chromatin loops to bring the enhancer and target gene into proximity. Examples of such elements have been demonstrated to regulate expression of target genes (Wei et al., 2006, Cell 124: P 207-219; Li et al., 2020, Nature 11: 485).

As used herein, “target tissue” is intended to encompass any particular tissue wherein delivery of the TTN gene activating constructs set forth herein can be used advantageously for therapeutic purposes. In particular muscle tissue, especially skeletal muscle and most particularly cardiac or heart muscle tissue, is a target tissue as defined herein.

As used herein, targets for affecting gene expression include in particular genes encoding the Titin protein encoded by the TTN gene in humans or animals, particularly mammals and most particularly humans.

As used herein, “polymorphism” refers to the presence of two or more variant forms of a specific DNA sequence that can occur among different individuals or populations. The most common type of polymorphism involves variation at a single nucleotide, called single-nucleotide polymorphism (SNP). Standard methods of long-read DNA sequencing such as with Oxford Nanopore or PacBio systems (Feng et al., 2021, Nature Communications 12: 3032) can be used to identify SNP and other polymorphic variants that are present specifically on WT/full-length TTN allele but not TTN mutated allele.

As disclosed herein, delivery vehicles for the therapeutic embodiments of the invention capable of affecting, in particular increasing, gene expression in a target tissue in an individual in need thereof, include but are not limited to lipid nanoparticles, including PEGylated embodiments thereof (conjugated with polyethylene glycol) (see, Saupe & Rades, 2006, Nanocarrier Technologies, p. 41; Jenning et al., 2000, Intl. J. Pharmaceut. 199: 167-177; Tumbull et al., Mol. Ther. 24: 66-75; Afzelius et al., 1989, Biochim. Biophys. Acta 979: 231-238), adeno-associated virus (AAV) constructs (Fuentes & Schaffer, 2018, Curr. Opin. Biomed. Engin. 7: 33-41; Xu et al., 2019, Viruses 11: 28; Wong et al., 1986, Clin. Exp. Pharmacol. Physiol. 13: 267-270; Tabebordbar et al., 2021, Cell 184: 4919-4938), lentivirus constructs (Yip, 2020, Biomolecules 10:839; Yudovich et al., 2020, Nat. Sci. Report. 10: 22393; Uchida et al., 202, Cell 21: 121-132; Niwano et al., 2008, Mol. Ther. 16: 1026-1032) adenovirus constructs (Ehrke-Schulz et al., 2017, Nat. Sci. Report. 7: 7113; Boucher et al., 2019, J Control Release 327: 788-800; Raake et al., 2004, J. Am. Coll. Cardiol 44: 1124-1129), modified RNA species (Huang et al., 2015, Molec. Pharmacol. 12: 991-6), and endosomes comprising said constructs (Hundy et al., 2016, Gene Ther. 23: 380-392; Gyorgy & Maguire, 2017, WIREs 10: e1488; Orefice, 2020, Pharmaceutics 2020 12: 705; Sancho-Albero et al., 2020, RSC Adv. 10: 23975; Pofali et al., 2020, Curr. Cancer Drug Targets 20:821-830; Liu et al., 2021, Front. Cell Dev. Biol. doi.org/10.3389/fcell.2021.707607; Metzner & Zaryuba, 2021, Viruses 13: 1238). In particular, lipid nanoparticles can comprise mono-, di-, and triglycerides, fatty acids, steroids, and sterols such as cholesterol phospholipids, sphingosines and sphingomyelin, bile salts such as sodium taurocholate, as well as emulsifiers. See, Shah et al., 2015, Lipid Nanoparticles: Production, Characterization and Stability.

In various aspects, the disclosure provides a pharmaceutical composition comprising a therapeutically effective amount of a compound of the disclosure, and one or more pharmaceutically acceptable diluents, preservatives, solubilizers, emulsifiers, adjuvants, excipients, or carriers.

In certain aspects, the disclosure provides for a pharmaceutical composition comprising the compounds of the disclosure together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, and the like.

The term “pharmaceutically acceptable vehicle” refers to a diluent, adjuvant, excipient, or carrier with which a compound of the disclosure is administered. The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. An appropriate “effective” amount in any individual case can be determined by one of ordinary skill in the art using routine experimentation.

“Pharmaceutically acceptable carriers” for therapeutic use are well known in the pharmaceutical art, and are described, for example, in Remington's Pharmaceutical Sciences, 18th Edition (Easton, Pennsylvania: Mack Publishing Company, 1990). For example, sterile saline and phosphate-buffered saline at physiological pH can be used. Preservatives, stabilizers, dyes, and even flavoring agents can be provided in the pharmaceutical composition. For example, sodium benzoate, sorbic acid and esters of p-hydroxybenzoic acid can be added as preservatives. Id. at 1449. In addition, antioxidants and suspending agents can be used. Id.

Suitable excipients for non-liquid formulations are also known to those of skill in the art. A thorough discussion of pharmaceutically acceptable excipients and salts is available in Remington's Pharmaceutical Sciences, 18th Edition (Easton, Pennsylvania: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be provided in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

In general, the compositions of the disclosure will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

Thus, the compositions of the disclosure can be administered as pharmaceutical formulations including those suitable for parenteral (including intramuscular, intracardiac, intra-arterial, intrathecal, subcutaneous and intravenous) administration or in a form suitable for administration by inhalation or insufflation. The preferred manner of administration is intravenous, intra-arterial or intracardiac using a dosage regimen which can be adjusted according to the degree of affliction.

In yet another embodiment is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and thiolated polymers (carboxymethyl cellulose-cysteine, polycarbophil-cysteine, chitosan-thiobutylamidine, chitosan-thioglycolic acid, chitosan-glutathione conjugates).

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing, or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution, and isotonic sodium chloride solution. In addition, sterile, fixed oils, fatty esters, or polyols are conventionally employed as solvents or suspending media. In addition, parenteral administration can involve the use of a slow release or sustained release system such that a constant level of dosage is maintained.

Parenteral administration includes intraarticular, intravenous, intracardiac, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as a continuous infusion system. A formulation provided by the disclosure can be administered using a syringe, injector, pump, or any other device recognized in the art for parenteral administration.

Preferably, sterile injectable suspensions are formulated according to techniques known in the art using suitable carriers, dispersing, or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution, and isotonic sodium chloride solution. In addition, sterile, fixed oils, fatty esters, or polyols are conventionally employed as solvents or suspending media. In addition, parenteral administration can involve the use of a slow release or sustained release system such that a constant level of dosage is maintained.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

The pharmaceutical compositions of the disclosure can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

The compositions of the disclosure can be formulated for aerosol administration, particularly to the respiratory tract and including intranasal administration. The compound will generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dose of drug can be controlled by a metered valve. Alternatively the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of e.g., gelatin or blister packs from which the powder can be administered by means of an inhaler.

A pharmaceutically or therapeutically effective amount of the composition is delivered to the subject. The precise effective amount can vary from subject to subject and depends upon the species, age, the subject's size and health, the nature and extent of the condition being treated, recommendations of the treating physician, and the therapeutics or combination of therapeutics selected for administration. Thus, the effective amount for a given situation can be determined by routine experimentation. For AAV, generally a therapeutic amount will be in the range of 1×10¹³ vg/kg (viral genomes per kilogram of patient) to 5×10¹⁴ vg/kg. For lipid nanoparticles, this could be 1 or more mg/kg (mg nanoparticle over kilogram of patient) (estimated dosages from Manso et al., 2020, Sci Trans/Med 12: eaax1744 for targeting cardiac LAMP2 using AAV in mice, and Rothgangl et al., 2021, Nat. Biotechnol. 39: 949-957 as reference for targeting liver for PCKS9 in non-human primates). The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

Pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Definitions

The following terms and expressions used herein have the indicated meanings.

“Pharmaceutically acceptable salt” refers to both acid and base addition salts.

“Therapeutically effective amount” refers to that amount of a compound which, when administered to a subject, is sufficient to effect treatment for a disease or disorder described herein. The amount of a compound which constitutes a “therapeutically effective amount” will vary depending on the compound, the disorder and its severity, and the age of the subject to be treated, but can be determined routinely by one of ordinary skill in the art.

“Modulating” or “modulate” refers to the treating, prevention, suppression, enhancement or induction of a function, condition, or disorder. For example, it is believed that the compounds of the present disclosure can modulate atherosclerosis by stimulating the removal of cholesterol from atherosclerotic lesions in a human.

As used herein, the term “ameliorating” as used with regard to the effect of the methods and pharmaceutical compositions provided herein will be understood by the skilled artisan to mean any positive clinical effect on a DCM patient wherein the disease-related symptoms thereof are diminished, alleviated, or remedied.

“Treating” or “treatment” as used herein covers the treatment of a disease or disorder described herein, in a subject, preferably a human, and includes:
i. inhibiting a disease or disorder, i.e., arresting its development;
ii. relieving a disease or disorder, i.e., causing regression of the disorder;
iii. slowing progression of the disorder; and/or
iv. inhibiting, relieving, or slowing progression of one or more symptoms of the disease or disorder.

“Subject” refers to a warm-blooded animal such as a mammal, preferably a human, or a human child, which is afflicted with, or has the potential to be afflicted with one or more diseases and disorders described herein.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### Examples

The examples set forth herein incorporate and rely on certain experimental and preparatory methods and techniques preformed as exemplified herein.

Induced Pluripotent Stem Cell (iPSc) Culture and Cardiomyocyte (CM) Differentiation

All cardiomyocyte (CM) experiments were performed using parental PGP1 GM23338 (obtained from Coriell Institute Biorepository) iPSC line, a commercially available wildtype control line previously used to study CM and sarcomere pathobiology (Hinson et al., 2015, Science 349: 982-986; Hinson et al., 2016, Cell Rep. 17: 3292-3304; Chopra et al., 2018, Dev. Cell. 44: 87-96; Ng et al., 2019, JCI Insight 5; Cohn et al., 2019, Stem Cell Reports 12: 71-83). PGP1 iPSCs, and all CRISPR-engineered derivatives, were seeded onto Matrigel-coated tissue culture plates (Corning 354230) in mTeSR1 (STEMCELL Technologies 85875) containing 10 M ROCK inhibitor Y-27632 (Tocris 1254). mTeSR1 was replenished daily until cells reached 80-90% confluency, at which point cells were passaged at a 1:6 ratio using Accutase (BD 561527). Directed differentiation of iPSCs into CMs was accomplished through modulation of WNT/Q-catenin signaling. Briefly, differentiation of iPSCs (90-100% confluent) was initiated through WNT activation by inhibiting GSK-3 with 9-12 μM CHIR99021 (Tocris 4423) for 24 hours in RPMI 1640 (Gibco 11875093) containing B27 (minus insulin) supplement (Gibco A1895601), GlutaMAX (Gibco 35050061), and penicillin-streptomycin (Gibco 15140122). On Day 3 of differentiation, cells were treated with 5 μM IWP-4 (Tocris 5214) for 48 hours to inhibit WNT signaling. On Day 9, media was switched to RPMI containing B27 (plus insulin) supplement (Gibco 17504044). On Day 13, metabolic selection via glucose starvation was performed using glucose-free DMEM (Gibco 11966025) containing 4 mM lactate (Sigma 71718) for 24 hours to obtain >95% CMs. Following selection, CMs were trypsinized (Gibco 25200056) and seeded onto fibronectin-coated tissue culture plates (Gibco 33016015) containing RPMI-B27 supplemented with 2% FBS (GeminiBio 100-106). RPMI-B27 was replenished every other day until analysis (Day 22-35, unless noted otherwise).

### CRISPR Experiments

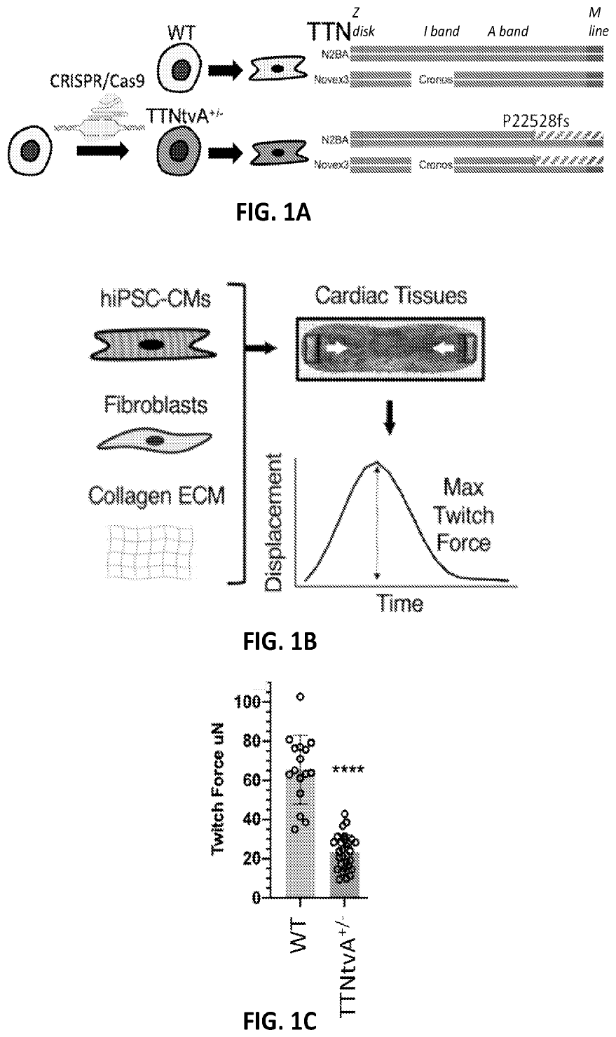

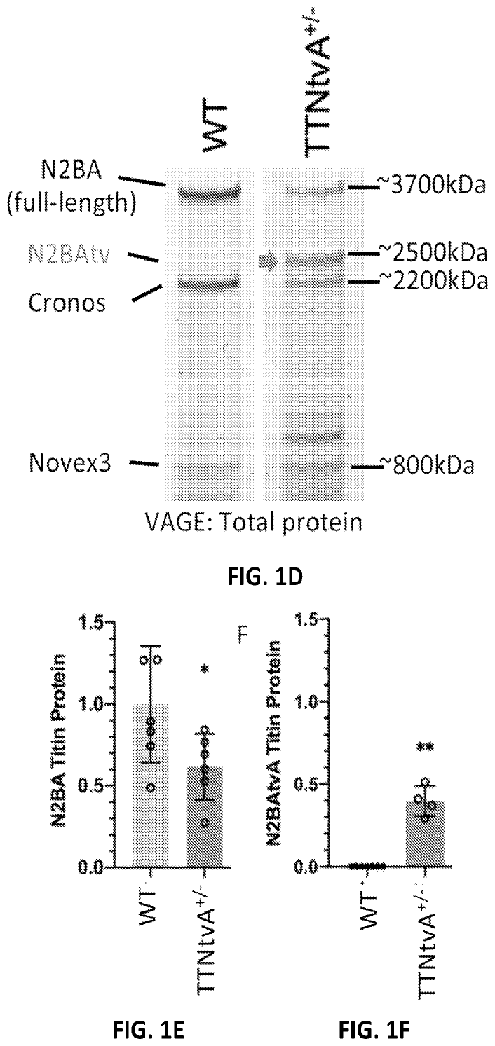

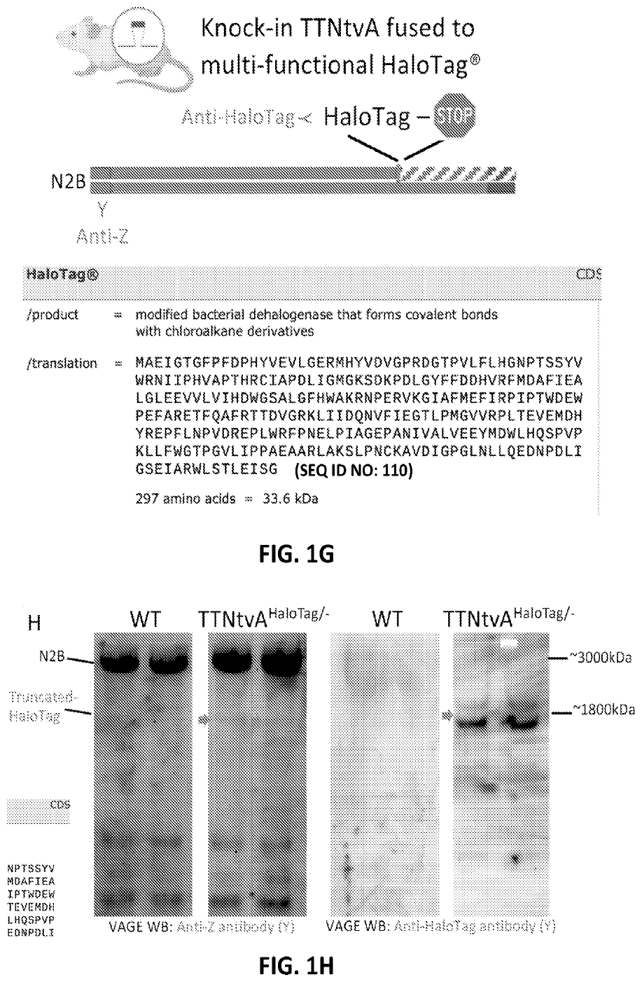

To generate the TNNT2-dCas9-VPR iPSc line set forth hereinbelow, a dCas9-VPR open reading frame was PCR amplified from the Lenti EF1a-FLAG-dCas9-VPR vector (Addgene #114195) and cloned into an HR donor plasmid containing TNNT2 homology arms flanking the TNNT2 stop codon and a T2A linker sequence. To generate the TTN-tdTomato line as described herein, the tdTomato-FLAG open reading frame was obtained as a gBlock (IDT) and cloned into a HR donor plasmid containing TTN homology arms flanking the TTN stop codon. All HR vector propagation steps were performed in DH5α. E. coli (NEB C2987). All isogenic modifications of PGP1 iPSCs were performed using CRISPR/Cas9. The CRISPR-engineered TTNtv^{+/-} iPSC line used for electroporation as set forth below was previously published (Romano et al., 2022, Circulation 145: 194-205). For dCas9-VPR and TTN-tdTomato TTNtv^{+/-} iPSC production, 8×10⁶ iPSCs were electroporated with 20ug pCas9-GFP (Addgene 44719), 20ug of an hU6-driven gRNA, and 20ug of the HR donor plasmid harboring either TNNT2-dCas9-VPR or TTN-tdTomato-FLAG (as shown in FIG. 1B and FIG. 1E). The following day, selection was started with either G418 or Zeocin to isolate single iPSC clones, which were then manually picked onto Matrigel-coated 96-well plates. Once confluent, a portion of each clone was split onto a 96-well plate and the remaining cells were taken for Sanger sequencing and genotyping. Genomic DNA was extracted using the prepGem Universal (Zygem 76218) and the region of interest was PCR amplified using Q5 polymerase (NEB M0491S). All PCR products were then screened and confirmed via Sanger sequencing (Eton Bioscience).

For CRISPRa, guide RNAs (gRNAs) were designed to target TTN (based on N2BA Ensembl transcript 00000591111) based on the hg38 assembly TTN sequence from the UCSC Genome Browser. All gRNAs were cloned into lentiGuide-Puro plasmid (Addgene #52963), packaged in 293T cells and tittered prior to lentiviral transduction into CMs at MOI=10 unless otherwise noted.

### Lentivirus Production and CM Transduction

HEK 293T cells (ATCC CRL-3216) were maintained in DMEM (Gibco 11965092) supplemented with 10% FBS (Gemini 100-106), GlutaMAX (Gibco 35050061), 1 mM sodium pyruvate (Gibco 11360070), and penicillin-streptomycin (Gibco 15140122), and passaged using TrypLE (Gibco 12605028). For lentiviral production, 293T cells were grown to ˜90% confluency and then switched into antibiotic-free media and co-transfected with the desired lentiviral transfer plasmid, psPAX2 packing plasmid (Addgene 12260), and pCMV-VSV-G envelope plasmid (Addgene 8454) using Opti-MEM (Gibco 31985062) and polyethylenimine (PEI). Media was replenished the following day and virus-containing media was harvested on days 2, 3, and 4 post-transfection, followed by concentration using PEG-6000. Functional titers were determined by transducing iPScs with a serial dilution of lentivirus, treating with the appropriate antibiotic (1 μg/mL puromycin or 10 μg/mL Blasticidin), and counting the resistant colony-forming units.

For TTN activation, ˜1×10⁶ CMs expressing dCas9-VPR were first plated onto single wells of a 12-well plate pre-coated with fibronectin, and subsequently transduced using RPMI-B27 containing lentivirus at a multiplicity of infection (MOI) of ˜10 unless otherwise stated. The following day, the cells were replenished with RPMI-B27. Analysis of transduced CMs were performed 7-14 days following transduction.

### Vertical Agarose Gel Electrophoresis (VAGE), SDS-PAGE and Immunoblots

For TTN protein expression analysis, a modified urea sample buffer was used to homogenize CMs directly from 12-well culture plates after the cells were washed once in PBS, which contained 8 M urea, 2 M thiourea, 3% SDS, 75 mM DTT, 0.05 M Tris-Cl (pH=6.8), a protease inhibitor cocktail (1 tab/10 mL) (Roche Diagnostics, Germany 11836170001) and a universal nuclease (0.1 uL/1.0 mL) (Pierce 88700). CM lysates were heated for 10 minutes at 60° C. and centrifuged at 14,000 rpm before use or freezing aliquots to limit freeze-thaw cycles. Samples were normalized to a standard curve of a sarcomere control, α-actinin (ab9465), or GAPDH (ab8245). Non-TTN blots were performed using precast Bio-Rad Mini-PROTEAN TGX gels and transferred onto PVDF membranes (Bio-Rad 1704272) using the Bio-Rad Trans-Blot Turbo system. TTN blots were performed using the Hoefer Dual Gel Casting system and a 1% agarose gel (Lonza 50152) containing 30% glycerol, 0.25 M Tris-Base, 1.92 M Glycine, and 0.5% SDS that was poured between glass plates plugged with an acrylamide gel containing 0.00025% APS and 0.00025% TEMED. The Hoefer SE600 Chroma Vertical Electrophoresis Unit and Hoefer PS300B Power Supply Unit were used to supply 15 mA of constant current for 6 hours while in a refrigerated running buffer containing 50 mM Tris-Base, 0.384 M Glycine, and 0.1% SDS (wherein the upper buffer also contained 10 mM 2-mercaptoethanol). The agarose gel was either stained with SYPRO Ruby (Invitrogen S12000) or transferred to 15×15 cm PVDF membrane (0.45 μm pore size), activated in 100% methanol, by using Hoefer TE42 Transfer Electrophoresis Unit and a transfer buffer containing 24 mM Tris-Base, 192 mM Glycine, and 0.1% SDS. Blots were pre-incubated with 5% milk powder or 5% bovine serum albumin in Tris-buffered saline with Tween (TBST) (10 mmol/L Tris-HCl; pH 7.6; 75 mmol/L NaCl; 0.1% Tween) for 1 hour at room temperature, followed by incubation with the primary antibody overnight at 4° C. The following day, blots were washed three times in TBST for 5 minutes, probed for 1 hour at room temperature with horse radish peroxidase (HRP)-linked secondary antibody (Cell Signaling 7076; 7074), and then washed three times in TBST for 10 minutes. Signal detection was performed using ECL substrate (Thermo 34580) and a Bio-Rad ChemiDoc MP imaging system. Blot images were digitally processed and analyzed in either Bio-Rad Image Lab or ImageJ. Primary antibodies used were as follows: 1:500 anti-TTN-Z1Z2 (TTN-1, Myomedix, Neckargemünd, Germany), 1:500 anti-TTN-M (TTN-9, Myomedix), and 1:1000 anti-GAPDH (5174; Cell Signaling). Additional antibodies used include anti-HaloTag® (G9211, Promega), anti-Cas9 (14697; Cell Signaling), anti-MYH6/7 (HPA001239; Sigma), anti-ACTN (ab9465; Abcam), anti-TNNT2 (MA5-12960; Invitrogen), anti-ATF6α (65880; Cell Signaling), anti-phosphorylated (S51) EIFa (9721; Cell Signaling), anti-EIFa (9722; Cell Signaling), and anti-IRE1a (3294; Cell signaling).

### Cardiac Microtissue Contractility Assay

Cardiac microtissues were generated as described in Cohn et al. (2019, Stem Cell Reports 12: 71-83). Briefly, cantilever devices composed of polydimethylsiloxane (PDMS) (Corning Sylgard 184) were molded from SU-8 silicon masters and embedded with fluorescent microbeads (Thermo F8820) for motion tracking. CMs were mixed with normal human cardiac fibroblasts (Lonza) and spun into PDMS devices containing a collagen-based extracellular matrix (ECM). Tissues were maintained in DMEM+10% FBS, which was replenished daily. For reading frame repair studies, lentiviral SpCas9 and guide RNAS (gRNAs) were added to CMs 5-7 days prior to tissue generation. For acquisition of functional data, tissues were field-stimulated at 1 Hz using a C-Pace EP stimulator (IonOptix). Brightfield and fluorescence videos were acquired on an Andor Dragonfly microscopy system equipped with an enclosed live-cell chamber (Okolabs) and a Zyla sCMOS camera in brightfield and 561-RFP laser widefield modes. Displacement of fluorescent microbeads was tracked using the ImageJ ParticleTracker plug-in, and maximum twitch force was calculated using the cantilever spring constant and cantilever displacement values, as described in Cohn et al. All tissue experiments included a relevant TTN control using a NT (non-targeting) guide RNA to normalize for batch variation in absolute force generation.

### Quantitative PCR, RNA Sequencing and Computational Analyses

RNA was isolated from CMs using TRIzol and phenol-chloroform extraction. cDNA was synthesized using Superscript III First-Strand synthesis (Invitrogen 18080-400). Gene-specific PCR primers (primer sequences listed in Table 1 with SEQ ID NOs: 1-6) were designed using IDT's gRNA designer (as provided by IDT at https://www.idtdna.com/site/order/designtool/index/CRISPR_CUSTOM) and transcripts were quantified using Fast SYBR Green (Applied Biosystems 4385612) on a ViiA7 Real-Time PCR system (Applied Biosystems). For RNA sequencing the RNA was isolated using QIAGEN's RNeasy Mini Kit (Cat #74104). RNA sequencing libraries were generated using KAPA mRNA Hyperprep kit (Roche). Illumina NovaSeq Si flowcell sequencing was conducted with a 100-cycle reagent kit. Estimated total single end reads per sample=34.5-43.1 M 100 bp SE reads. Reads were aligned to the reference hg38 human genome using STAR, quantified with HTSeq, and analyzed using DESeq2. Gene Set Enrichment Analysis (GSEA) was utilized to gene ontologies (GO) terms for datasets. GO terms for genes related to cardiac muscle contraction are represented below and in the accompanying drawings.

TABLE 1
List of Primers used herein.

| | | SEQ |
|---|---|---|
| Name | qPCR primers | ID NO |
| TTN A-band Fwd | CCAGCTGAAACAAAGGAAGG | 1 |
| TTN A-band Rev | CAGGGAAACACTGTCTTTGGA | 2 |
| TTN Z-disk Fwd | TGGGTGTCACTTTTCATTGC | 3 |
| TTN Z-disk Rev | ATGCGCTTGCCATCTTTGTA | 4 |
| Novex-III Fwd | AGAAGATGAAGGAACTTACACG | 5 |
| Novex-III Rev | GGATTTGTGGTCTATGTCTTCAG | 6 |

### Fluorescence-Activated Cell Sorting (FACS)

To quantify TTN-tdTomato levels in CMs, FACS was performed using a BD FACSymphony A5 and FACSDiva software. Prior to FACS, CMs were stained with TO-PRO-3 and Hoechst 33342 to gate for viability and single cells, respectively, and then tdTomato signal was determined (5,000-10,000 cells/sample). All TTN-tdTomato experiments were designed and analyzed using a 96-well plate format (˜10-30 k CMs/well) containing promoter activating guide RNAs or NT control gRNAs for analyzing TTN-tdTomato levels.

### Ttn Promoter Luciferase Assay

To develop a reporter assay that can be used to detect, quantitatively, 17N promoter activity, a lentiviral vector was generated. First, the human TTN promoter was PCR-amplified from human CM genomic DNA (heterozygous for rs72647838 SNP; see Table 7 for all rs-designated genetic loci) corresponding to −600 to 0 relative to the TTN N2BA transcriptional start site and cloned by restriction enzyme digestion followed by sticky-end ligation into a lentiviral plasmid upstream of the open reading frame of NanoLuc luciferase (Promega). After sequencing confirmation of plasmid clones harboring either rs72647838 allele A (GGGG) or B (GG- -), lentivirus was packaged, precipitated and titered (Hunter et al., 209, Nat. Protoc. 4: 495-505). To develop allele-specific TTN activation methods, TTNtv^{+/-} CMs that express dCas9-VPR were transduced with lentivirus encoding TTN promoter driven NanoLuc (at a multiplicity of infection (MOI+=3) as well as gRNAs recognizing sequences overlapping rs72647838 SNP but specific to allele A or B. After 7 days, CMs were lysed, and luciferase activity was measured using the Nano-Glo assay (Promega) and a luminescence plate reader (Biotek). For dCasMini-VPR TTN activation studies, lentiviral dCasMini-VPR (Addgene #176269) was packaged, precipitated and titered, and then co-transduced (MOI=5) into CMs along with TTN promoter driven NanoLuc (MOI=3) and a CasMini-compatible gRNA (MOI=5). After 7 days, CMs were lysed, and luciferase activity was measured using the Nano-Glo assay (Promega) and a luminescence plate reader.

### Statistical Analysis

Data were analyzed and graphed using a combination of statistical programs R and GraphPad Prism. All experiments were conducted with three or more biological replicates (n≥3) unless otherwise indicated. Statistical comparisons were conducted using a Student t test or ANOVA with consecutive Dunnett's correction for multiple comparisons. Statistical significance was defined by P≥0.05 (not significant), P<0.05 (*), P≤0.01 (**), P≤0.001 (***), and P≤0.0001 (****), as set forth below and in the relevant drawings.

### EXPERIMENTAL RESULTS

### Example 1: Engineering DCM-Associated TTNtvs into Human Cardiomyocytes and Mouse Models

The most common and pathogenic DCM-associated Titin (encoded by TTN) mutation type is a protein truncating variant (“tv”) that encompasses frame-shifting insertion, deletion, or splice site mutations, or alternatively a premature stop codon mutation arising within a constitutively expressed exon that encodes for peptides localized to the A-band structural domain (“TTNtvA”) of the protein, where Titin protein interacts with sarcomeric myosin (Schafer et al., 2017, Nat. Genet. 49: 46-53). A human cellular assay was developed to model DCM due to TTNtvs (shown in FIG. 1A) using a previously prepared CRISPR-engineered human induced pluripotent stem cell (iPSc) line harboring a heterozygous single nucleotide deletion variant that results in premature TTN truncation within the A-band structural domain (frameshift after proline-22582; exon 276 in ENST00000591111; Romano et al., 2022, Circulation 145: 194-205). Human cardiomyocytes (CMs) differentiated from these iPSCs using small molecule modulators of WNT signaling in established methods (Lian et al., 2012, Proc. Natl. Acad. Sci. USA 109) were studied as set forth herein. CMs differentiated from this DCM iPSC line are denoted as “TTNtvA^{+/-}”.

TTNtvA^{+/-} and control CM models were functionally interrogated in cardiac microtissues (shown in FIG. 1B), as well as molecular characterizations to determine phenotypic abnormalities underlying highly pathogenic TTNtvs that could be used as an assay for therapeutic development. An advanced 3-dimensional biomimetic cardiac microtissue platform (VAGE; Cohn et al., 2019, Stem Cell Reports 12: 71-83) was used in these experiments because it has been shown to better predict in vivo-like cardiac phenotypes of cardiomyopathy mutations relative to other methods (see, Cohn et al., Id.; Hinson et al., 2015, Science 349: 982-986; Pettinato et al., 2020, Circulation, 142: 2262-2275) to functionally interrogate TTNtvA (shown in FIG. 1C). Using cantilever displacement analysis, TTNtvA^{+/-} nucrotissues relative to controls generated >50% reduced twitch force (a measure of contractile function). This microtissue phenotype was concordant with reduced cardiac contractility observed in human hearts with heterozygous TTNtvs as detected using cardiac ultrasound and identified by diminished cardiac ejection fraction (i.e., the proportion of blood expelled with each heartbeat; see, Herman et al., 2012, N. Engl. J. Med. 366: 619-628). To determine the molecular consequences of TTNtvA^{+/-} on TTN protein levels and sizes, a specialized vertical agarose gel electrophoresis method (VAGE) adapted to study large proteins such as TTN was used (see, Warren et al., 2003, Electrophoresis 24: 1695-197). VAGE analysis of protein lysates obtained from TTNtvA^{+/-} CMs compared to controls revealed >40% decline in full-length TTN protein (N2BA and N2B isoforms) levels (shown in FIG. 1D and FIG. 1E), as well as the presence of an abundant truncation TTN protein not observed in control lysates (shown in FIG. 1D and FIG. 1F). TTNtvs localized to other structural domains such as the I-band also resulted in a decline in full-length TTN protein levels, but no observable truncation TTN protein was detected.

To assess the functional relevance of TTNtvA in vivo, CRISPR was used to introduce a TTNtvA into the equivalent exon as P22582fs (shown in FIG. 1G). To provide a method to track and quantify truncation TTN proteins, multi-functional HaloTag® (Los et al., 2008, ACS Chem Biol. 3: 373-382) were also introduced, proximal to the TTNtvA (TTNtvA^{HaloTag/-}). Using anti-HaloTag® antibodies, the truncated protein products were detected in vivo. Using VAGE, TTN protein quantities and truncation products were assessed in wildtype and TTNtvA^{HaloTag/-} mice. An approximately 15% reduction was observed in full-length TTN protein levels (N2B is the predominant full-length TTN protein in the adult mouse hear; see, Cazoria et al., 2000, Circ. Res. 86: 59-67) and faint N2B truncation protein was detected when immunoprobing with Anti-Z TTN antibodies. Immunoblotting with Anti-HaloTag® antibody confirmed the presence of TTN truncation protein species (shown in FIG. 1H), but at levels lower than were observed in TTNtvA^{+/-} human cardiomyocytes (shown in FIG. 1D). These results showed that a physiological consequence of TTNtv diminished contractile function associated with reductions in full-length TTN protein levels and the acquisition of truncated TTN protein. In addition, reductions in full-length TTN protein levels were a shared consequence of TTNtvs localized to both the I-band and A-band.

### Example 2: TTN Transcriptional Activation Restores TTN Levels and Contractile Deficits Due to TTNtvs

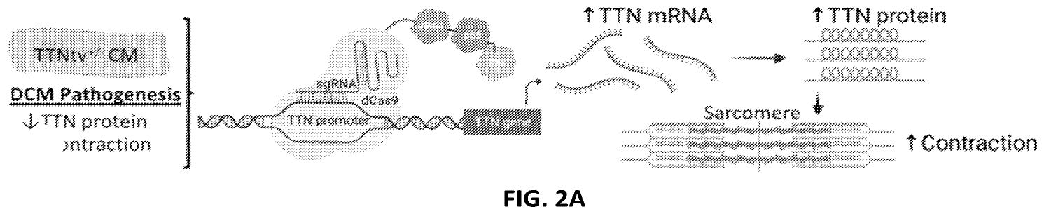

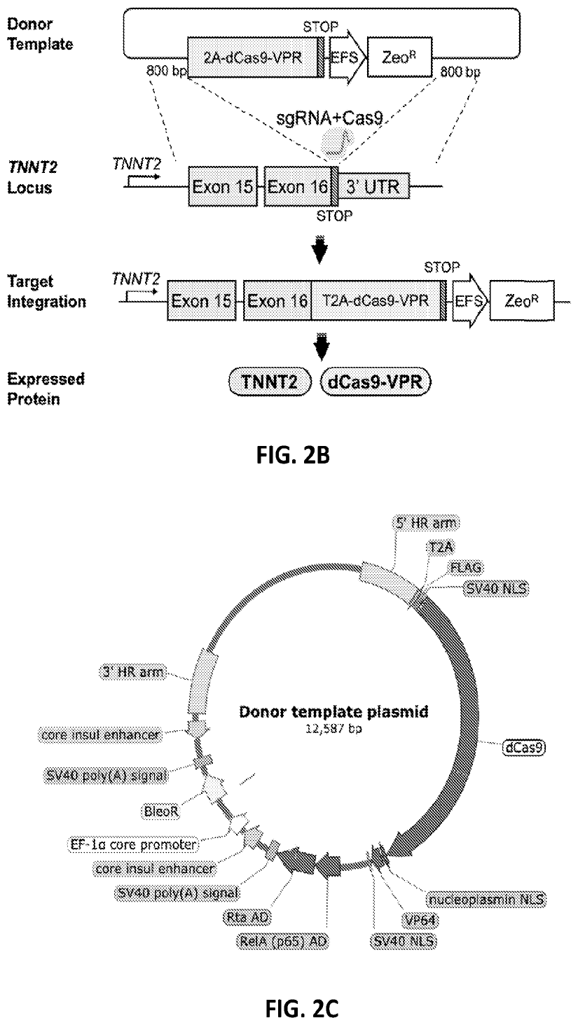

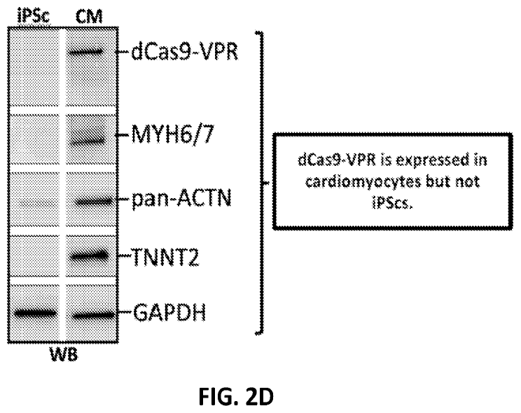

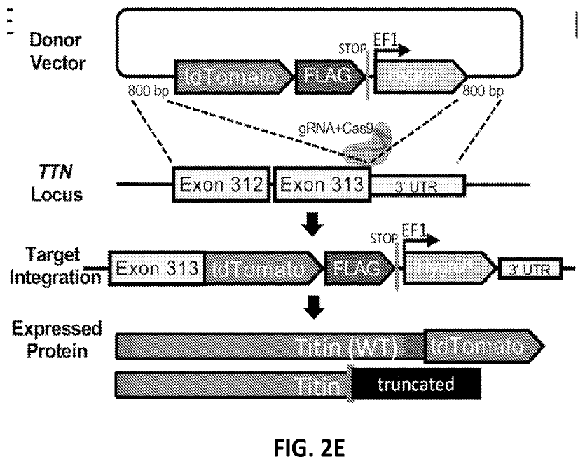

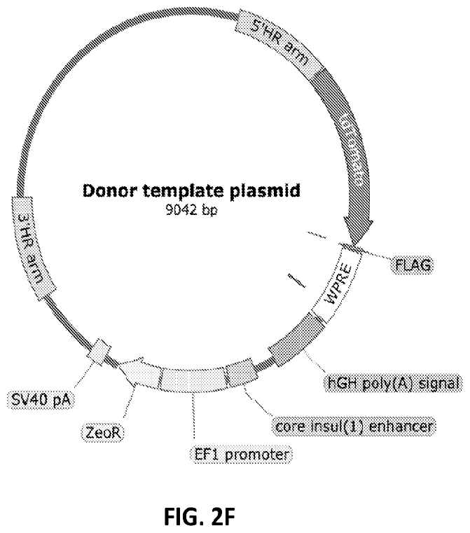

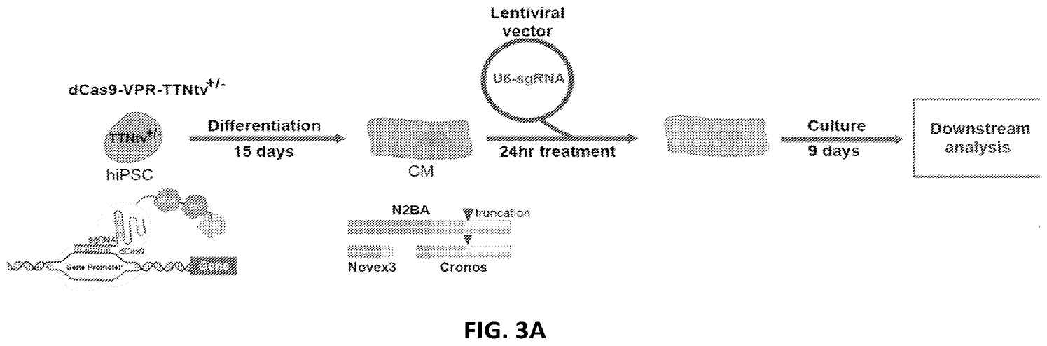

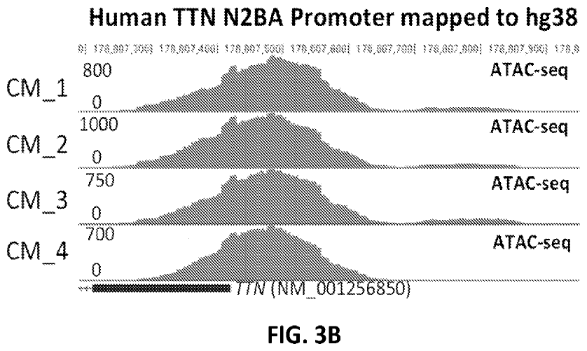

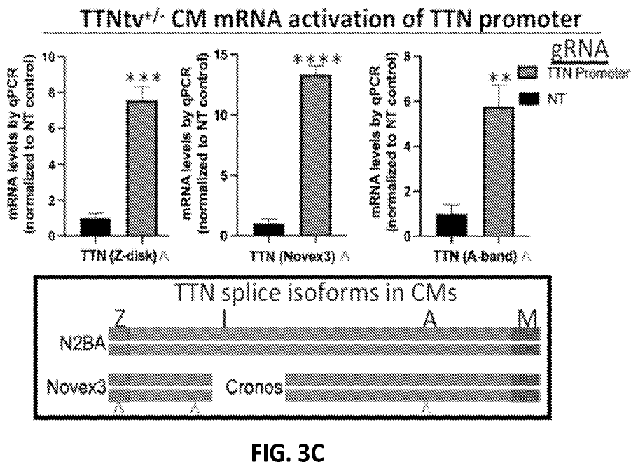

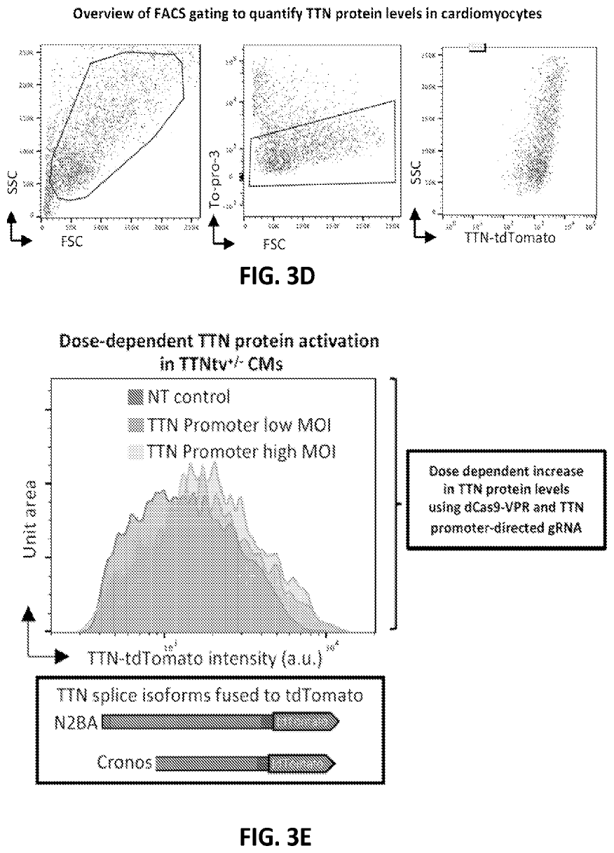

To determine whether the diminished contractility associated with TTNtvs was primarily due to reductions in full-length TTN protein levels (because this was a shared feature of TTNtvs), CRISPR-based transcriptional activation or “CRISPRa” was used. CRISPRa is a recently developed method to increase gene transcript levels through locus-specific recruitment of nuclease-dead Cas9 (“dCas9”) fused to a transcriptional activator domain such as VP64 (see, Chavez et al., 2016, Nature Methods 1: 563-567), illustrated in FIG. 2A. For CRISPRa, dCas9 fused to the tripartite activator complex VP64, p65 and Rta (“dCas9-VPR) was used; VPR had been shown previously to be a strong transcriptional activator in human cell models (Chavez et al., Id.). To generate CRISPRa TTNtvA^{+/-} CMs from hiPSC (FIG. 3A), standard CRISPR/Cas9 was used to facilitate homology-directed repair using a donor template (shown in FIG. 2B and FIG. 2C) containing cardiac troponin T2 (encoded by TNNT2) homology arm sequences to “knock in” dCas9-VPR upstream of the stop codon of TNNT2 using a self-cleaving T2A peptide linker (see, Kim et al., 2011, PLoS One 6: el 8556). After sequence confirmation of TNNT2-T2A-dCas9-VPR^{+/-} iPSc (TTNtvA^{+/-} background) clones, lysates obtained from iPSCs and differentiated CMs were assayed for expression of dCas9-VPR and cardiomyocyte lineage-specific makers including cardiac myosin heavy chains (MYH6 and MYH7) and cardiac troponin T2 (TNNT2) along with GAPDH as a loading control (shown in FIG. 2D). Expression of dCas9-VPR and TNNT2 was observed only in CMs at the expected size, demonstrating both cardiomyocyte-specific expression and efficient T2A peptide cleavage.

A TTN protein reporter was then introduced into the CRISPRa TTNtv^{+/-} CM model to provide a quantitative method to measure TIN levels in individual CMs. Standard CRISPR/Cas9 was used to facilitate homology-directed repair using a donor template (shown in FIG. 2E and FIG. 2F) containing TTN homology arm sequences to introduce tdTomato-FLAG upstream of the stop codon of TTN. After sequence confirmation, a single TTNtvA i-iPSC clone was expanded after visualization of TTN-tdTomato expression upon CM differentiation, thus confirming that tdTomato was fused to the normal (wildtype, functional) TTN allele rather than the TTNtvA allele.

A single guide RNA (gRNA or sgRNA) was then developed, which can be introduced into CMs by lentiviral transduction (lentiGuide-Puro backbone; Addgene #52963; U6 promoter and gRNA scaffold sequences shown in Table 2 of SEQ ID NO: 7) along with dCas9-VPR (sequences shown in Table 3 of SEQ ID NO: 8) to test TTN activation (FIG. 3A). The TTN promoter gRNA was designed to be compatible with SpCas9 (NGG PAM) and programmed to recognize a 20-basepair protospacer sequence (set forth in Table 4) within the N2BA TTN promoter as defined by ATAC-seq peak analysis from CMs (shown in FIG. 3B). The TTN promoter gRNA lentivirus was produced and concentrated, as well as a non-targeting (NT) gRNA lentivirus to serve as control. CMs expressing dCas9-VPR and the TTN promoter gRNA or NT gRNA were transduced and mRNA harvested to quantify TTN transcript levels using quantitative polymerase chain reaction (qPCR). Normalized to NT controls, a 5-13-fold activation of TTN transcript levels with a single TTN promoter gRNA was observed using qPCR (shown in FIG. 3C). TTN isoform activation differences were distinguished based on qPCR amplification of specific transcript sequences using distinct primer pairs overlapping Z-disk TTN sequences that would amplify N2BA and Novex3 isoforms, overlapping Novex3 TTN sequences, and A-band TTN sequences that would amplify N2BA and Cronos isoforms (shown in FIG. 3C). All TTN isoforms were activated, and Novex3 was the most strongly activated. As TTN transcript levels do not always reflect protein levels, the same treatments were performed in TTN-tdTomato^{+/-} CMs expressing dCas9-VPR and TTN protein levels quantified using fluorescence-activated cell sorting (FACS) to measure tdTomato intensity in individual TTNtv^{+/-} CMs. An optimized FACS strategy based on gating for exclusion of non-cellular debris (FSC^{high} and SSC^{high}) was developed and included viable CMs (To-pro-3^{low} and TTN-tdTomato⁺) (shown in FIG. 3D). Using histogram analysis, TTN protein levels in viable TTNtv^{+/-} CMs were observed that could be activated in a dose-dependent pattern as demonstrated by rightward shift of TTN-tdTomato intensity compared to NT controls (FIG. 3E). Taken together, these results showed that CRISPRa using a single gRNA directed to the TTNN2BA promoter can increase TTN transcripts and protein levels using dCas9-VPR. Table 2. U6 promoter and gRNA scaffold sequence utilized herein.

TABLE 2
U6 promoter and gRNA scaffold sequence utilized herein.
U6-dCas9-VPR gRNA (20 mer recognition sequence N20)

gagggcctatttcccatgattccttcatatttgcatatacgatacaaggctgttagagagataattagaattaatttgactgtaaacacaa
agatattagtacaaaatacgtgacgtagaaagtaataatttcttgggtagtttgcagttttaaaattatgttttaaaatggactatcatatgc
ttaccgtaacttgaaagtatttcgatttcttggctttatatatcttgtggaaaggacgaaaCACCGNNNNNNNNNNNN
NNNNNNNNgttttagagctagaaatagcaagttaaaataaggctagtccgttatcaacttgaaaaagtggcaccgagtcggt
gctttttt (SEQ ID NO: 7)

TABLE 3
Sequence of dCas9-VPR.
dCas9-VPR sequence

ATGGACAAGAAGTACTCCATTGGGCTCGCTATCGGCACAAACAGCGTCGGCTGGGCCGTCATTA
CGGACGAGTACAAGGTGCCGAGCAAAAAATTCAAAGTTCTGGGCAATACCGATCGCCACAGCA
TAAAGAAGAACCTCATTGGCGCCCTCCTGTTCGACTCCGGGGAGACGGCCGAAGCCACGCGGCT
CAAAAGAACAGCACGGCGCAGATATACCCGCAGAAAGAATCGGATCTGCTACCTGCAGGAGAT
CTTTAGTAATGAGATGGCTAAGGTGGATGACTCTTTCTTCCATAGGCTGGAGGAGTCCTTTTTGG
TGGAGGAGGATAAAAAGCACGAGCGCCACCCAATCTTTGGCAATATCGTGGACGAGGTGGCGT
ACCATGAAAAGTACCCAACCATATATCATCTGAGGAAGAAGCTTGTAGACAGTACTGATAAGG
CTGACTTGCGGTTGATCTATCTCGCGCTGGCGCATATGATCAAATTTCGGGGACACTTCCTCATC
GAGGGGGACCTGAACCCAGACAACAGCGATGTCGACAAACTCTTTATCCAACTGGTTCAGACTT
ACAATCAGCTTTTCGAAGAGAACCCGATCAACGCATCCGGAGTTGACGCCAAAGCAATCCTGA
GCGCTAGGCTGTCCAAATCCCGGCGGCTCGAAAACCTCATCGCACAGCTCCCTGGGGAGAAGA
AGAACGGCCTGTTTGGTAATCTTATCGCCCTGTCACTCGGGCTGACCCCCAACTTTAAATCTAAC
TTCGACCTGGCCGAAGATGCCAAGCTTCAACTGAGCAAAGACACCTACGATGATGATCTCGACA
ATCTGCTGGCCCAGATCGGCGACCAGTACGCAGACCTTTTTTTGGCGGCAAAGAACCTGTCAGA
CGCCATTCTGCTGAGTGATATTCTGCGAGTGAACACGGAGATCACCAAAGCTCCGCTGAGCGCT
AGTATGATCAAGCGCTATGATGAGCACCACCAAGACTTGACTTTGCTGAAGGCCCTTGTCAGAC
AGCAACTGCCTGAGAAGTACAAGGAAATTTTCTTCGATCAGTCTAAAAATGGCTACGCCGGATA
CATTGACGGCGGAGCAAGCCAGGAGGAATTTTACAAATTTATTAAGCCCATCTTGGAAAAAATG
GACGGCACCGAGGAGCTGCTGGTAAAGCTTAACAGAGAAGATCTGTTGCGCAAACAGCGCACT
TTCGACAATGGAAGCATCCCCCACCAGATTCACCTGGGCGAACTGCACGCTATCCTCAGGCGGC
AAGAGGATTTCTACCCCTTTTTGAAAGATAACAGGGAAAAGATTGAGAAAATCCTCACATTTCG
GATACCCTACTATGTAGGCCCCCTCGCCCGGGGAAATTCCAGATTCGCGTGGATGACTCGCAAA
TCAGAAGAGACCATCACTCCCTGGAACTTCGAGGAAGTCGTGGATAAGGGGGCCTCTGCCCAGT
CCTTCATCGAAAGGATGACTAACTTTGATAAAAATCTGCCTAACGAAAAGGTGCTTCCTAAACA
CTCTCTGCTGTACGAGTACTTCACAGTTTATAACGAGCTCACCAAGGTCAAATACGTCACAGAA
GGGATGAGAAAGCCAGCATTCCTGTCTGGAGAGCAGAAGAAAGCTATCGTGGACCTCCTCTTCA
AGACGAACCGGAAAGTTACCGTGAAACAGCTCAAAGAAGACTATTTCAAAAAGATTGAATGTT
TCGACTCTGTTGAAATCAGCGGAGTGGAGGATCGCTTCAACGCATCCCTGGGAACGTATCACGA
TCTCCTGAAAATCATTAAAGACAAGGACTTCCTGGACAATGAGGAGAACGAGGACATTCTTGAG
GACATTGTCCTCACCCTTACGTTGTTTGAAGATAGGGAGATGATTGAAGAACGCTTGAAAACTT
ACGCTCATCTCTTCGACGACAAAGTCATGAAACAGCTCAAGAGGCGCCGATATACAGGATGGG
GGCGGCTGTCAAGAAAACTGATCAATGGGATCCGAGACAAGCAGAGTGGAAAGACAATCCTGG
ATTTTCTTAAGTCCGATGGATTTGCCAACCGGAACTTCATGCAGTTGATCCATGATGACTCTCTC
ACCTTTAAGGAGGACATCCAGAAAGCACAAGTTTCTGGCCAGGGGGACAGTCTTCACGAGCAC
ATCGCTAATCTTGCAGGTAGCCCAGCTATCAAAAAGGGAATACTGCAGACCGTTAAGGTCGTGG
ATGAACTCGTCAAAGTAATGGGAAGGCATAAGCCCGAGAATATCGTTATCGAGATGGCCCGAG
AGAACCAAACTACCCAGAAGGGACAGAAGAACAGTAGGGAAAGGATGAAGAGGATTGAAGAG
GGTATAAAAGAACTGGGGTCCCAAATCCTTAAGGAACACCCAGTTGAAAACACCCAGCTTCAG
AATGAGAAGCTCTACCTGTACTACCTGCAGAACGGCAGGGACATGTACGTGGATCAGGAACTG
GACATCAATCGGCTCTCCGACTACGACGTGGCTGCTATCGTGCCCCAGTCTTTTCTCAAAGATGA
TTCTATTGATAATAAAGTGTTGACAAGATCCGATAAAGCTAGAGGGAAGAGTGATAACGTCCCC
TCAGAAGAAGTTGTCAAGAAAATGAAAAATTATTGGCGGCAGCTGCTGAACGCCAAACTGATC
ACACAACGGAAGTTCGATAATCTGACTAAGGCTGAACGAGGTGGCCTGTCTGAGTTGGATAAA
GCCGGCTTCATCAAAAGGCAGCTTGTTGAGACACGCCAGATCACCAAGCACGTGGCCCAAATTC
TCGATTCACGCATGAACACCAAGTACGATGAAAATGACAAACTGATTCGAGAGGTGAAAGTTA
TTACTCTGAAGTCTAAGCTGGTCTCAGATTTCAGAAAGGACTTTCAGTTTTATAAGGTGAGAGA
GATCAACAATTACCACCATGCGCATGATGCCTACCTGAATGCAGTGGTAGGCACTGCACTTATC
AAAAAATATCCCAAGCTTGAATCTGAATTTGTTTACGGAGACTATAAAGTGTACGATGTTAGGA
AAATGATCGCAAAGTCTGAGCAGGAAATAGGCAAGGCCACCGCTAAGTACTTCTTTTACAGCAA
TATTATGAATTTTTTCAAGACCGAGATTACACTGGCCAATGGAGAGATTCGGAAGCGACCACTT
ATCGAAACAAACGGAGAAACAGGAGAAATCGTGTGGGACAAGGGTAGGGATTTCGCGACAGTC
CGGAAGGTCCTGTCCATGCCGCAGGTGAACATCGTTAAAAAGACCGAAGTACAGACCGGAGGC
TTCTCCAAGGAAAGTATCCTCCCGAAAAGGAACAGCGACAAGCTGATCGCACGCAAAAAAGAT
TGGGACCCCAAGAAATACGGCGGATTCGATTCTCCTACAGTCGCTTACAGTGTACTGGTTGTGG
CCAAAGTGGAGAAAGGGAAGTCTAAAAAACTCAAAAGCGTCAAGGAACTGCTGGGCATCACAA
TCATGGAGCGATCAAGCTTCGAAAAAAACCCCATCGACTTTCTCGAGGCGAAAGGATATAAAG
AGGTCAAAAAAGACCTCATCATTAAGCTTCCCAAGTACTCTCTCTTTGAGCTTGAAAACGGCCG
GAAACGAATGCTCGCTAGTGCGGGCGAGCTGCAGAAAGGTAACGAGCTGGCACTGCCCTCTAA
ATACGTTAATTTCTTGTATCTGGCCAGCCACTATGAAAAGCTCAAAGGGTCTCCCGAAGATAAT
GAGCAGAAGCAGCTGTTCGTGGAACAACACAAACACTACCTTGATGAGATCATCGAGCAAATA
AGCGAATTCTCCAAAAGAGTGATCCTCGCCGACGCTAACCTCGATAAGGTGCTTTCTGCTTACA
ATAAGCACAGGGATAAGCCCATCAGGGAGCAGGCAGAAAACATTATCCACTTGTTTACTCTGAC
CAACTTGGGCGCGCCTGCAGCCTTCAAGTACTTCGACACCACCATAGACAGAAAGCGGTACACC
TCTACAAAGGAGGTCCTGGACGCCACACTGATTCATCAGTCAATTACGGGGCTCTATGAAACAA
GAATCGACCTCTCTCAGCTCGGTGGAGACAGCAGGGCTGACCCCAAGAAGAAGAGGAAGGTGG
AGGCCAGCGGTTCCGGACGGGCTGACGCATTGGACGATTTTGATCTGGATATGCTGGGAAGTGA
CGCCCTCGATGATTTTGACCTTGACATGCTTGGTTCGGATGCCCTTGATGACTTTGACCTCGACA
TGCTCGGCAGTGACGCCCTTGATGATTTCGACCTGGACATGCTGATTAACTCTAGAAGTTCCGG
ATCTCCGAAAAAGAAACGCAAAGTTGGTAGCCAGTACCTGCCCGACACCGACGACCGGCACCG
GATCGAGGAAAAGCGGAAGCGGACCTACGAGACATTCAAGAGCATCATGAAGAAGTCCCCCTT
CAGCGGCCCCACCGACCCTAGACCTCCACCTAGAAGAATCGCCGTGCCCAGCAGATCCAGCGCC
AGCGTGCCAAAACCTGCCCCCCAGCCTTACCCCTTCACCAGCAGCCTGAGCACCATCAACTACG
ACGAGTTCCCTACCATGGTGTTCCCCAGCGGCCAGATCTCTCAGGCCTCTGCTCTGGCTCCAGCC
CCTCCTCAGGTGCTGCCTCAGGCTCCTGCTCCTGCACCAGCTCCAGCCATGGTGTCTGCACTGGC
TCAGGCACCAGCACCCGTGCCTGTGCTGGCTCCTGGACCTCCACAGGCTGTGGCTCCACCAGCC
CCTAAACCTACACAGGCCGGCGAGGGCACACTGTCTGAAGCTCTGCTGCAGCTGCAGTTCGACG
ACGAGGATCTGGGAGCCCTGCTGGGAAACAGCACCGATCCTGCCGTGTTCACCGACCTGGCCAG
CGTGGACAACAGCGAGTTCCAGCAGCTGCTGAACCAGGGCATCCCTGTGGCCCCTCACACCACC
GAGCCCATGCTGATGGAATACCCCGAGGCCATCACCCGGCTCGTGACAGGCGCTCAGAGGCCTC
CTGATCCAGCTCCTGCCCCTCTGGGAGCACCAGGCCTGCCTAATGGACTGCTGTCTGGCGACGA
GGACTTCAGCTCTATCGCCGACATGGACTTCTCCGCACTGCTGGGTAGCGGATCGGGATCTCGG
GATTCCAGGGAAGGGATGTTTTTGCCGAAGCCTGAGGCCGGCTCCGCTATTAGTGACGTGTTTG
AGGGCCGCGAGGTGTGCCAGCCAAAACGAATCCGGCCATTTCATCCTCCAGGAAGTCCATGGG
CCAACCGCCCACTCCCCGCCAGCCTCGCACCAACACCAACCGGTCCAGTACATGAGCCAGTCGG
GTCACTGACCCCGGCACCAGTCCCTCAGCCACTGGATCCAGCGCCCGCAGTGACTCCCGAGGCC
AGTCACCTGTTGGAGGATCCCGATGAAGAGACGAGCCAGGCTGTCAAAGCCCTTCGGGAGATG
GCCGATACTGTGATTCCCCAGAAGGAAGAGGCTGCAATCTGTGGCCAAATGGACCTTTCCCATC
CGCCCCCAAGGGGCCATCTGGATGAGCTGACAACCACACTTGAGTCCATGACCGAGGATCTGA
ACCTGGACTCACCCCTGACCCCGGAATTGAACGAGATTCTGGATACCTTCCTGAACGACGAGTG
CCTCTTGCATGCCATGCATATCAGCACAGGACTGTCCATCTTCGACACATCTCTGTTTTGA (SEQ
ID NO: 8)

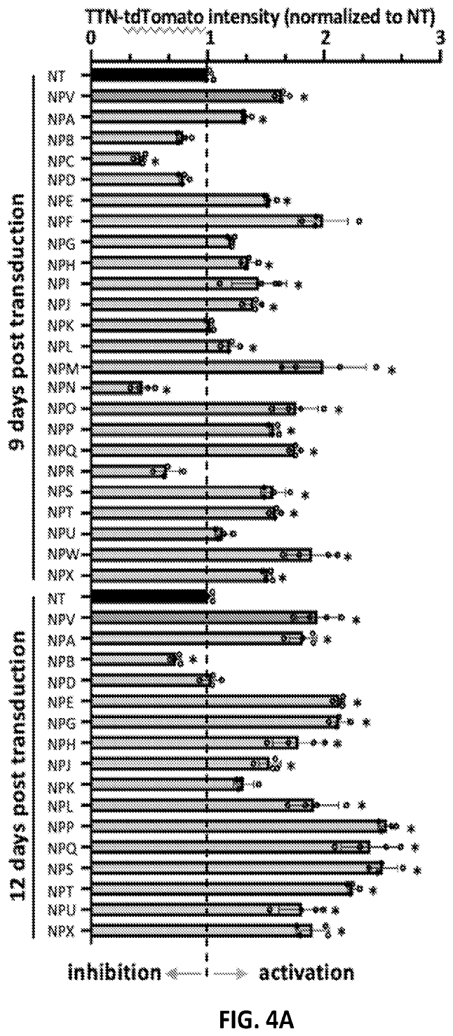

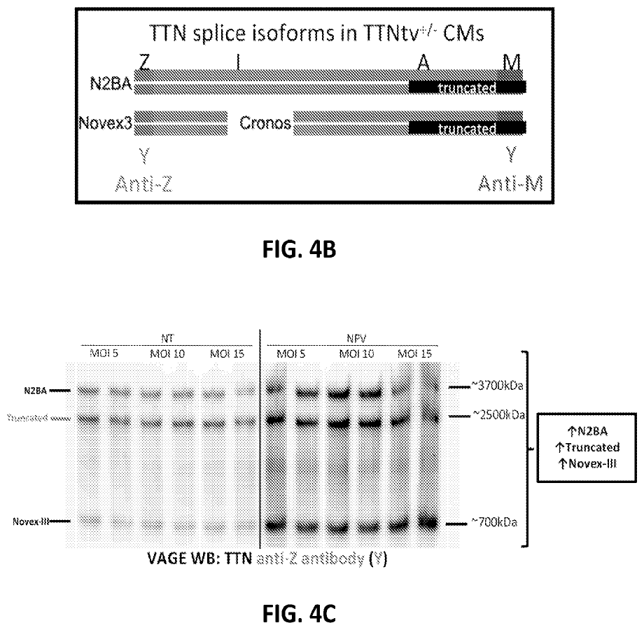

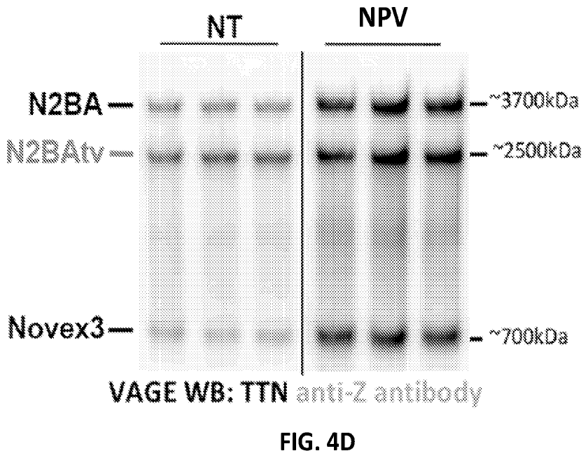

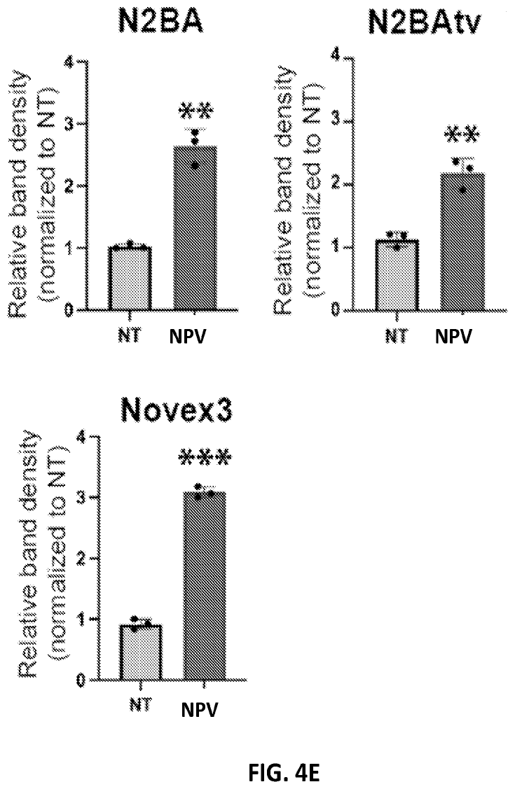

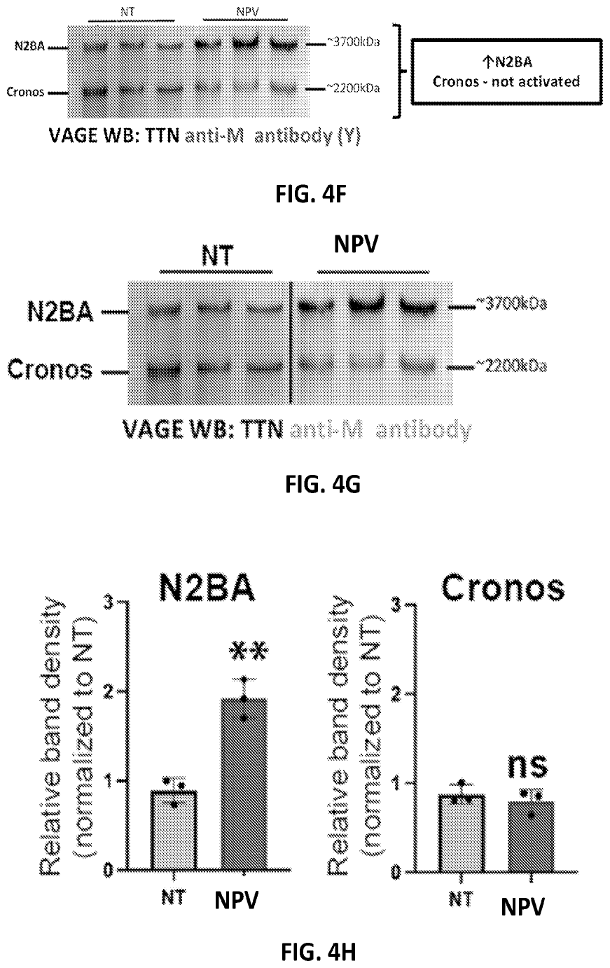

Next, a range of TTN activators (having low->high activity) was identified by screening a larger panel of single gRNAs with the end of the protospacer targeting+97 to −515 relative to the N2BA transcriptional start site. After transducing CMs, TTN activation was tested by FACS at 9 days and 12 days post infection (these results are set forth in Table 4 of SEQ ID NOs: 9-32). Normalized to NT controls, several single gRNAs were observed that activated TTN protein levels more than two-fold (e.g., NPE, NPG, NPP, NPQ, NPS and NPT), several single gRNAs that activated TTN protein levels between one- and two-fold (NPA, NPH, NPJ, NPU and NPX) and a single gRNA that inhibited TTN protein levels (NPB) (shown in FIG. 4A). Time-dependent TTN protein level activation was observed, as TTN-tdTomato levels increased from 9 to 12 days post-transduction (shown in FIG. 4A). Using VAGE to analyze TTN protein isoforms and levels with Anti-Z and Anti-M TTN antibodies after N2BA TTN promoter activation using a strong activator (shown in FIG. 4B), increased levels of N2BA, truncated TTN and Novex3 (FIG. 4C to FIG. 4E); but not Cronos TTN were observed (FIG. 4F to FIG. 4H), and dosage-dependent activation (shown in FIG. 4C).

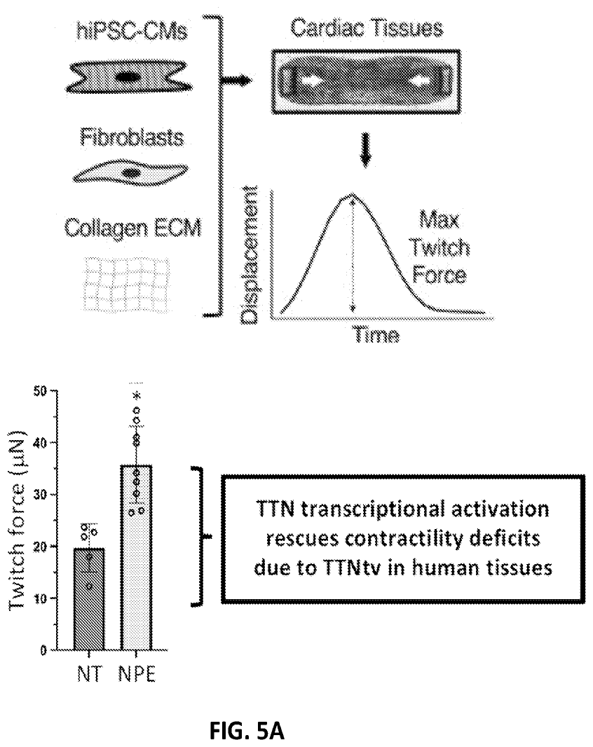

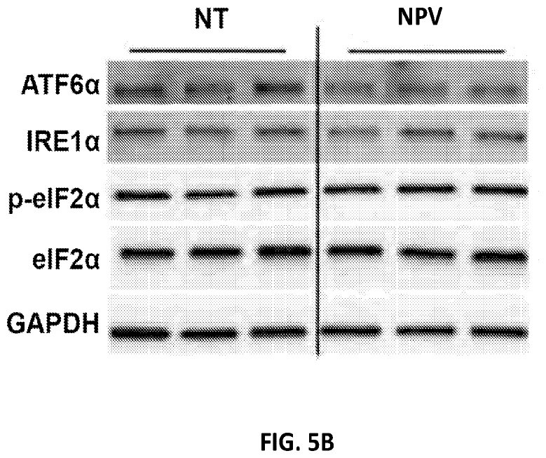

Next, whether TTN activation could rescue TTNtvA-related contractility deficits in cardiac microtissue assays was assessed (see FIG. 5A). Specifically, the NPE gRNA was tested because treatment increased TTN-tdTomato levels in TTNtvA^{+/-} by more than two-fold to levels observed in normal CMs (compare FIG. 1E). TTNtvA^{+/-} cardiac microtissues treated with NPE gRNA demonstrated a greater than 75% increase in twitch force compared to NT controls (shown in FIG. 5A).

Truncated TTN has been previously observed to misfold and aggregate in vivo (Fomin et al., 2021, Sci Transl Med. 13:eabd3079), and increasing TTN levels particularly truncated TTN could be toxic. To screen for toxicity, the activation status of the unfolded protein response (UPR) (Hetz, 2012, Nat Rev Mol Cell Biol. 13:89-102, Glembotski, 2007, Circ Res. 101: 975-84) was tested, which has been previously implicated in cardiomyopathy pathogenesis (Feyen et al., 2021, Circulation 144: 382-392, Wang et al., 2018, Br J Pharmacol. 175: 1293-1304). To test for UPR activation after TTN CRISPRa in dCas9-VPR-TTNtv+/− CMs, protein levels of UPR factors were quantified, including ATF6α, phosphorylated EIF2a and IRE1a using immunoblotting (Borgia et al., 2011, Nature 474: 662-665) lysates (FIG. 5B and FIG. 5C) TTN CRISPRa samples relative to controls showed no change in the levels of these UPR markers. Taken together, these results demonstrate that TTN CRISPRa using dCas9-VPR and single gRNAs directed to the TTNN2BA promoter increase TTN mRNA and protein levels without evidence for UPR activation despite increasing truncated TTN levels.

TABLE 4
List of gRNAs targeted to TTN promoter site.

| | | | G-C | On- | Off- | | SEQ |
|---|---|---|---|---|---|---|---|
| | | | Content | target | target | | ID |
| id | Recognition sequence | Strand | % | Score | Score | hg38 gRNA location | NO |
| TTN NPA | AGTTTTGTTTTTAGGATTAG | (−) | 25 | 63 | 0 | chr2: 178807326-178807345 | 9 |
| TTN NPB | GGGTCTGGCCTGCCCTTATA | (+) | 60 | 17 | 64 | chr2: 178807432-178807451 | 10 |
| TTN NPC | TATAAGGGCAGGCCAGACCC | (−) | 60 | 30 | 52 | chr2: 178807432-178807451 | 11 |
| TTN NPD | GCCCTTATATTCACCAGATC | (+) | 45 | 54 | 82 | chr2: 178807443-178807462 | 12 |
| TTN NPE | TTATATTCACCAGATCTGCT | (+) | 35 | 51 | 37 | chr2: 178807447-178807466 | 13 |
| TTN NPF | CAGCAGATCTGGTGAATATA | (−) | 40 | 25 | 43 | chr2: 178807448-178807467 | 14 |
| TTN NPG | GCTGACAGCCCCACATCCTT | (+) | 60 | 50 | 46 | chr2: 178807464-178807483 | 15 |
| TTN NPH | ACAGCCCCACATCCTTAGGC | (+) | 60 | 34 | 58 | chr2: 178807468-178807487 | 16 |
| TTN NPI | AGGGCCCTGCCTAAGGATGT | (−) | 60 | 25 | 49 | chr2: 178807476-178807495 | 17 |
| TTN NPJ | CTAAGACAGCCAATCACCCA | (−) | 50 | 79 | 60 | chr2: 178807495-178807514 | 18 |
| TTN NPK | GGGTGATTGGCTGTCTTAGG | (+) | 55 | 42 | 61 | chr2: 178807496-178807515 | 19 |
| TTN NPL | GCTGTCTTAGGAAAGCATGA | (+) | 45 | 66 | 7 | chr2: 178807505-178807524 | 20 |
| TTN NPM | CTGTCTTAGGAAAGCATGAT | (+) | 40 | 47 | 0 | chr2: 178807506-178807525 | 21 |
| TTN NPN | TTAGGAAAGCATGATGGGAG | (+) | 45 | 50 | 0 | chr2: 178807511-178807530 | 22 |
| TTN NPO | ATGATGGGAGAGGACCTATT | (+) | 45 | 52 | 56 | chr2: 178807521-178807540 | 23 |
| TTN NPP | ATTTGGTAGTGTCACATACC | (+) | 40 | 56 | 65 | chr2: 178807538-178807557 | 24 |
| TTN NPQ | GGTATGTGACACTACCAAAT | (−) | 40 | 84 | 68 | chr2: 178807538-178807557 | 25 |
| TTN NPR | CTGTCAATCAGAAGAACAGG | (−) | 45 | 55 | 0 | chr2: 178807559-178807578 | 26 |
| TTN NPS | GTTCTTCTGATTGACAGTGC | (+) | 45 | 49 | 46 | chr2: 178807562-178807581 | 27 |
| TTN NPT | GCACTGTCAATCAGAAGAAC | (−) | 45 | 67 | 9 | chr2: 178807562-178807581 | 28 |
| TTN NPU | TGCTAAATTTAGTACCTCAA | (+) | 30 | 65 | 46 | chr2: 178807579-178807598 | 29 |
| TTN NPV | CCTTGGTGAAGTCTCCTTTG | (−) | 50 | 61 | 10 | chr2: 178807596-178807615 | 30 |
| TTN NPW | TAAATACAGTTCTATTTCCT | (−) | 25 | 46 | 0 | chr2: 178807613-178807632 | 31 |
| TTN NPX | AAGTGGATTCTGGAGAATGT | (−) | 40 | 63 | 0 | chr2: 178807938-178807957 | 32 |

### Example 3: RNA-sequencing analysis of TTN transcriptional activation

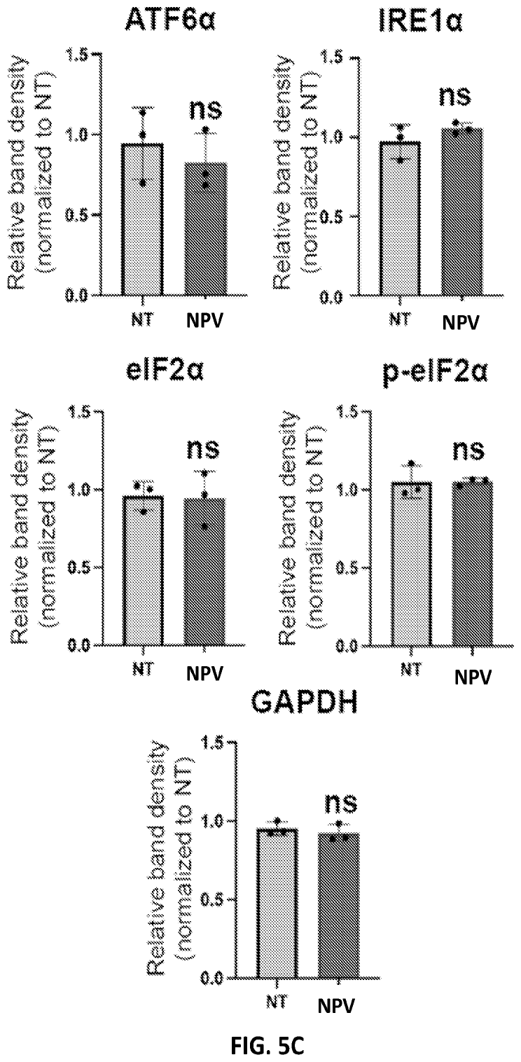

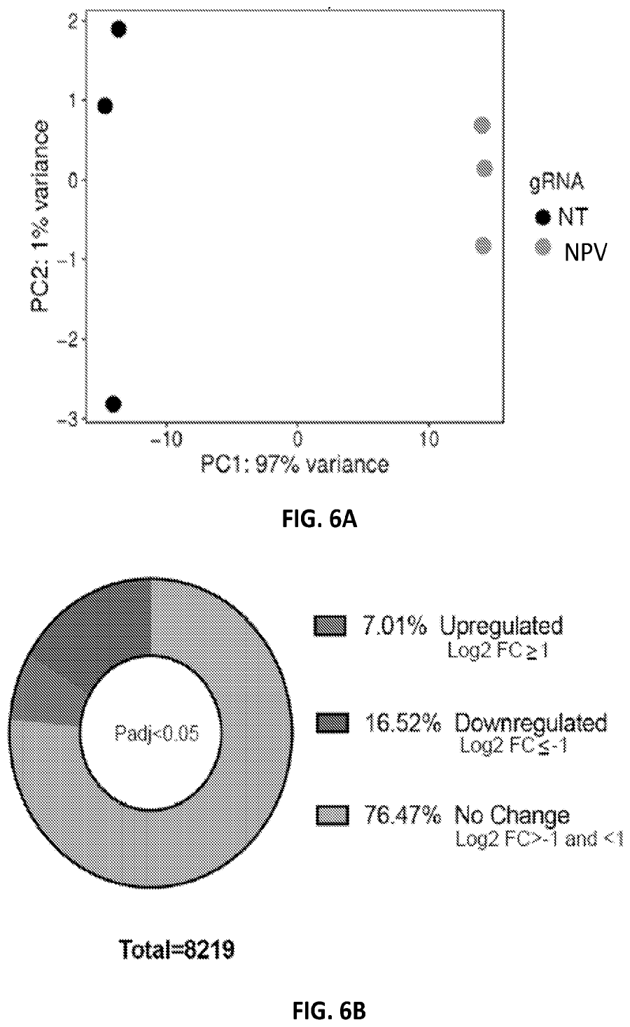

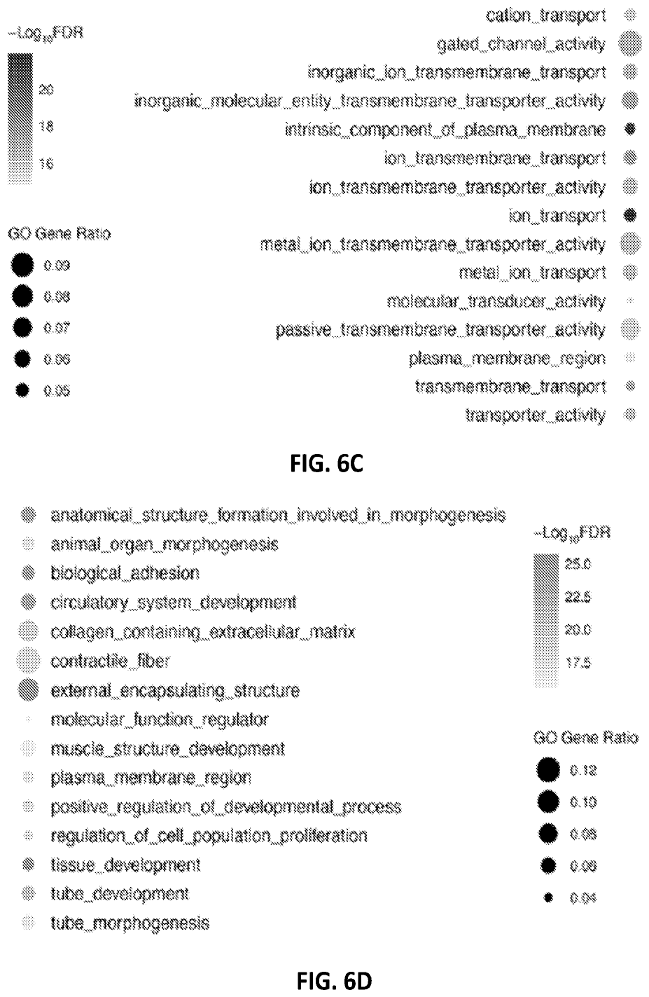

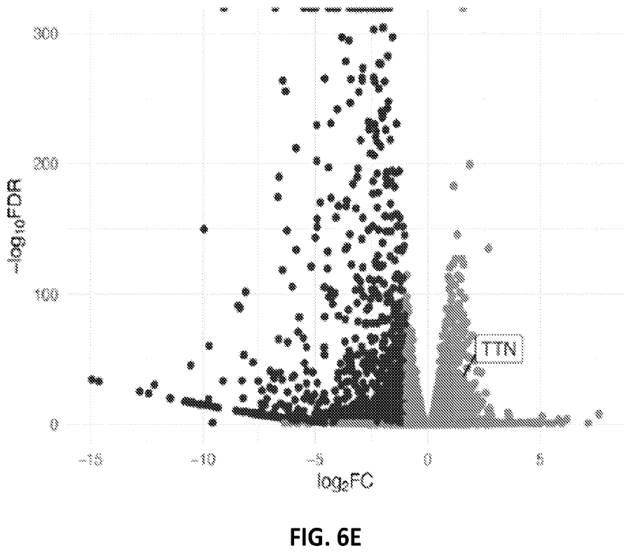

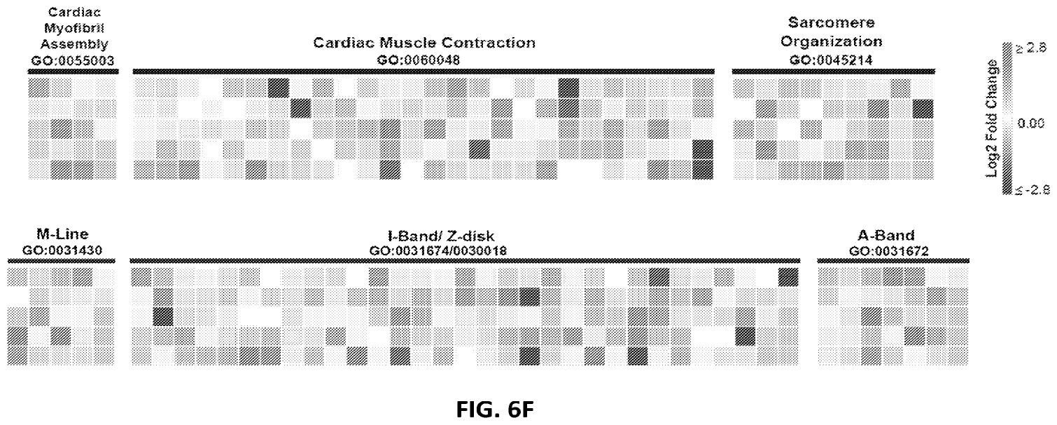

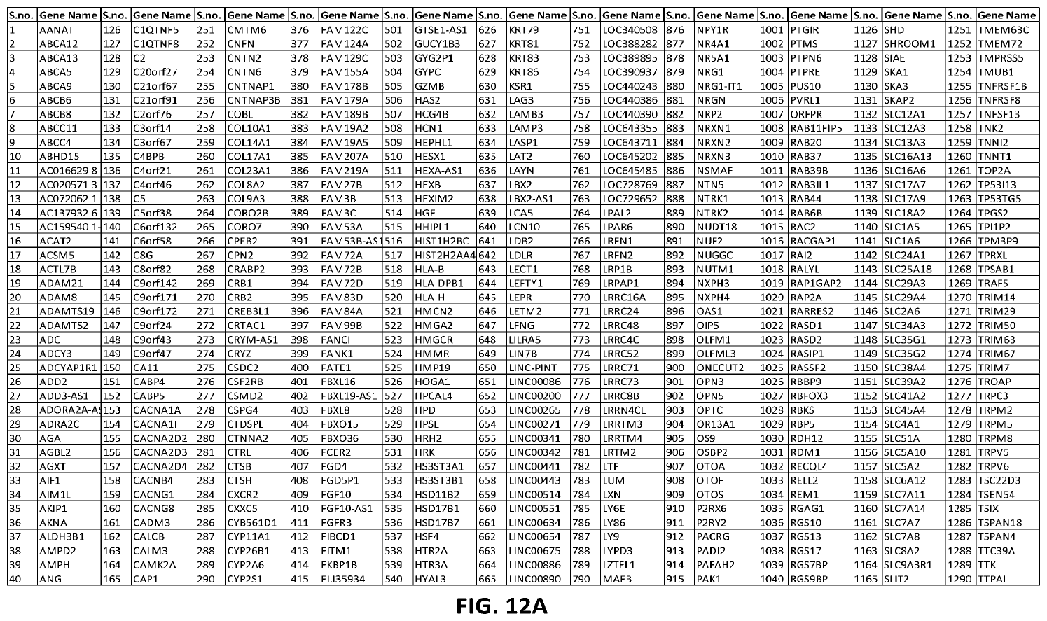

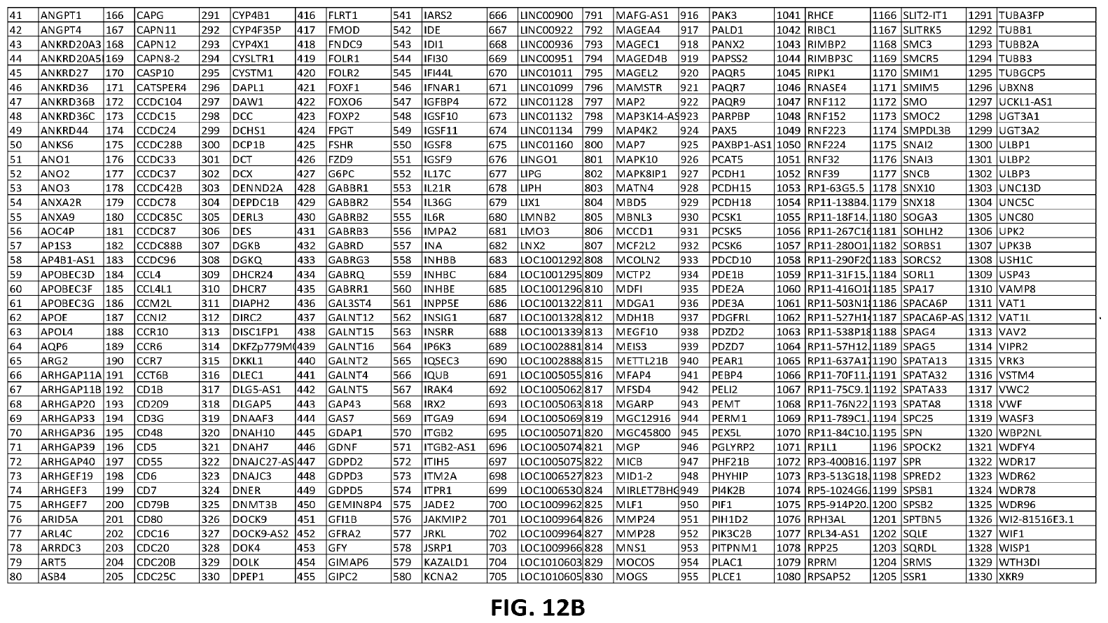

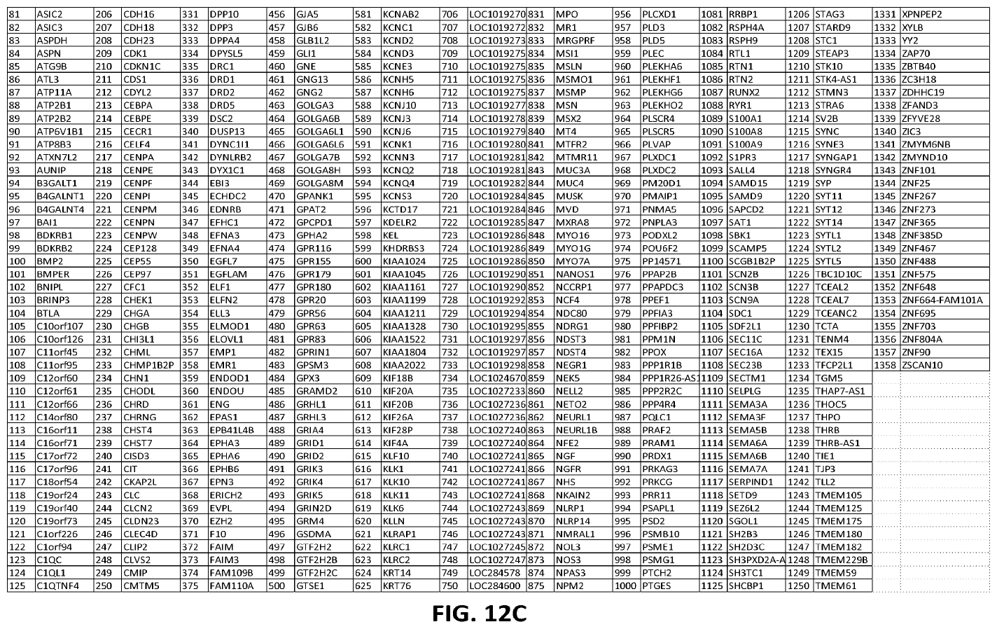

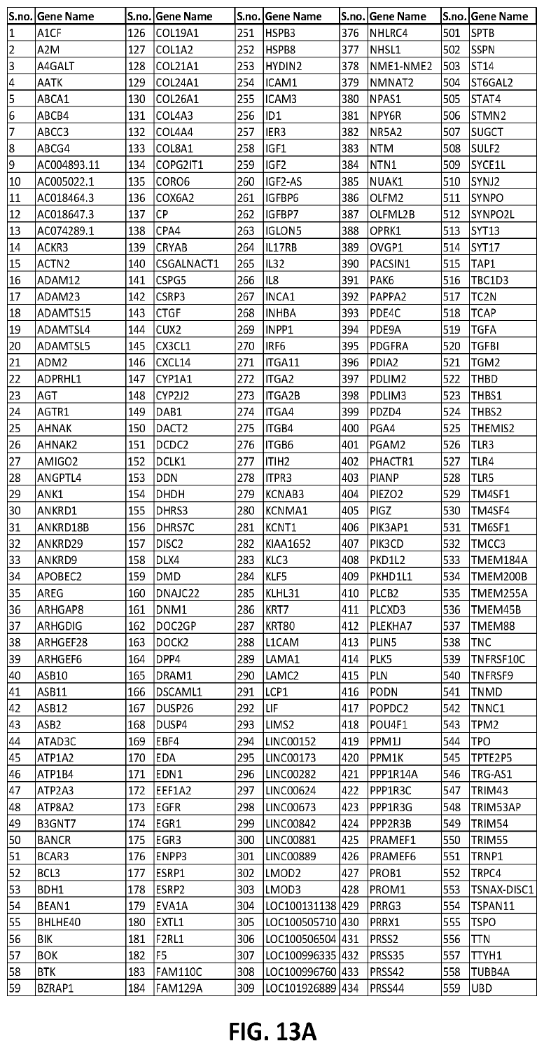

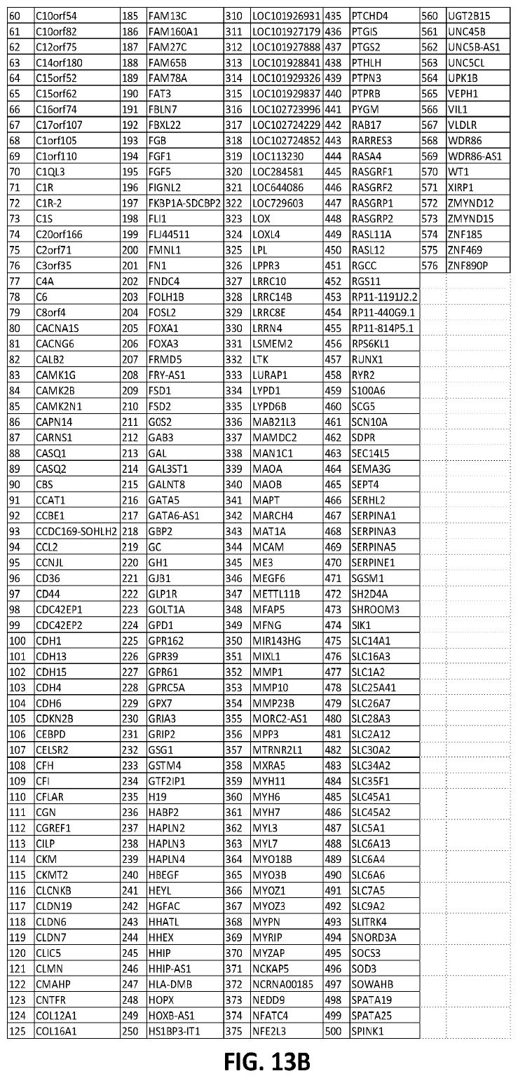

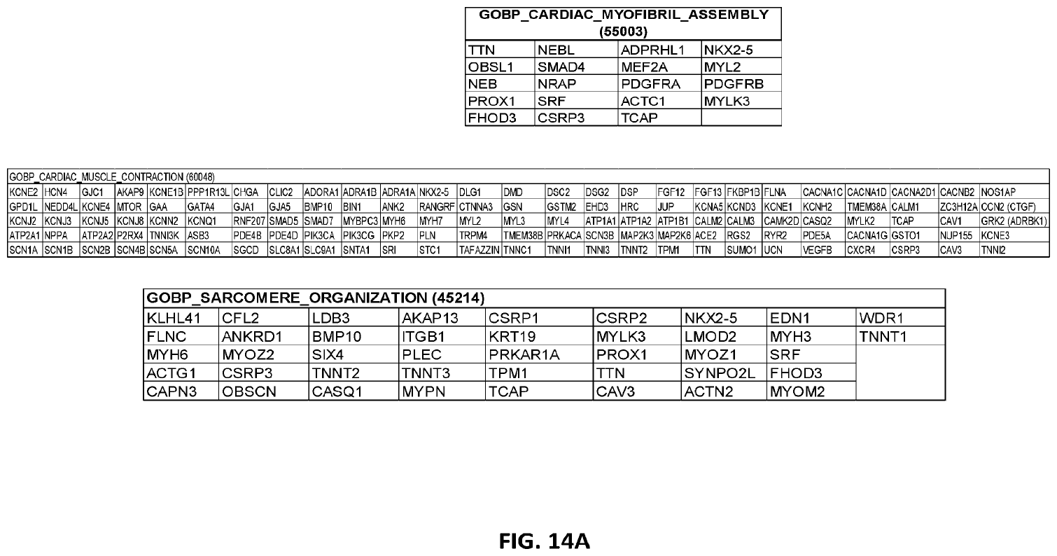

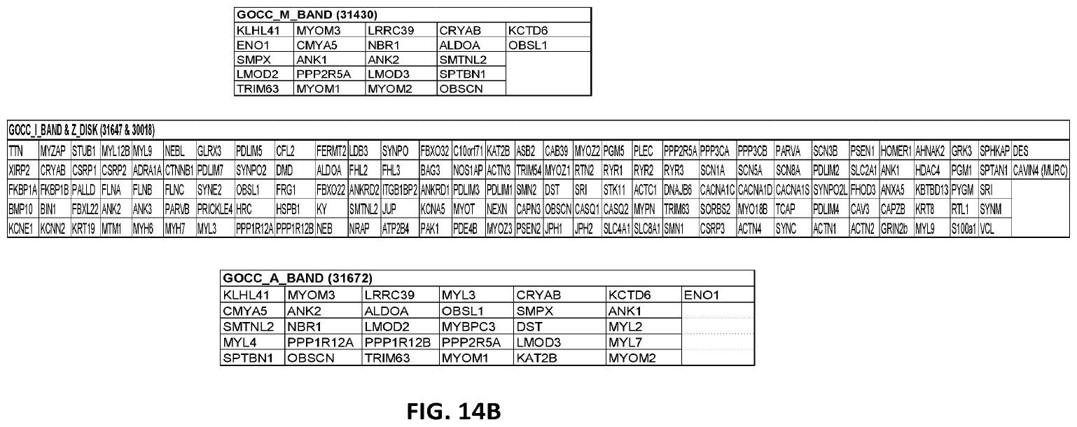

To evaluate the molecular consequences of TTN CRISPRa at the transcriptomic level beyond direct effects on TTN transcripts, RNA sequencing and computational analyses from samples obtained from biological triplicates of NPV and NT control CMs were employed. Principal component analysis of samples demonstrated the distinct separation of NPV from NT biological replicates (FIG. 6A). Differential gene expression (DGE) analysis (cutoffs=P_{adj}<0.01 and log 2FC≥1 or ≤−1) demonstrated 1,357 downregulated (FIG. 12A to FIG. 12C) and 576 upregulated (FIG. 13A and FIG. 13B) transcripts, while most transcripts were unchanged (FIG. 6B). Gene Ontology (GO) term enrichment analysis of the significantly downregulated (FIG. 6C and FIG. 6D) and upregulated (FIG. 6D and FIG. 6E) transcripts revealed changes in biological processes, pathways, and components. GO terms involved in “contractile fiber,” “muscle structure development” and “circulatory system development” were enriched in the upregulated gene sets (FIG. 6E). Plotting fold change heat maps of complete gene sets within GO terms including “cardiac myofibril assembly,” cardiac muscle contraction,” “sarcomere organization,” sarcomere “M-line,” “I-band/Z-disk,” and “A-band” (FIG. 6F, and FIG. 14A to FIG. 14B) illustrated the generalized upregulation of these factors after TTN CRISPRa. GO term enrichment analysis of the downregulated transcripts revealed functions in “ion transport” and “intrinsic component of plasma membrane” (FIG. 6C). To summarize RNA-sequencing results, TTN CRISPRa not only increased TTN transcript levels, but also numerous factors implicated in sarcomere assembly, structure and organization.

### Example 4: TTN Transcriptional Activation Leveraging TTN Regulatory Elements

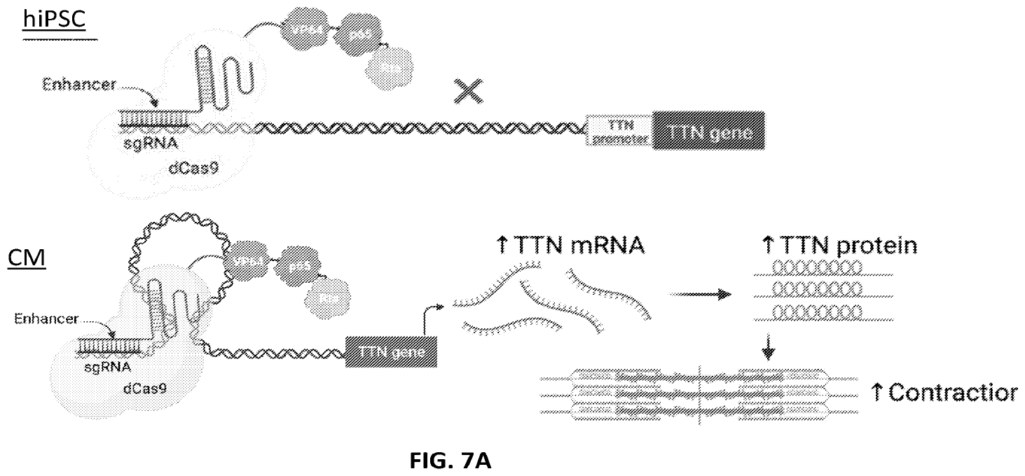

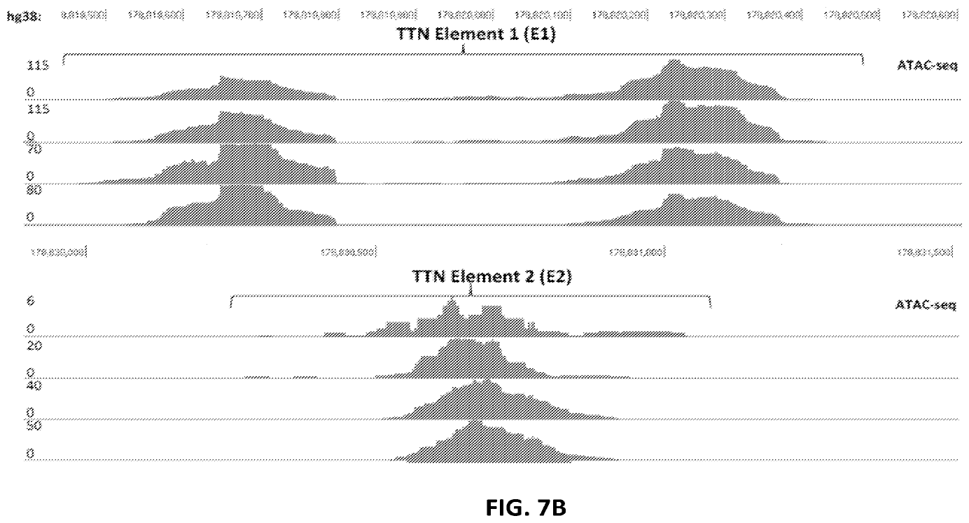

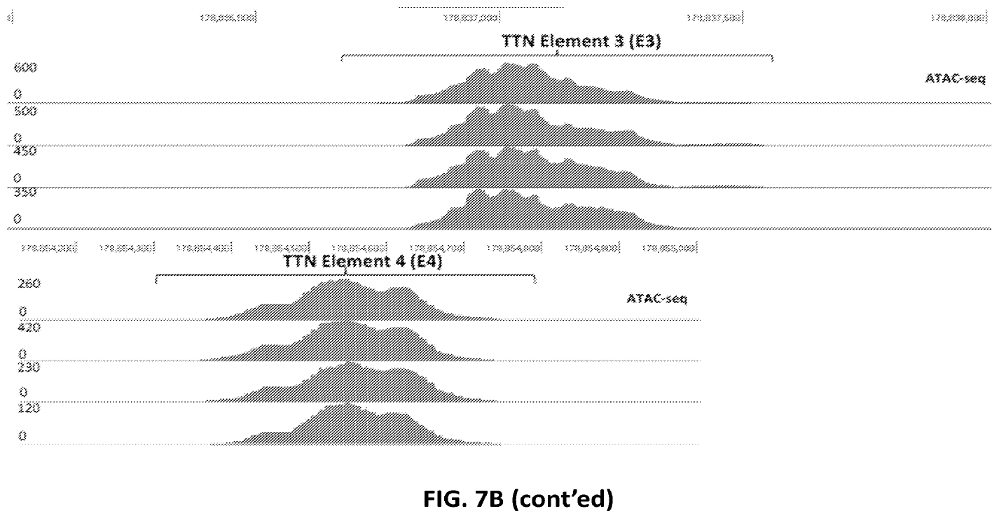

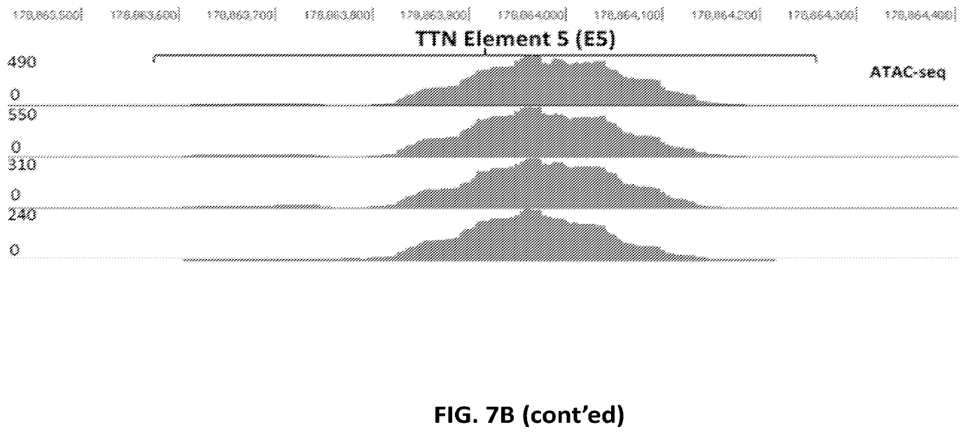

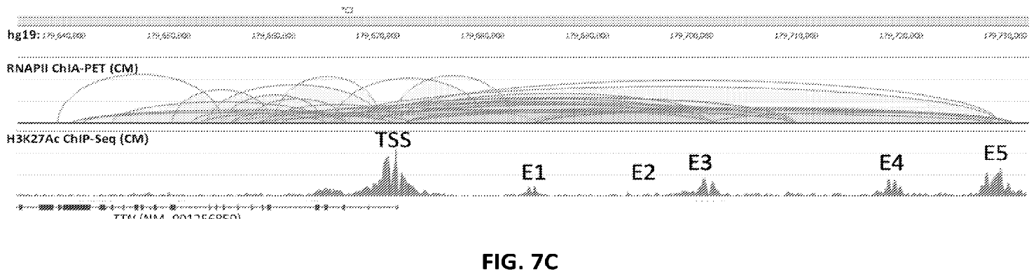

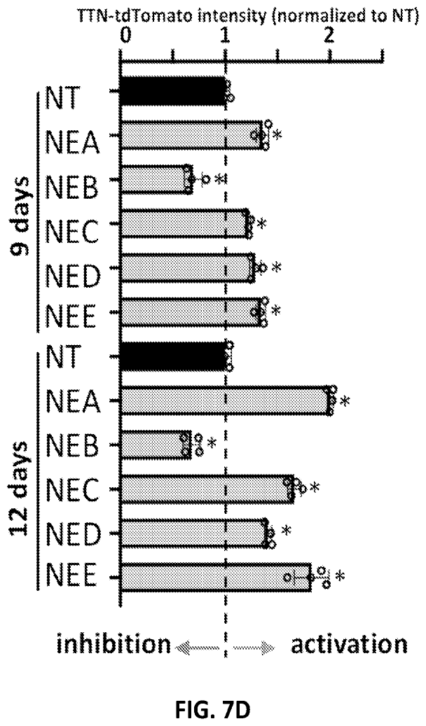

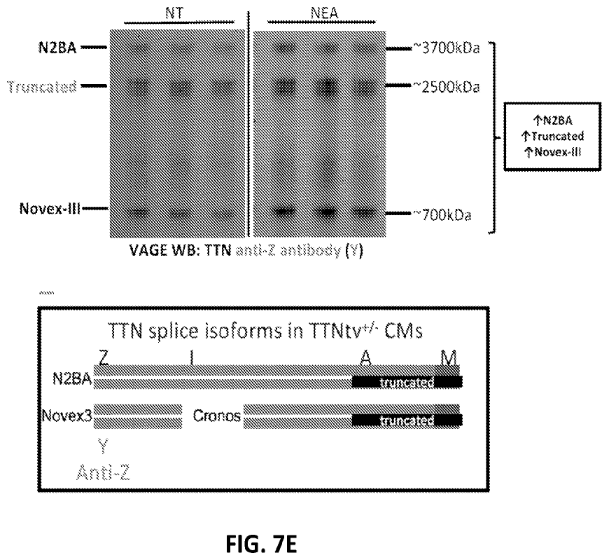

Whether CRISPRa directed to DNA regulatory elements could activate TTN levels in TTNtvA^{+/-} CM models was also tested (shown in FIG. 7A). Cis-acting DNA regulatory elements such as enhancers have been previously demonstrated to make physical contact with gene promoters though 3-dimensional interactions (see, Fullwood et al., 2009, Nature 462: 58-64), such that homing CRISPRa to regulatory elements in physical contact with the TTN promoter could also activate TTN levels and serve as a therapeutic for DCM. These experiments began by examining ATAC-seq data obtained from CM samples for peaks upstream of the TTN TSS. Five TTN elements (E1-E5; shown in FIG. 7B) were focused upon that were supported by peak analysis. To identify TTN elements that contact TTN promoter, chromatin interaction analysis was utilized by paired-end tag sequencing (ChIA-PET) with antibodies recognizing RNA polymerase II (RNAPII), and chromatin immunoprecipitation with DNA sequencing (ChIP-seq) with antibodies recognizing histone H3 lysine 27 acetylation (H3K27ac). ChIA-PET confirmed physical contact between TTN TSS and E1, E3 and E5 (shown in FIG. 7C); while E2 and E4 were only supported by ChIP-seq and ATAC-seq peak analysis (shown in FIG. 7B). Single gRNAs (set forth in Table 5 of SEQ ID NOs: 33-37) were designed that recognize sequences within −250 base pairs of each regulatory element center, and transduced ITNtvA^{+/-} CMs that express dCas9-VPR and TTN-tdTomato therewith. CRISPRa embodiments were identified that targeted E1 (NEA), E3 (NEC), E4 (NED), and E5 (NEE) increased TTN-tdTomato signal in a time-dependent manner (shown in FIG. 7D). CRISPRa E2 (NEB) was identified that resulted in decreased TTN-tdTomato signal suggesting an inhibitory effect. To assess TTN isoforms and levels after TTN element activation, VAGE was used and probed with antibodies to anti-Z TTN. Increased expression of N2BA TTN, truncated TTN and Novex3 isoforms was observed (shown in FIG. 7E) in a similar pattern to TTN promoter activation (compare, FIG. 4C). Taken together, these results supported the conclusion that CRISPRa directed at specific TTN regulatory elements can activate TTN protein resembling TTN promoter activation.

### Example 5: TTN Isoform-Specific Transcriptional Activation Leveraging Internal Promoter

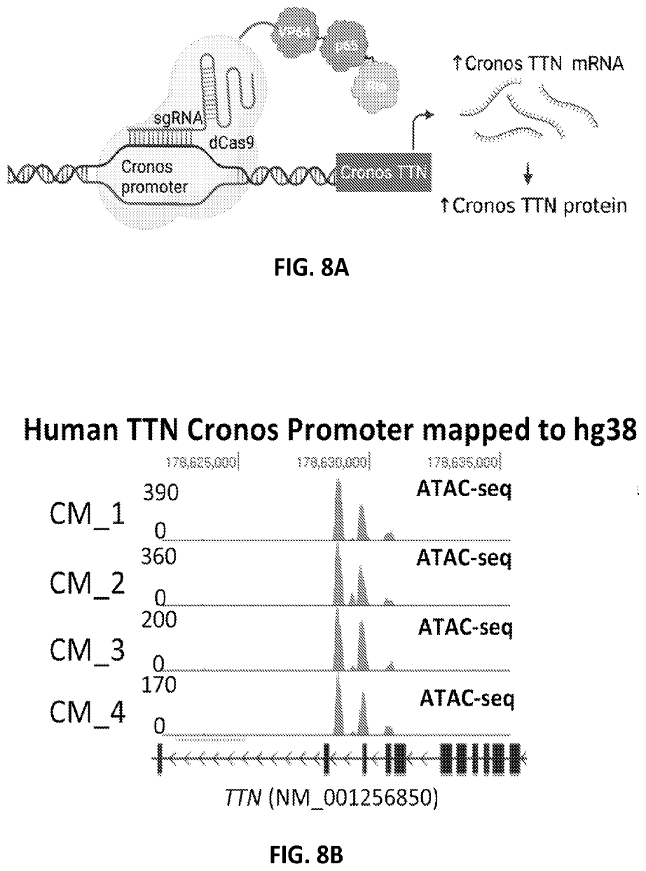

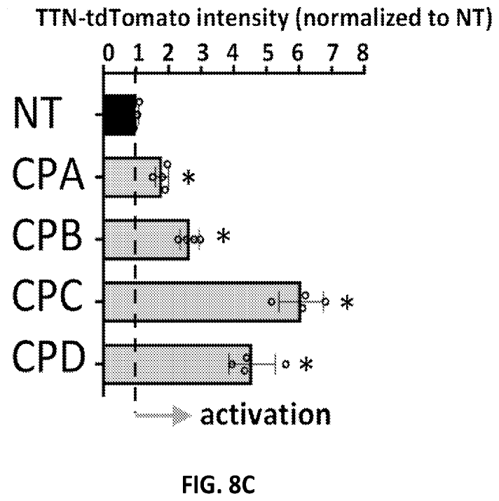

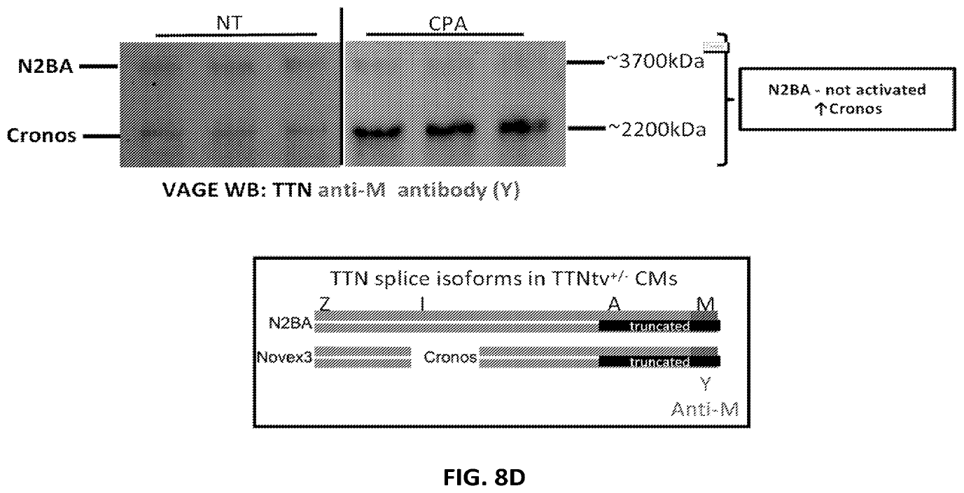

Recently, a TTN internal promoter has been identified that regulates a fetal-enriched TTN isoform called Cronos from the hearts of zebrafish (Zou et al., 2015, Elife 4: e09406) and humans (Zaunbrecker et al., 2019, Circulation 140: 1647-1660). Cronos TTN has been previously demonstrated to regulate myofibril assembly and contractile functions in CMs (see, Xu et al., 2021, Mol. Cell. 81: 4333-4345), but methods to increase Cronos TTN levels (for example, with CRISPRa) have not been previously developed. To test the premise that CRISPRa directed to the Cronos promoter could activate Cronos TTN isoform expression (shown in FIG. 8A) (which could be a therapeutic strategy for DCM), the Cronos TTN promoter was first identified by examining ATAC-seq data obtained from CM samples for peaks near the Cronos TSS (shown in FIG. 8B) and single gRNAs were designed that recognized sequences within −500 base pairs from the Cronos TSS (set forth in Table 6 of SEQ ID NOs: 38-41). TTNtvA^{+/-} CMs that express dCas9-VPR and TTN-tdTomato were transduced with four candidate gRNAs and embodiments of CRISPRa that targeted the Cronos TSS were identified, including CPA-D increased TTN-tdTomato signal (shown in FIG. 8C). Because increased TTN-tdTomato signal could be secondary to increased N2BA and/or Cronos TTN levels, TTN isoforms were examined using VAGE and anti-M TTN antibody using CPA gRNA compared to NT control (shown in FIG. 8D). It was observed in these experiments that CPD activated Cronos TTN, but not N2BA TTN levels. These results demonstrated that methods using CRISPRa and Cronos promoter-directed gRNAs can specifically activate Cronos TTN protein.

TABLE 5
List of gRNAs targeted to TTN enhancer sites in FIG. 7D.
gRNA recognition sequences are shown including distance to N2BA TSS in hg38, strand, G-C 20 content (%), on-target score (IDT),
off-target score (IDT) and hg38 gRNA location.

| | | 5′ boundary of | 3′ boundary of | | G-C | On- | Off- | | |
|---|---|---|---|---|---|---|---|---|---|
| | | TTN regulatory | TTN regulatory | | Content | target | target | | |
| id | Recognition sequence | element (hg38) | element (hg38) | Strand | % | Score | Score | hg38 gRNA location | SEQ ID NO |
| TTN NEA | TTGATACAAGGTTGACATTT | 178819400 | 178820500 | (−) | 30 | 74 | 0 | chr2: 178819760-178819779 | 33 |
| TTN NEB | TGCTGAGACACAACTTCCCC | 178830250 | 178831100 | (−) | 55 | 72 | 18 | chr2: 178830773-178830792 | 34 |
| TTN NEC | ACCAGGCACCTAATTTCTCC | 178836500 | 178837700 | (−) | 50 | 59 | 30 | chr2: 178836983-178837002 | 35 |
| TTN NED | ACATATGATGTATAAGTTTG | 178854385 | 178854659 | (−) | 25 | 62 | 0 | chr2: 178854659-178854678 | 36 |
| TTN NEE | GCTGTGACATTTATGAAGCT | 178863853 | 178864038 | (−) | 40 | 61 | 0 | chr2: 178864038-178864057 | 37 |

TABLE 6
List of gRNAs targeted to TTN promoter sites in FIG. 8C.

| | | | G-C | On- | Off- | | SEQ |
|---|---|---|---|---|---|---|---|
| | | | Content | target | target | | ID |
| id | Recognition sequence | Strand | % | Score | Score | hg38 gRNA location | NO |
| TTN CPA | CAAGTAGTTGAGATAACTTC | (−) | 35 | 92 | 43 | chr2: 178628833-178628852 | 38 |
| TTN CPB | TGCGATATCTGGAATGCAAG | (−) | 45 | 56 | 66 | chr2: 178628749-178628768 | 39 |
| TTN CPC | GGAAGGTGCAAAGTGTTTGA | (−) | 45 | 79 | 13 | chr2: 178629808-178629827 | 40 |
| TTN CPD | CATTTTAGATTCAAGAAAGA | (−) | 25 | 66 | 0 | chr2: 178629856-178629875 | 41 |

### Example 6: TTN Allele-Specific Transcriptional Activation Leveraging Common Genetic Variation

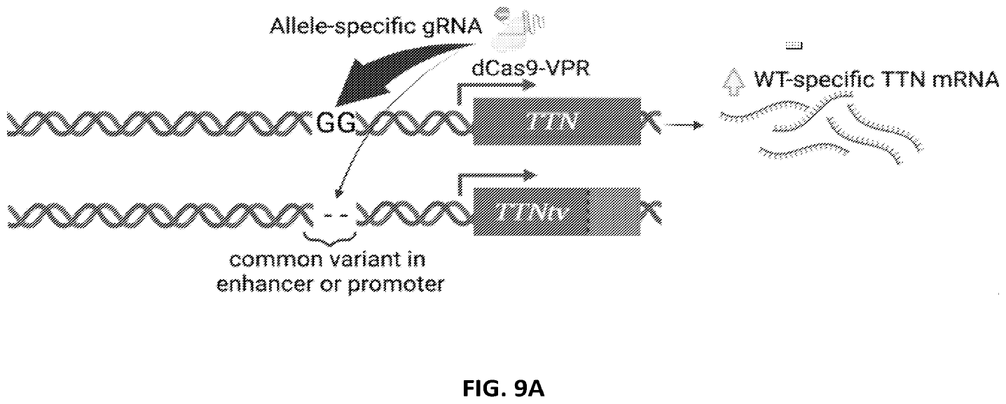

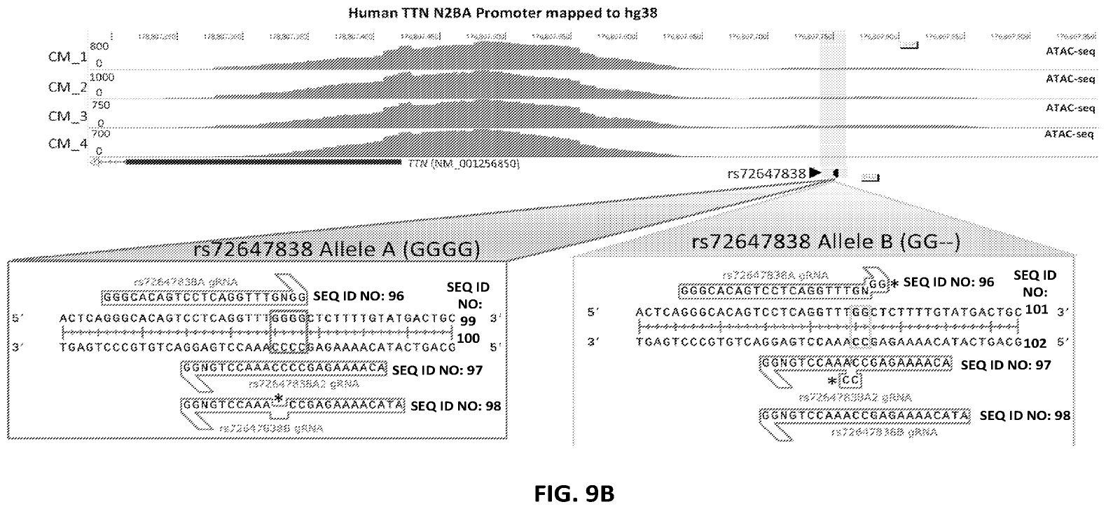

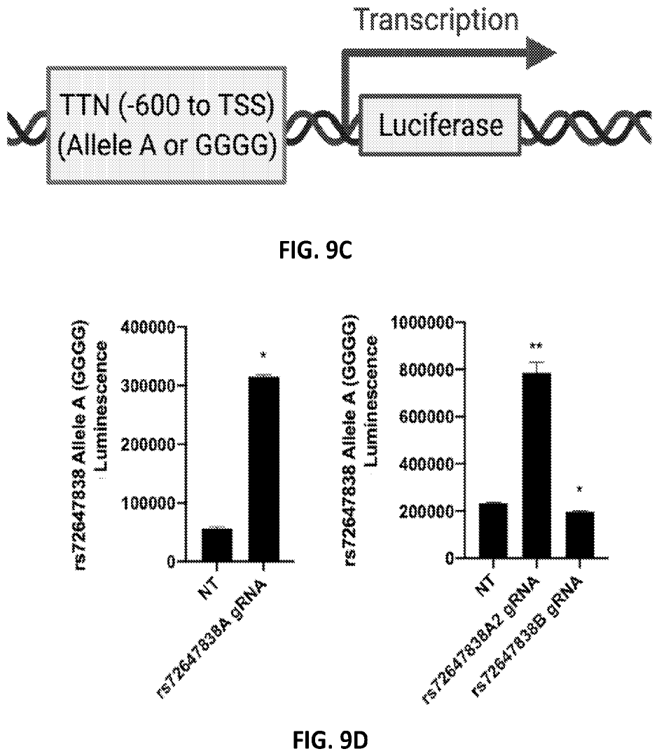

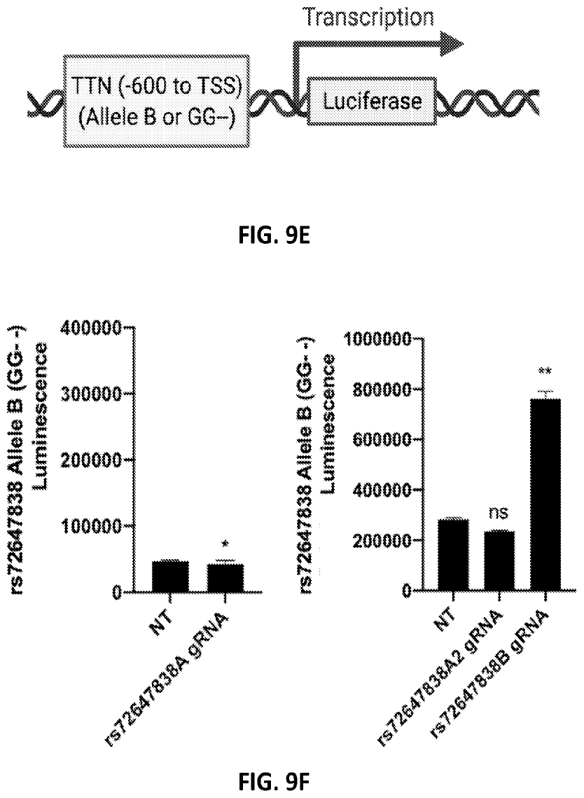

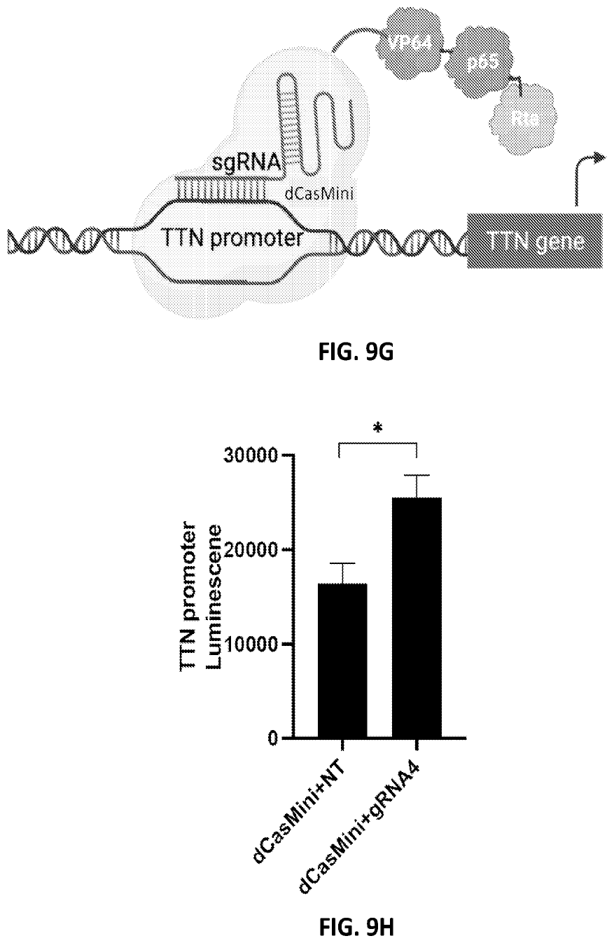

While TTN promoter activation using CRISPRa improved contractility deficits in TTNtvA^{+/-} DCM models in parallel with increasing full-length TTN protein levels (wildtype), it also resulted in increased truncated TTN protein that has been previously shown to sarcomere integrate and partially inhibit contractile function (Romano et al., Id.). An improved method for CRISPRa would be to increase full-length TTN, but not truncated TTN protein. To achieve this, common genetic variants were identified that localized to the N2BA TTN promoter or regulatory elements that could be adapted to provide allele specific CRISPRa (set forth in Table 7 of SEQ ID NOs: 42-95). Since individuals harboring these common genetic variants are commonly heterozygous for the polymorphism (set forth as an example in Table 8), allele specific CRISPRa could be developed by using gRNAs that perfectly align to either the major or minor polymorphic allele (illustrated in FIG. 9A). This is based on the premise that the common genetic variant would provide a distinct “landing pad” for dCas9-VPR, which would provide recognition sequences that are distinct between the two alleles (i.e., wildtype and TTNtv); see, FIG. 9C and FIG. 9F. For example, rs72647838 is localized within an ATAC-seq peak, is −300 base pairs upstream of the N2BA TIN TSS (shown in FIG. 9B) and differs by a two-nucleotide deletion that could be intentionally exploited for allele-specific CRISPRa. Previous CRISPRa studies have demonstrated that gRNAs more upstream of rs72647838 (e.g., NPX) relative to the TSS can activate TTN protein levels confirming that the SNP is sufficiently proximal to the TSS to achieve activation using dCas9-VPR. To provide allele specificity, gRNAs were designed with allele-specific PAM usage (e.g., gRNA rs72647838A), or spacer sequences (e.g., rs72647838A2 and rs72647838B) (shown in FIG. 9B). Two luciferase reporter constructs composed of the TTN promoter (−600 to 0 relative to the TTNN2BA TSS) harboring rs72647838 allele A (major allele or GGGG shown in FIG. 9C) or rs72647838 allele B (minor allele or GG—shown in FIG. 9E) were then generated. Using luciferase activity assays in CMs transduced with either allele A or allele B TTN promoters provided the capacity to determine whether allele-specific TTN activation was possible using a panel of gRNAs (set forth in Table 9 of SEQ ID NOs: 103-105). Indeed, it was found that rs72647838A and rs72647838A2 gRNAs (whose recognition sequences were a perfect match for allele A) activated only allele A TTN promoter (shown in FIG. 9D), while rs72647838B gRNA (whose recognition sequence was a perfect match for allele B) activated only allele B (shown in FIG. 9F). These results supported the conclusion that CRISPRa can be adapted to provide allele-specific activation through leveraging common genetic variation within TTN promoter and likely other regulatory elements.

Similar positive results were obtained when CRISPR enzymes other than SpCas9 were used (FIG. 9H). In these experiments, lentivirus encoding dCasMini-VPR (shown in FIG. 9G and sequences listed in Table 10 of SEQ ID NO: 106), a reengineered version of Cas12f that has been optimized for eukaryotic function (Xu et al., Id.), were used successfully, along with a gRNA recognizing the TTNN2BA promoter and a NT control gRNA (set forth in Table 11 of SEQ ID NO: 107 and SEQ ID NO: 108, and Table 12 of SEQ ID NO: 109) were co-transduced into CMs.

TABLE 7
Common genetic variants localized to the N2BA TTN promoter or regulatory elements.

| | | | | | | | 1000 | 1000 |
|---|---|---|---|---|---|---|---|---|
| Polymor- | | | Flanking sequence (5′ to | SEQ | Flanking sequence (5′ to | SEQ | -Genomes | -Genomes |
| phism | | | 3′; underline denotes | ID | 3′; underline denotes | ID | Allele A | Allele B |
| id | Location | Position (hg38) | Allele A) | NO | Allele B) | NO | proportion | proportion |
| rs2279472 | N2BA promoter | chr2: 178807687-178807687 | GACCATCTCCGACATGAATC | 42 | GACCATCTCTGACATGAATC | 69 | 0.765 | 0.235 |
| rs13422986 | N2BA promoter | chr2: 178807306-178807306 | gttaaaatccgaaaatgcag | 43 | gttaaaatctgaaaatgcag | 70 | 0.980 | 0.020 |
| rs72647839 | N2BA promoter | chr2: 178807735-178807735 | cttgtgggcagtcatacaaa | 44 | cttgtgggcggtcatacaaa | 71 | 0.951 | 0.049 |
| rs72647838 | N2BA promoter | chr2: 178807750-178807753 | caaaagagccccaaacctga | 45 | caaaagagccaaacctga | 72 | 0.890 | 0.110 |
| rs2046778 | N2BA promoter | chr2: 178807820-178807820 | caaaccaaatggacctgtc | 46 | caaaccaaacggacctgtc | 73 | 0.799 | 0.201 |
| rs10179219 | N2BA promoter | chr2: 178807850-178807850 | cagatgaggaaatttatctc | 47 | cagatgagggaatttatctc | 74 | 0.902 | 0.098 |
| rs12465459 | N2BA promoter | chr2: 178807984-178807984 | tacactgatgttaatgagct | 48 | tacactgatgctaatgagct | 75 | 0.678 | 0.322 |
| rs11693372 | N2BA promoter | chr2: 178808637-178808637 | acaagatgacgccatgtact | 49 | acaagatgatgccatgtact | 76 | 0.679 | 0.321 |
| rs10930835 | N2BA promoter | chr2: 178809919-178809919 | atatttattaaacatatagt | 50 | atatttattgaacatatagt | 77 | 0.669 | 0.331 |
| rs10190488 | TTN Element 1 | chr2: 178819671-178819671 | ccataaaggtgactttttaa | 51 | ccataaaggggactttttaa | 78 | 0.621 | 0.379 |
| rs10189747 | TTN Element 1 | chr2: 178818913-178818913 | aagcatttgtaaaggcgact | 52 | aagcatttgcaaaggcgact | 79 | 0.639 | 0.361 |
| rs998329 | TTN Element 1 | chr2: 178819512-178819512 | ttattttctgaattgtactg | 53 | ttattttctaaattgtactg | 80 | 0.668 | 0.332 |
| rs10178003 | TTN Element 1 | chr2: 178819739-178819739 | ccccccaaacacacagaatt | 54 | ccccccaaaaacacagaatt | 81 | 0.532 | 0.468 |
| rs10497526 | TTN Element 1 | chr2: 178820112-178820112 | tccataacacggcagttgat | 55 | tccataacatggcagttgat | 82 | 0.870 | 0.130 |
| rs1489486 | TTN Element 1 | chr2: 178820256-178820256 | gtgagctacattcctgcttc | 56 | gtgagctacgttcctgcttc | 83 | 0.149 | 0.851 |
| rs1489484 | TTN Element 1 | chr2: 178820417-178820417 | taattttacaaatcggattc | 57 | taattttactaatcggattc | 84 | 0.613 | 0.387 |
| rs3045726 | TTN Element 1 | chr2: 178821103-178821103 | catcaactagctatattccc | 58 | catcaactagtatgtgctatattccc | 85 | 0.588 | 0.412 |
| rs2046777 | TTN Element 1 | chr2: 178821210-178821210 | gtttgggtataccaagtttt | 59 | gtttgggtacaccaagtttt | 86 | 0.589 | 0.411 |
| rs7586208 | TTN Element 1 | chr2: 178821768-178821768 | acaggtgctggagaggatgt | 60 | acaggtgctagagaggatgt | 87 | 0.674 | 0.326 |
| rs3045731 | TTN Element 3 | chr2: 178837851-178837852 | aacctacaaggttaaaat | 61 | aacctacaaggggttaaaat | 88 | 0.114 | 0.886 |
| rs2078403 | TTN Element 3 | chr2: 178837883-178837883 | caagaattacggatgagaaa | 62 | caagaattatggatgagaaa | 89 | 0.335 | 0.665 |
| rs1489481 | TTN Element 3 | chr2: 178838675-178838675 | gtggaggagccagatattaa | 63 | gtggaggaggcagatattaa | 90 | 0.190 | 0.810 |
| rs75260489 | TTN Element 3 | chr2: 178839608-178839608 | aaaaaaaaatgtgttttcag | 64 | aaaaaaaaaagtgttttcag | 91 | 0.454 | 0.546 |
| rs13033688 | TTN Element 4 | chr2: 178853406-178853406 | tttgttcaatggccaatcac | 65 | tttgttcaaaggccaatcac | 92 | 0.892 | 0.108 |
| rs12990752 | TTN Element 4 | chr2: 178854353-178854353 | gaaaccccatctctactaaa | 66 | gaaaccccacctctactaaa | 93 | 0.724 | 0.276 |
| rs1872203 | TTN Element 4 | chr2: 178855239-178855239 | aggagcatgatacatgaata | 67 | aggagcatgttacatgaata | 94 | 0.619 | 0.381 |
| rs10803920 | TTN Element 5 | chr2: 178863436-178863436 | ctcaaaaattcattgcaagt | 68 | ctcaaaaatgcattgcaagt | 95 | 0.114 | 0.886 |

TABLE 8

| | Allele | Allele | Allele |
|---|---|---|---|
| Population | Count | Number | Frequency |
| Ashkenazi Jewish | 1120 | 3458 | 0.3239 |
| Middle Eastern | 90 | 316 | 0.2848 |
| European (non-Finnish) | 15776 | 67952 | 0.2322 |
| Other | 479 | 2086 | 0.2296 |
| Latino/Admixed American | 2646 | 15248 | 0.1735 |
| African/African American | 5545 | 41338 | 0.1341 |
| European (Finnish) | 1298 | 10568 | 0.1228 |
| Amish | 104 | 912 | 0.1140 |
| South Asian | 360 | 4806 | 0.07491 |
| East Asian | 4 | 5182 | 0.0007719 |
| XX | 14592 | 77708 | 0.1878 |
| XY | 12830 | 74158 | 0.1730 |
| Total | 27422 | 151866 | 0.1806 |

TABLE 9
List of gRNAs targeted to TTN promoter sites harboring rs72647838 allele A or
rs72647838 allele B in FIG. 9D and FIG. 9F.

| id | Recognition sequence | PAM | hg38 gRNA location | SEQ ID NO |
|---|---|---|---|---|
| rs72647838A | GGGCACAGTCCTCAGGTTTG | GGG | chr2: 178,807, 753-178, 807, | 103 |
| | | | 772 | |
| rs72647838A2 | ACAAAAGAGCCCCAAACCTG | AGG | chr2: 178,807, 741-178, 807, | 104 |
| | | | 760 | |
| rs72647838B | ATACAAAAGAGCCAAACCTG | AGG | chr2: 178,807, 739-178, 807, | 105 |
| | | | 760 | |

TABLE 10
dCasMini-VPR sequence

atgggacccaagaaaaaacgcaaggtgggaagcggatccgccaaaaacaccattaccaaaacactgaaactgcgtattgtgcgtccg
tataatagcgcagaagtggaaaaaattgttgccgacgaaaaaaacaaccgcgaaaaaatcgcactggaaaagaacaaagacaaagtg
aaagaagcctgcagcaaacatctgaaagttgcagcatattgtaccacacaggttgaacgtaatgcatgcctgttttgtaaagcacgtaaac
tggatgacaaattctaccaaaaactgcgtggtcagtttccggatgcagttttttggcaagaaatcagcgaaatttttcgccagctgcagaaa
caggcagcagaaatctataatcagagcctgatcgaactgtactacgagatttttatcaaaggcaaaggtattgcaaatgccagcagcgtt
gaacattatctgagtagagtttgttatagacgtgcagcagaactgtttaaaaacgcagcaattgcaagcggtctgcgtagcaaaatcaaaa
gcaattttcgtctgaaagaactgaaaaacatgaaaagtggtctgccgaccaccaaaagcgataattttccgattccgctggttaaacagaa
aggtggtcagtataccggttttgaaattagcaatcataatagcgacttcatcatcaagattccgtttggtcgttggcaggtcaaaaaagagat
tgataaatatcgtccgtgggagaaatttgactttgaacaggttcagaaaagcccgaaaccgattagcctgctgctgagcacccagcgtcg
taaacgtaataaaggttggagcaaagatgaaggcaccgaagccgaaatcaaaaaagttatgaatggcgattatcagaccagctacattg
aagttaaacgtggcagcaaaatctgtgaaaaaagcgcatggatgctgaatctgagcattgatgttccgaaaattgataaaggtgtggatc
cgagcattattggtggtattgcagttggtgttagatcaccgctggtttgcgcaattaacaatgcatttagccgttatagcatcagcgataacg
acctgtttcacttcaacaagaaaatgtttgcacgtcgtcgtatcctgctgaaaaaaaaccgtcataaacgtgcaggtcatggtgcaaaaaa
caaactgaaaccgatcaccattctgaccgaaaaaagtgaacgttttcgcaaaaagctgattgaacgttgggcatgtgaaatcgcggatttc
ttcattaaaaacaaagttggcaccgtgcagatggaaaatctggaaagcatgaaacgtaaagaggacagctattttaacattcgcctgcgt
ggcttttggccgtatgcagaaatgcagaacaaaatcgaattcaaactgaagcagtatggcatcgaaattcgtaaagttgcaccgaataata
ccagcaaaacctgtagcaaatgtggccatctgaacaactatttcaacttcgagtaccgcaagaaaaacaaattcccgcactttaaatgcga
aaaatgcaacttcaaagaaaacgccgcgtataatgcagccctgaatatttcaaacccgaaactgaaaagcaccaaagagagaccggcc
tatccctatgacgtgcccgattatgccagcctgggcagcggcgacggcattggtagtgggagcaacggcagcagcctcgatgctttag
acgattttgacttagatatgcttggttcagacgcgttagacgacttcgacctagacatgttaggctcagatgcattggacgacttcgatttag
atatgttgggctccgatgccctagatgactttgatctagatatgctaggtagtggcggcagcggatcccagtatctgcccgacacagatga
tagacaccgaatcgaagagaaacgcaagcgaacgtatgaaaccttcaaatcgatcatgaagaaatcgcccttctcgggtccgaccgat
cccaggcccccaccgagaaggattgcggtcccgtcccgctcgtcggccagcgtgccgaagcctgcgccgcagccctaccccttcac
gtcgagcctgagcacaatcaattatgacgagttcccgacgatggtgttcccctcgggacaaatctcacaagcctcggcgctcgcaccag
cgcctccccaagtccttccgcaagcgcctgccccagcgcctgcaccggcaatggtgtccgccctcgcacaggcccctgcgcccgtcc
ccgtgctcgcgcctggaccgccccaggcggtcgctccaccggctccgaagccgacgcaggccggagagggaacactctccgaagc
acttcttcaactccagtttgatgacgaggatcttggagcactccttggaaactcgacagaccctgcggtgtttaccgacctcgcgtcagta
gataactccgaatttcagcagcttttgaaccagggtatcccggtcgcgccacatacaacggagcccatgttgatggaataccccgaagc
aatcacgagacttgtgacgggagcgcagcggcctcccgatcccgcacccgcacctttgggggcacctggcctccctaacggacttttg
agcggcgacgaggatttctcctccatcgccgatatggatttctcagccttgctgtcacagatttccagcggctctggcagcggcagccgg
gattccagggaagggatgtttttgccgaagcctgaggccggctccgctattagtgacgtgtttgagggccgcgaggtgtgccagccaaa
acgaatccggccatttcatcctccaggaagtccatgggccaaccgcccactccccgccagcctcgcaccaacaccaaccggtccagta
catgagccagtcgggtcactgaccccggcaccagtccctcagccactggatccagcgcccgcagtgactcccgaggccagtcacctg
ttggaggatcccgatgaagagacgagccaggctgtcaaagcccttcgggagatggccgatactgtgattccccagaaggaagaggct
gcaatctgtggccaaatggacctttcccatccgcccccaaggggccatctggatgagctgacaaccacacttgagtccatgaccgagg
atctgaacctggactcacccctgaccccggaattgaacgagattctggataccttcctgaacgacgagtgcctcttgcatgccatgcatat
cagcacaggactgtccatcttcgacacatctctgtttcatatgggtggaggctccggggaagatcctgctgccaaacgcgttaaactaga
ctga (SEQ ID NO: 106)

TABLE 11

| id | Recognition sequence | PAM | hg38 gRNA location | SEQ ID NO |
|---|---|---|---|---|
| NT | cgaatacgcccacgcgatgggta | n/a | n/a | 107 |
| gRNA4 | gcactgtcaatcagaagaacagg | TTTA | chr2: | 108 |
| | | | 178, 807, 559-178, 807, 581 | |

TABLE 12
U6-dCasMini-VPR gRNA (23 mer recognition sequence N23)

GAGGGCCTATTTCCCATGATTCCTTCATATTTGCATATACGATACAAGGCTGTTAG
AGAGATAATTAGAATTAATTTGACTGTAAACACAAAGATATTAGTACAAAATAC
GTGACGTAGAAAGTAATAATTTCTTGGGTAGTTTGCAGTTTTAAAATTATGTTTTA
AAATGGACTATCATATGCTTACCGTAACTTGAAAGTATTTCGATTTCTTGGCTTTA
TATATCTTGTGGAAAGGACGAAACACCGGGCTTCACTGATAAAGTGGAGAACCG
CTTCACCAAAAGCTGTCCCTTAGGGGATTAGAACTTGAGTGAAGGTGGGCTGCTT
GCATCAGCCTAATGTCGAGAAGTGCTTTCTTCGGAAAGTAACCCTCGAAACAAAT
TCATTTGAATGAAGGAATGCAACnnnnnnnnnnnnnnnnnnnnnnnTTTTTTTAAGCTTGG
GCCGCTCGAGGGATCCATTAGGCGGCCGCG (SEQ ID NO: 109)

### Example 7: CRISPRa Restored Sarcomere Content and Contractility Deficits

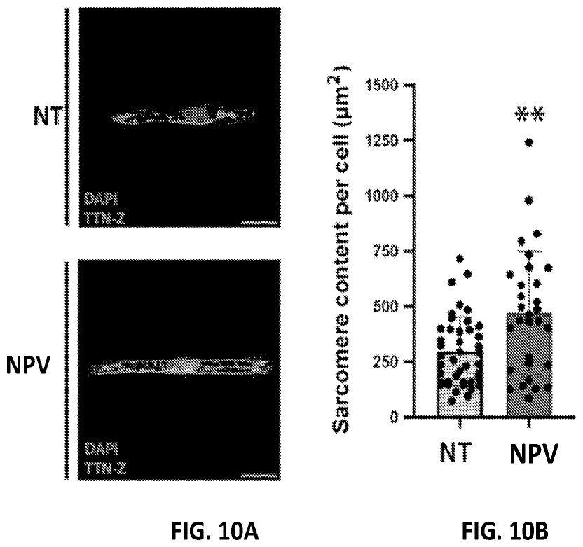

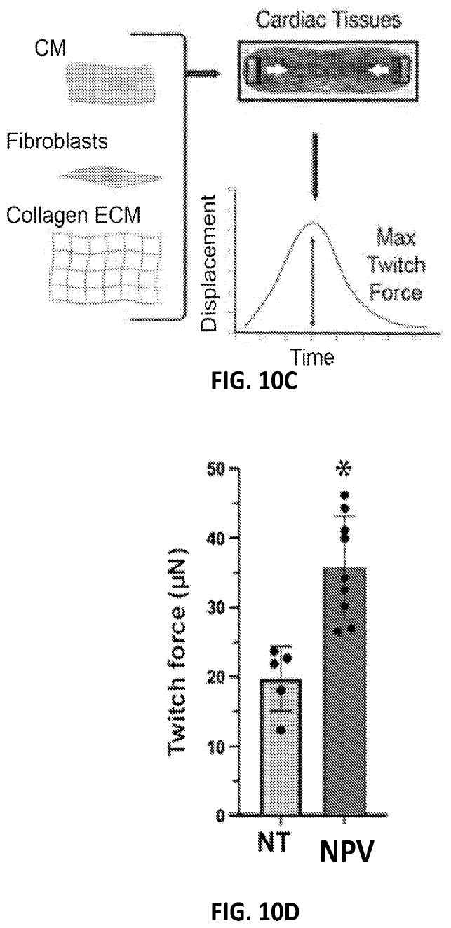

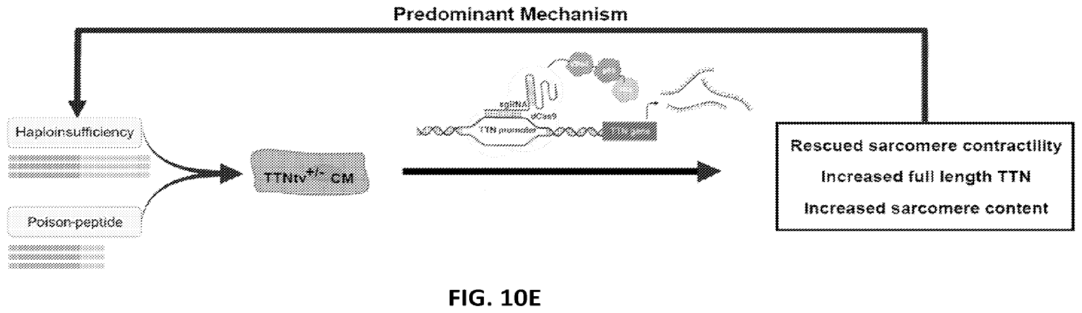

As reductions in sarcomere content and contractility are functional consequences of TTNtvs in CMs (Hinson et al., 2015, Science 349:982-6; Romano et al., 2022, Circulation 145:194-205; Chopra et al., 2018, Dev. Cell. 44:87-96 e5), how TTN CRISPRa influenced these functional parameters was determined. To determine sarcomere content, dCas9-VPR-TTNtv+/− CMs with NPV or NT gRNAs were transduced, and replated CMs onto 2000 μm fibronectin rectangles (7:1 aspect ratio) as described previously to optimize sarcomere organization and maturity (Clippinger et al., 2019, Proc Natl Acad Sci USA 116:17831-1784037; Ribeiro et al., 2015, Proc Natl Acad Sci USA 112:12705-10). Micropatterned CMs were next fixed and immunostained with an anti-sarcomere antibody (anti-TTN). CM sarcomere area was quantified using confocal microscopy and a custom ImageJ script (FIG. 10A). TTN CRISPRa using NPV gRNA increased average CM sarcomere area relative to NT gRNA controls (FIG. 10B). Finally, contractility was quantified after TTN CRISPRa using custom biomimetic 3-dimensional CMTs (FIG. 10C) as described previously (Cohn et al., 2019, Stem Cell Reports 12: 71-8311; Romano et al., 2022, Circulation 145: 194-205). TTN CRISPRa using NPV gRNA increased CMT twitch force relative to NT gRNA controls (FIG. 10D). Taken together, TTN CRISPRa rescued sarcomere content and CMT contractility deficits secondary to a DCM-associated TTNtv (FIG. 10E) were determined. Furthermore, these functional improvements support the model that haploinsufficiency is the predominant genetic mechanism underlying TTNtvs (and that the single wildtype TTN allele produces insufficient normal TTN protein levels, while the dominant negative hypothesis implicates a deleterious gain-of-function such as by toxic truncated TTN protein aggregation or through disturbance of normal TTN function by competitive sarcomere integration of truncated TTN poison peptides) and TTN CRISPRa and likely other methods to augment TTN protein levels could be a therapeutic for other DCM-associated TTN variants.

### Example 8: In Vivo Ttn Activation as a Therapeutic for Dilated Cardiomyopathy and Other Heart Failure Types

To validate TIN activation studies from human cardiac microtissue models, a custom knock-in mouse model was used, of dilated cardiomyopathy secondary to heterozygous Ttn truncation variants (Ttntvs+/−), and a humanized TTN model that has the mouse Tin promoter replaced with the human TTN promoter. The human TTN promoter model enables testing of TTN activation treatments that can be directly applied to humans that have been previously validated in human cardiac microtissues. Like human hearts (McAfee et al., 2021, Sci Transl Med. 13:eabd7287) and cardiac microtissue models (Romano et al., 2021, Circulation 145:194-205; Hinson et al., 2015, Science 349:982-6) Ttntv+/− knock-in mouse models both recapitulate the molecular consequences of TTNtvs (i.e., express truncated Ttn and reduced full-length Ttn proteins), the functional changes by echocardiography (i.e., reduced cardiac ejection fraction and increased chamber size), and the histopathological changes by staining (i.e., increased percentage of cardiac fibrosis) (Gramlich et al., 2009, J Mol Cell Cardiol. 47:352-8). In addition, cardiac stressors (e.g., osmotic pump delivery of chronic isoproterenol that activates beta-adrenergic signaling or chronic angiotensin II that activates the AT1 receptor) exacerbate the functional consequences of Ttntvs in mice (Gramlich et al., 2009, J Mol Cell Cardiol. 47:352-8) after >1 week of treatment, thus providing an efficient platform for therapeutic screening.

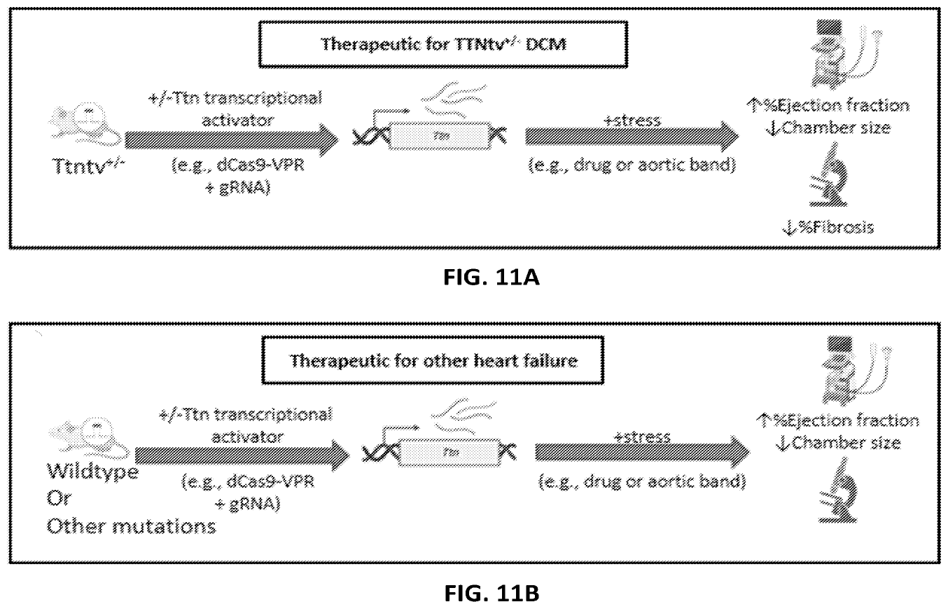

In this validation study, dCas9-VPR was tested as an example transcriptional activator to parallel cardiac microtissue studies, and guide RNAs that are programmed to recognize either the mouse Ttn promoter, the human TTN promoter or DNA regulatory elements controlling promoter activity such as Ttn enhancer sequences. In addition to dCas9-VPR, other transcriptional activators can be tested such as other CRISPR/Cas proteins, or other programmable chimeric activator systems (e.g., any programmable DNA binding protein that is conjugated to a transcriptional activator) such as but not limited to TALE nucleases or Zinc fingers conjugated to VP64 or other transcriptional activator enzymes. TTN activators are delivered through either transgenic approaches or with viral vectors including but not limited to adeno-associated vectors such as AAV9 and its derivatives that are strongly cardiotropic particularly in combination with cardiac-specific promoters such as from troponin T (Prasad et al., 2011, Gene Ther. 18:43-52). Given sufficient time for Ttn activation such as but not limited to 2 weeks post-injection of AAVs delivering dCas9-VPR and programmed guide RNA, Ttntv+/− mice are treated with cardiac stressors to induce dilated cardiomyopathy phenotypes (FIG. 11A). Functional assessments to evaluate efficacy and safety of TTN activation include echocardiography for cardiac structure and function, histopathology for quantification of cardiac fibrosis, and electrophysiology for arrhythmia risk. Therapeutic efficacy is accomplished when TTN activation results in increased cardiac ejection fraction, and/or reduced cardiac chamber enlargement, and/or reduced cardiac fibrosis compared to affected individuals. Based on a DCM c.43628insAT truncation mutation (a TTNJ2-bp insertion mutation) knock-in mouse model, it is expected that Ttntv+/− mice will exhibit about 23% stress-induced reduction in cardiac ejection fraction, and about 13% increase in cardiac chamber size. Therapeutic intervention with TTN activation in Ttntv+/− mice can potentially cause 15% improvement in ejection fraction and 10% decline in chamber size. TTN activation is tested also in other forms of heart failure such as from other genetic mutations such as in other gene mutations that cause human DCM (e.g., RBM20, LMNA, MYH7, TNNT2, TNNI3, TNNC1, TPM1, BAG3, and others as described (Hershberger et al., 2021, Circ Res. 128:1514-1532)), as well as but not limited to other environmental models of heart failure including transverse aortic banding (TAC) to generate pressure overload, or chemical stressors including but not limited to chronic treatments with isoproterenol or angiotensin II (FIG. 11B).

Those skilled in the art will recognize or be able to ascertain using no more than routine experimentation, many equivalents to the specific embodiments of the invention described herein. Such equivalents are intended to be encompassed by the following claims.

We claim:

1. A method for ameliorating dilated cardiomyopathy (DCM) in a subject in need thereof, comprising:
delivering to target tissue in the subject a therapeutically effective amount of a composition capable of introducing a transcriptional activator at a site specific for a regulatory sequence controlling or affecting TTN gene expression,
wherein expression of a functional TTN gene product is increased in the subject's heart tissue or skeletal muscle tissue.

2. The method of claim 1, wherein DCM in the subject is caused by one TTN allele in the subject's genomic DNA encoding a TTN gene that produces a dysfunctional Titin protein gene product.

3. The method of claim 2, wherein the TTN allele encodes a truncated mutation of the TTN gene.

4. The method of claim 2, wherein the TTN allele encodes a nonsense mutation of the TTN gene.

5. The method of claim 2, wherein the TTN allele encodes a frameshift mutation of the TTN gene.

6. The method of claim 2, wherein the TTN allele encodes a splicing variant mutation of the TTN gene.

7. The method of claim 2, wherein the TTN allele encodes for a variant that reduces TTN expression levels.

8. The method of claim 1, wherein the target tissue is heart tissue.

9. The method of claim 1, wherein the target tissue is skeletal muscle tissue.

10. The method of claim 1, wherein the composition delivered to target tissue is a CRISPR-Cas9 complex, comprising a nuclease-dead Cas9 protein linked to an activator protein and a guide RNA specific for a regulatory sequence controlling or affecting TTN gene expression.

11. The method of claim 10, wherein the nuclease-dead Cas9 protein linked to an activator protein is dCas9-VPR.

12. The method of claim 10, wherein the nuclease-dead Cas9 protein linked to an activator protein is dCas9-VP64.

13. The method of claim 10, wherein the nuclease-dead Cas9 protein linked to an activator protein is dCas9-SunTag.

14. The method of claim 10, wherein the nuclease-dead Cas9 protein linked to an activator protein is dCas9-SAM.

15. The method of claims 1-14, wherein the regulatory sequence for TTN gene expression is located within TTN gene promoter region.

16. The method of claim 15, wherein the guide RNA targeting the TTN gene promoter region is a sgRNA having a sequence identified by any one of SEQ ID NO: 9, SEQ ID NOs: 13-21, SEQ ID NOs: 23-25, SEQ ID NOs: 27-32, and SEQ ID NOs: 38-41.

17. The method of claim 15, wherein the guide RNA targeting the TTN gene promoter region is a sgRNA having a sequence identified by any one of SEQ ID NOs: 13, 14, 15, 21, 24, 25, 27, 28, 30, 31, and 38-41.

18. The method of claims 1-14, wherein the regulatory sequence for TTN gene expression is located within TTN gene enhancer region.

19. The method of claim 18, wherein the guide RNA targeting the TTN gene enhancer region is a sgRNA having a sequence identified by any one of SEQ ID NO: 33, and SEQ ID NOs: 35 through 37.

20. The method of claim 18, wherein the guide RNA is a sgRNA having a sequence identified by SEQ ID NO: 33.

21. The method of claims 10-14 wherein the CRISPR-Cas9 complex delivered to target tissue in the subject is delivered by one or a plurality of expression constructs encoding the nuclease-dead Cas9 protein linked to an activator protein and a guide RNA specific for regulatory sequences for TTN gene expression.

22. The method of claim 21 wherein the CRISPR-Cas9 complex or one or a plurality of expression constructs are formulated for delivery to heart tissue or skeletal muscle tissue.

23. A sgRNA molecule identified by any of SEQ ID NO: 9, SEQ ID NOs: 13-21, SEQ ID NOs: 23-25, SEQ ID NOs: 27-32, SEQ ID NO: 33, SEQ ID NOs: 35-37, and SEQ ID NOs: 38-41.

24. A pharmaceutical composition comprising a CRISPR-Cas9 complex, wherein the CRISPR-Cas9 complex comprising a nuclease-dead Cas9 protein linked to an activator protein; and a guide RNA specific for a regulatory sequence controlling or affecting TTN gene expression, and a pharmaceutically acceptable carrier.

25. The pharmaceutical composition of claim 24, wherein the nuclease-dead Cas9 protein linked to an activator protein is dCas9-VPR.

26. The pharmaceutical composition of claim 24, wherein the nuclease-dead Cas9 protein linked to an activator protein is dCas9-VP64.

27. The pharmaceutical composition of claim 24, wherein the nuclease-dead Cas9 protein linked to an activator protein is dCas9-SunTag.

28. The pharmaceutical composition of claim 24, wherein the nuclease-dead Cas9 protein linked to an activator protein is dCas9-SAM.

29. The pharmaceutical composition of claims 24-28, wherein the regulatory sequence for TTN gene expression is located within TTN gene promoter region.

30. The pharmaceutical composition of claim 29, wherein the guide RNA targeting the TTN gene promoter region is a sgRNA having a sequence identified by any one of SEQ ID NO: 9, SEQ ID NOs: 13-21, SEQ ID NOs: 23-25, SEQ ID NOs: 27-32, and SEQ ID NOs: 38-41.

31. The pharmaceutical composition of claim 29, wherein the guide RNA targeting the TTN gene promoter region is a sgRNA having a sequence identified by any one of SEQ ID NOs: 13, 14, 15, 21, 24, 25, 27, 28, 30, 31, and 38-41.

32. The pharmaceutical composition of claims 24-28, wherein the regulatory sequence for TTN gene expression is located within TTN gene enhancer region.

33. The pharmaceutical composition of claim 32, wherein the guide RNA targeting the TTN gene enhancer region is a sgRNA having a sequence identified by any one of SEQ ID NO: 33, and SEQ ID NOs: 35-37.

34. The pharmaceutical composition of claim 32, wherein the guide RNA is a sgRNA having a sequence identified by SEQ ID NO: 33.

35. The pharmaceutical composition of claims 24-28, wherein the CRISPR-Cas9 complex delivered to target tissue in the subject is delivered by one or a plurality of expression constructs encoding the nuclease-dead Cas9 protein linked to an activator protein and a guide RNA specific for regulatory sequences for TTN gene expression.

36. The pharmaceutical composition of claim 35, wherein the CRISPR-Cas9 complex or one or a plurality of expression constructs are formulated for delivery to heart tissue or skeletal muscle tissue.

37. The pharmaceutical composition of claim 24, comprising the guide RNA molecule identified by any of SEQ ID NO: 9, SEQ ID NOs: 13-21, SEQ ID NOs: 23-25, SEQ ID NOs: 27-32, SEQ ID NO: 33, SEQ ID NOs: 35-37, and SEQ ID NOs: 38-41.

38. A method for ameliorating dilated cardiomyopathy (DCM) in heart tissue of a subject in need thereof, comprising:
delivering to target tissue in the subject a therapeutically effective amount of a composition capable of introducing a transcriptional activator at a site specific for a regulatory sequence controlling or affecting a wild type TTN allele that produces a functional Titin protein gene product,
wherein expression of the functional Titin protein is specifically increased in the subject's heart tissue or skeletal muscle tissue.

39. The method of claim 38, wherein DCM is caused by a mutated TTN allele in the subject's genomic DNA encoding a TTN gene that produces a dysfunctional Titin protein gene product.

40. The method of claim 39, wherein the regulatory sequence of the wild type TTN allele contains a polymorphic variant, and wherein the polymorphic variant is not present in the regulatory sequence of the mutated TTN allele.

41. The method of claim 39, wherein the mutated TTN allele encodes a truncated mutation of the TTN gene.

42. The method of claim 39, wherein the mutated TTN allele encodes a nonsense mutation of the TTN gene.

43. The method of claim 39, wherein the mutated TTN allele encodes a frameshift mutation of the TTN gene.

44. The method of claim 39, wherein the mutated TTN allele encodes a splicing variant mutation of the TTN gene.

45. The method of claim 39, wherein the mutated TTN allele encodes for a variant that reduces TTN expression levels.

46. The method of claim 38, wherein the target tissue is heart tissue.

47. The method of claim 38, wherein the target tissue is skeletal muscle tissue.

48. The method of claim 38, wherein the composition delivered to target tissue is a CRISPR-Cas9 complex, comprising a nuclease-dead Cas9 protein linked to an activator protein and a guide RNA specific for a regulatory sequence controlling or affecting the wild type TTN allele that produces the functional Titin protein gene product.

49. The method of claim 48, wherein the nuclease-dead Cas9 protein linked to an activator protein is dCas9-VPR.

50. The method of claim 48, wherein the nuclease-dead Cas9 protein linked to an activator protein is dCas9-VP64.

51. The method of claim 48, wherein the nuclease-dead Cas9 protein linked to an activator protein is dCas9-SunTag.

52. The method of claim 48, wherein the nuclease-dead Cas9 protein linked to an activator protein is dCas9-SAM.

53. The method of claims 38-52, wherein the regulatory sequence for TTN gene expression is located within the wild type TTN allele gene promoter region.

54. The method of claims 38-52, wherein the regulatory sequence for TTN gene expression is located within the wild type TTN allele gene enhancer region.

55. The method of claim 53 or 54, wherein the guide RNA is a sgRNA targeting the wild type TTN allele gene promoter region or the wild type TTN allele gene enhancer region having a sequence identified by any one of SEQ ID NOs: 103-105.

56. The method of claims 48-52, wherein the CRISPR-Cas9 complex delivered to target tissue in the subject is delivered by one or a plurality of expression constructs encoding the nuclease-dead Cas9 protein linked to an activator protein and a guide RNA specific for regulatory sequences for the wild type TTN allele that produces the functional Titin protein gene product.

57. The method of claim 56, wherein the CRISPR-Cas9 complex or one or a plurality of expression constructs are formulated for delivery to heart tissue or skeletal muscle tissue.

58. A sgRNA molecule identified by any of SEQ ID NOs: 103-105.

59. A pharmaceutical composition comprising a CRISPR-Cas9 complex, wherein the CRISPR-Cas9 complex comprising a nuclease-dead Cas9 protein linked to an activator protein; and a sgRNA specific for a regulatory sequence controlling or affecting a wild type TTN allele that produces a functional Titin protein gene product.

60. The method of claim 59, wherein the nuclease-dead Cas9 protein linked to an activator protein is dCas9-VPR.

61. The method of claim 59, wherein the nuclease-dead Cas9 protein linked to an activator protein is dCas9-VP64.

62. The method of claim 59, wherein the nuclease-dead Cas9 protein linked to an activator protein is dCas9-SunTag.

63. The method of claim 59, wherein the nuclease-dead Cas9 protein linked to an activator protein is dCas9-SAM.

64. The method of claims 59-63, wherein the regulatory sequence for TTN gene expression is located within the wild type TTN allele gene promoter region.

65. The method of claims 59-63, wherein the regulatory sequence for TTN gene expression is located within the wild type TTN allele gene enhancer region.

66. The method of claim 64 or 65, wherein the guide RNA targeting the wild type TTN allele gene promoter region or the wild type TTN allele gene enhancer region is a sgRNA having a sequence identified by any one of SEQ ID NOs: 103-105.

67. The method of claims 59-63, wherein the CRISPR-Cas9 complex delivered to target tissue in the subject is delivered by one or a plurality of expression constructs encoding the nuclease-dead Cas9 protein linked to an activator protein and a guide RNA specific for regulatory sequences for the wild type TTN allele that produces the functional Titin protein gene product.

68. The method of claim 67, wherein the CRISPR-Cas9 complex or one or a plurality of expression constructs are formulated for delivery to heart tissue or skeletal muscle tissue.

69. The pharmaceutical composition of claim 59, comprising the guide RNA molecule identified by any of SEQ ID NOs. 103 through 105.

70. A CRISPR-Cas9 complex comprising a nuclease-dead Cas9 protein linked to an activator protein and a guide RNA specific for regulatory sequences for TTN gene expression.

71. The method of claim 70, wherein the CRISPR-Cas9 complex is formulated for delivery to heart tissue.

72. The method of claim 70, wherein the CRISPR-Cas9 complex is formulated for delivery to skeletal muscle tissue.

73. A CRISPR-Cas9 complex comprising a nuclease-dead Cas9 protein linked to an activator protein and a guide RNA specific for regulatory sequences for a wild type TTN allele that produces a functional Titin protein gene product.

74. The method of claim 73, wherein the CRISPR-Cas9 complex is formulated for delivery to heart tissue.

75. The method of claim 73, wherein the CRISPR-Cas9 complex is formulated for delivery to skeletal muscle tissue.
